SEQUENCE LISTING (24 sequences)

SEQ ID NO: 1 - RNA, 20 nt, synthetic construct
  source 1..20: mol_type = other RNA
atgtagactgccaaagtgta

SEQ ID NO: 2 - RNA, 20 nt, synthetic construct
  source 1..20: mol_type = other RNA
tgtagactgccaaagtgtat

SEQ ID NO: 3 - RNA, 20 nt, synthetic construct
  source 1..20: mol_type = other RNA
agtgtatgggatgctttcaa

SEQ ID NO: 4 - RNA, 20 nt, synthetic construct
  source 1..20: mol_type = other RNA
tcttcttcagtaatgttgca

SEQ ID NO: 5 - DNA, 20 bp, synthetic construct
  source 1..20: mol_type = other DNA
ctatcagccactaatgaagt

SEQ ID NO: 6 - DNA, 20 bp, synthetic construct
  source 1..20: mol_type = other DNA
tatcagccactaatgaagtt

SEQ ID NO: 7 - DNA, 20 bp, synthetic construct
  source 1..20: mol_type = other DNA
aattaccttgttgcaaggta

SEQ ID NO: 8 - DNA, 20 bp, synthetic construct
  source 1..20: mol_type = other DNA
ctccactgctggcgccacct

SEQ ID NO: 9 - DNA, 20 bp, synthetic construct
  source 1..20: mol_type = other DNA
gcgccagcagtggagcggtc

SEQ ID NO: 10 - DNA, 20 bp, synthetic construct
  source 1..20: mol_type = other DNA
cgccagcagtggagcggtcc

SEQ ID NO: 11 - DNA, 20 bp, synthetic construct
  source 1..20: mol_type = other DNA
tttcctgcaagaatatctac

SEQ ID NO: 12 - DNA, 20 bp, synthetic construct
  source 1..20: mol_type = other DNA
ggggccactagggacaggat

SEQ ID NO: 13 - RNA, 20 nt, synthetic construct
  source 1..20: mol_type = other RNA
ctccaggttctcatcaatgc

SEQ ID NO: 14 - DNA, 20 bp, synthetic construct
  source 1..20: mol_type = other DNA
gtcaccaatcctgtccctag

SEQ ID NO: 15 - DNA, 20 bp, synthetic construct
  source 1..20: mol_type = other DNA
acagcatgtttgctgcctcc

SEQ ID NO: 16 - DNA, 20 bp, synthetic construct
  source 1..20: mol_type = other DNA
tgagttcccaacttcattag

SEQ ID NO: 17 - DNA, 20 bp, synthetic construct
  source 1..20: mol_type = other DNA
tcagaccgtaccttgcaaca

SEQ ID NO: 18 - DNA, 20 bp, synthetic construct
  source 1..20: mol_type = other DNA
gtaccttgcaacaaggtaat

SEQ ID NO: 19 - DNA, 20 bp, synthetic construct
  source 1..20: mol_type = other DNA
taccttgcaacaaggtaatt

SEQ ID NO: 20 - DNA, 20 bp, synthetic construct
  source 1..20: mol_type = other DNA
ttggccatagggctccaggc

SEQ ID NO: 21 - DNA, 903 bp, unidentified
  source 1..903: mol_type = unassigned DNA
atggccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgagacatgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctga

SEQ ID NO: 22 - protein, 300 aa, unidentified
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 23 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFFCKNIYRPDKFLQCVKNPEDSSCTSEI

SEQ ID NO: 24 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQAVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

(19) United States
(12) Patent Application Publication
(10) Pub. No.: US 2025/0262300 A1
(43) Pub. Date: Aug. 21, 2025

(54) SYSTEMS, METHODS, AND KITS FOR GENERATING AND ADMINISTERING ENGINEERED T CELLS AND CD38 TARGETING COMPOUNDS AS A COMBINATION THERAPY

(71) Applicant: Kite Pharma, Inc., Santa Monica, CA (US)

(72) Inventors: Katherine A. Freitas, Redwood City, CA (US); Geetha Boddu, Berkeley, CA (US); Chloe Knights Krishnamurty, Berkeley, CA (US); Sophie H. Viaud, San Francisco, CA (US)

(21) Appl. No.: 19/052,681

(22) Filed: Feb. 13, 2025

Related U.S. Application Data
(60) Provisional application No. 63/554,420, filed on Feb. 16, 2024.

(51) Int. Cl. A61K 40/11; A61K 40/31; C07K 16/28; C12N 5/0783

(52) U.S. Cl. CPC A61K 40/11; A61K 40/31; C07K 16/2896; C12N 5/0636; C12N 2510/00

(57) ABSTRACT

The present disclosure describes technologies for combination therapies using engineered T cells and a CD38 targeting compound. The technologies specifically address issues arising from combining therapeutics and generating and maximizing arising synergistic effects of the therapies while retaining and/or improving individually existing effects. The engineered T cells may include a CD38 expression modification offering protections against the CD38 targeting compound. The CD38 targeting compound may offer protection to the engineered T cells from NK cell-mediated rejection. Further, the combination therapies may target the same cells in a synergistic fashion. The technologies described herein may be embodied in methods of use, methods of manufacture, systems, engineered T cells, CD38 targeting compounds, kits, and other useful embodiments.

### CROSS-REFERENCE TO RELATED APPLICATIONS

This application claims the benefit of U.S. Provisional Application No. 63/554,420, filed on Feb. 16, 2024, the entire contents of which is hereby incorporated by reference in its entirety.

### FIELD

The present disclosure relates to the field of co-therapeutics, and more specifically, to co-therapeutics comprising T cells and CD38 antigen targeting compounds.

### SEQUENCE LISTING

The instant application contains a Sequence Listing which has been submitted electronically in. XML file format and is hereby incorporated by reference in its entirety. Said .XML copy, created on created on Feb. 3, 2025, is named K-1156-WO-PCT_SL.xml and is 23,721 bytes in size.

### BACKGROUND

A major challenge for implementing allogeneic cell therapies is immune rejection of donor cells by the recipient's adaptive and innate immune system. While evasion of cytotoxic T lymphocyte (CD8 T cell) responses can be accomplished by elimination of major histocompatibility complex (MHC) class I expression, this approach leads to a missing “self” signal that activates natural killer (NK) cell mediated rejection. Furthermore, even when MHC class I expression is unaltered, there is killer-cell immunoglobulin-like receptor (KIR) ligand mismatch between the donor and the recipient that promotes rejection of the donor cells by recipient NK cells. Therefore, strategies to reduce the abundance of NK cells in patients receiving allogeneic cell therapy are predicted to augment the therapeutic benefit of the allogenic cell therapy. The described technologies herein include strategies to reduce the abundance of NK cells in accordance with various embodiments, thereby, addressing this challenging problem in the field without having many of the disadvantages.

CD38 is a cell-surface receptor that is highly expressed by NK cells and plasma cells, and by other immune cell populations to various degrees. Patients treated with antibodies specific to CD38 demonstrate reduced number of NK cells in peripheral blood and as such preconditioning and/or co-administration of anti-CD38 antibodies in combination with an allogeneic cell therapy product may provide protection from NK cell-mediated rejection. Casneuf T et al., Deep immune profiling of patients treated with lenalidomide and dexamethasone with or without daratumumab. Leukemia. 2021 Fcb; 35 (2): 573-584. doi: 10.1038/s41375-020-0855-4. Epub 2020 May 26. PMID: 32457357; PMCID: PMC7862054. In addition to autologous cell therapy products, the described technologies herein include allogeneic cell therapy products that provide protection from NK cell-mediated rejection in accordance with various embodiments.

T cells express CD38 at low to intermediate levels in the absence of stimulation, but stimulation of the T cell receptor (TCR) or a chimeric antigen receptor (CAR) dramatically upregulates CD38 expression. This creates a difficult problem because stimulation is expected to occur in patients receiving adoptive T cell therapy. Increased levels of CD38 observed on activated T cells indicate that co-administration of CD38 targeting compounds (e.g., anti-CD38 antibodies) in combination with an adoptive T cell therapy product would not be a viable strategy due to antibody-dependent mechanisms including antibody dependent cellular cytotoxicity (ADCC), complement dependent cytotoxicity (CDC), and antibody dependent cellular phagocytosis (ADCP). The described technologies herein include strategies inhibit or eliminate T cell destruction through a CD38 pathway in accordance with various embodiments, thereby, addressing this challenging problem in the field.

What is needed is technology enabling a combination therapy using both engineered T cells and CD38 targeting compounds that eliminate the negative effects described above and herein while taking advantage of the synergistic effects (e.g., NK cell depletion leading to increased engineered T cell survival). The technology described herein solves these challenging problems and additional long existing problems in the field.

### SUMMARY

In various aspects, a cell or a population of cells comprising an engineered T cell comprising a CD38 expression modification is provided. In various embodiments, the CD38 expression modification comprises a CD38 gene knock-out. In various embodiments, the CD38 expression modification comprises a single amino acid substitution. In various embodiments, the single amino acid substitution occurs at an epitope specific for Daratumumab. In various embodiments, the single amino acid substitution occurs at an epitope specific for Isatuximab. In various embodiments, the single amino acid substitution may be mediated by a base editor. In various embodiments, the substitution replaces serine 274 with a phenylalanine. In various embodiments, the substitution replaces a threonine 116 with an alanine. In various embodiments, the engineered T cell comprises a CAR T cell. In various embodiments, the engineered T cell may be an allogeneic therapeutic. In various embodiments, the engineered T cell may be modified to target at least one of CD19, CD20, CLL-1, BCMA, EGFR, HER2, GPC3, or GD2.

In various aspects, a system for a combination therapy comprising a T cell and a CD38 targeting compound comprising an engineered T cell comprising a CD38 expression modification and a CD38 targeting compound is provided. In various embodiments, the CD38 targeting compound comprises an anti-CD38 antibody. In various embodiments, the CD38 targeting compound comprises at least one of a DARPin, a nano-body, a ligand trap, or a synthetic component. In various embodiments, the CD38 targeting compound comprises isatuximab. In various embodiments, the CD38 targeting compound comprises daratumumab. In various embodiments, the CD38 expression modification comprises a CD38 gene knock-out. In various embodiments, the engineered T cell is modified to target at least one of CD19, CD20, CLL-1, BCMA, EGFR, HER2, GPC3, or GD2.

In various aspects, a method of generating a cell therapy treatment comprising introducing a CD38 expression modification into an engineered T cell is provided. In various embodiments, the step of introducing further comprises knocking out a CD38 gene of the engineered T cell. In various embodiments, the step of knocking out the CD38 gene further comprises applying a base editor to the engineered T cell. In various embodiments, the base editor comprises at least one of a cytosine base editor (CBE) or an adenine base editor (ABE). In various embodiments, the engineered T cell comprises a CAR T cell. In various embodiments, the method further comprises engineering the engineered T cell to target at least one of CD19, CD20, CLL-1, BCMA, EGFR, HER2, GPC3, or GD2. In various embodiments, the method further comprises administering the engineered T cell to a patient and administering a CD38 targeting compound to the patient. In various embodiments, the CD38 targeting compound comprises an anti-CD38 antibody. In various embodiments, the CD38 targeting compound comprises at least one of a DARPin, a nano-body, a ligand trap, or a synthetic component.

### ADDITIONAL SUMMARY

In various aspects, a cell or a population of cells are described according to various embodiments. In various embodiments, a cell or population of cells may comprise an engineered T cell comprising a CD38 expression modification.

In various embodiments, the CD38 expression modification comprises a CD38 gene knock-out. In various embodiments, the gene knock-out is Cas9 mediated. In various embodiments, the Cas9 mediated gene knock-out uses a complex comprising a Cas9 molecule and a guide nucleotide sequence. In various embodiments, the nucleotide guide sequence comprises SEQ ID NO: 1. In various embodiments, the nucleotide guide sequence comprises of SEQ ID NO: 2. In various embodiments, the nucleotide guide sequence comprises of SEQ ID NO: 3. In various embodiments, the nucleotide guide sequence comprises of SEQ ID NO: 4.

In various embodiments, the gene knock-out is Cas 12 mediated.

In various embodiments, the gene knock-out is base editor mediated. In various embodiments, the base editor comprises a cytosine base editor (CBE). In various embodiments, the cytosine base editor comprises a cytidine base editor comprised of a Cas9 nickase fused to a cytidine deaminase derived from APOBEC1. In various embodiments, the base editor comprises an adenine base editor (ABE). In various embodiments, the base editor is complexed with a nucleotide guide sequence. In various embodiments, the nucleotide guide sequence comprises SEQ ID NO: 5. In various embodiments, the nucleotide guide sequence comprises of SEQ ID NO: 6. In various embodiments, the nucleotide guide sequence comprises of SEQ ID NO: 7. In various embodiments, the nucleotide guide sequence comprises of SEQ ID NO: 8. In various embodiments, the nucleotide guide sequence comprises of SEQ ID NO: 9. In various embodiments, the nucleotide guide sequence comprises of SEQ ID NO: 10.

In various embodiments, the CD38 expression modification comprises a CD38 gene knock-in. In various embodiments, the CD38 gene knock-in uses homology directed repair.

In various embodiments, the CD38 expression modification uses RNAi. In various embodiments, the CD38 expression modification uses anti-sense oligonucleotide (ASO).

In various embodiments, the CD38 expression modification uses epigenetic editing. In various embodiments, the epigenetic editing uses a TALEN. In various embodiments, the epigenetic editing uses a zinc finger nuclease. In various embodiments, the epigenetic editing uses CRISPRi. In various embodiments, the CD38 expression modification comprises a single amino acid substitution. In various embodiments, the single amino acid substitution occurs at an epitope specific for Daratumumab. In various embodiments, the single amino acid substitution occurs at an epitope specific for Isatuximab. In various embodiments, the single amino acid substitution is mediated by a base editor. In various embodiments, the substitution replaces serine 274. In various embodiments, the substitution replaces the serine 274 with a phenylalanine. In various embodiments, the base editor comprises a nucleotide guide sequence comprising SEQ ID NO: 11. In various embodiments, the substitution replaces threonine 116. In various embodiments, the substitution replaces the threonine 116 with an alanine. In various embodiments, the base editor comprises a nucleotide guide sequence comprising SEQ ID NO: 16. In various embodiments, the base editor comprises a nucleotide guide sequence comprising SEQ ID NO: 17. In various embodiments, the base editor comprises a nucleotide guide sequence comprising SEQ ID NO: 18. In various embodiments, the base editor comprises a nucleotide guide sequence comprising SEQ ID NO: 19. In various embodiments, the engineered T cell expresses a CD38 antigen nucleotide encoding sequence comprising SEQ ID NO: 21.

In various embodiments, the engineered T cell expresses a CD38 antigen protein molecule comprising SEQ ID NO: 23. In various embodiments, the engineered T cell expresses a CD38 antigen protein molecule comprising SEQ ID NO: 24.

In various embodiments, the engineered T cell is a CAR T cell. In various embodiments, the CAR T cell is an allogeneic therapeutic. In various embodiments, the CAR T cell is an autologous therapeutic.

In various embodiments, the engineered T cell targets CD20. In various embodiments, the engineered T cell targets CD19. In various embodiments, the engineered T cell targets CLL-1. In various embodiments, the engineered T cell targets BCMA. In various embodiments, the engineered T cell targets EGFR. In various embodiments, the CAR T cells target HER2. In various embodiments, the engineered T cell targets GPC3. In various embodiments, the engineered T cell targets GD2.

In various aspects, a system for a combination therapy comprising a T cell and a CD38 targeting compound is described according to various embodiments. In various embodiments, a system may comprise an engineered T cell comprising a CD38 expression modification. In various embodiments a system may comprise a CD38 targeting compound.

In various embodiments, the CD38 targeting compound comprises an antigen binding molecule having specificity for a CD38 antigen. In various embodiments, the antigen binding molecule comprises an anti-CD38 antibody. In various embodiments, the antigen binding molecule comprises a DARPin. In various embodiments, the antigen binding molecule comprises a nano-body. In various embodiments, the antigen binding molecule comprises a ligand trap. In various embodiments, the antigen binding molecule comprises a synthetic component. In various embodiments, the CD38 targeting compound comprises isatuximab. In various embodiments, the CD38 targeting compound comprises daratumumab. In various embodiments, the CD38 targeting compound comprises MOR202. In various embodiments, the CD38 targeting compound comprises TAK079.

In various embodiments, the CD38 expression modification comprises a CD38 gene knock-out. In various embodiments, the gene knock-out is Cas9 mediated. In various embodiments, the Cas9 mediated gene knock-out uses a complex comprising a Cas9 molecule and a guide nucleotide sequence. In various embodiments, the nucleotide guide sequence comprises SEQ ID NO: 1. In various embodiments, the nucleotide guide sequence comprises of SEQ ID NO: embodiments, the nucleotide guide sequence comprises SEQ ID NO: 4. In various embodiments, the gene knock-out is Cas12 mediated.

In various embodiments, the gene knock-out is base editor mediated. In various embodiments, the base editor comprises a cytosine base editor (CBE). In various embodiments, the cytosine base editor comprises a cytidine base editor comprised of a Cas9 nickase fused to a cytidine deaminase derived from APOBEC1. In various embodiments, the base editor comprises an adenine base editor (ABE). In various embodiments, the base editor is complexed with a nucleotide guide sequence. In various embodiments, the nucleotide guide sequence comprises SEQ ID NO: 5. In various embodiments, the nucleotide guide sequence comprises SEQ ID NO: 6. In various embodiments, the nucleotide guide sequence comprises SEQ ID NO: 7. In various embodiments, the nucleotide guide sequence comprises SEQ ID NO: 8. In various embodiments, the nucleotide guide sequence comprises SEQ ID NO: 9. In various embodiments, the nucleotide guide sequence comprises SEQ ID NO: 10.

In various embodiments, the system comprises a CD38 gene knock-in. In various embodiments, the CD38 gene knock-in uses homology directed repair. In various embodiments, the CD38 expression modification uses RNAi. In various embodiments, the CD38 expression modification uses anti-sense oligonucleotide (ASO).

In various embodiments, the CD38 expression modification uses epigenetic editing. In various embodiments, the epigenetic editing uses a TALEN. In various embodiments, the epigenetic editing uses a zinc finger nuclease. In various embodiments, the epigenetic editing uses CRISPRi. In various embodiments, the CD38 expression modification comprises a single amino acid substitution. In various embodiments, the single amino acid substitution occurs at an epitope specific for Daratumumab. In various embodiments, the single amino acid substitution occurs at an epitope specific for Isatuximab.

In various embodiments, the single amino acid substitution is mediated by a base editor. In various embodiments, the substitution replaces serine 274. In various embodiments, the substitution replaces the serine 274 with a phenylalanine. In various embodiments, the base editor comprises a nucleotide guide sequence comprises SEQ ID NO: 11. In various embodiments, the substitution replaces threonine 116. In various embodiments, the substitution replaces the threonine 116 with an alanine. In various embodiments, the base editor comprises a nucleotide guide sequence comprises SEQ ID NO: 16. In various embodiments, the base editor comprises a nucleotide guide sequence comprises SEQ ID NO: 17. In various embodiments, the base editor comprises a nucleotide guide sequence comprises SEQ ID NO: 18. In various embodiments, the base editor comprises a nucleotide guide sequence comprises SEQ ID NO: 19. In various embodiments, the engineered T cell expresses a CD38 antigen nucleotide encoding sequence comprises SEQ ID NO: 21.

In various embodiments, the engineered T cell expresses a CD38 antigen protein molecule comprises SEQ ID NO: 23. In various embodiments, the engineered T cell expresses a CD38 antigen protein molecule comprises SEQ ID NO: 24.

In various embodiments, the engineered T cell is a CAR T cell. In various embodiments, the system is an allogeneic therapeutic. In various embodiments, the system is an autologous therapeutic. In various embodiments, the engineered T cell targets CD20. In various embodiments, the engineered T cell targets CD19. In various embodiments, the engineered T cell targets CLL-1. In various embodiments, the engineered T cell targets BCMA. In various embodiments, the engineered T cell targets EGFR. In various embodiments, the CAR T cells target HER2. In various embodiments, the engineered T cell targets GPC3. In various embodiments, the engineered T cell targets GD2.

In various aspects, a method of generating a cell therapy treatment is described. In various embodiments, the method comprises introducing a CD38 expression modification into an engineered T cell. In various embodiments, the method may comprise activating the engineered T cell. In various embodiments, the method may comprise expanding the engineered T cell. In various embodiments, the method may comprise transducing the engineered T cell. In various embodiments, the method may comprise incubating the T cell with a lentiviral vector.

In various embodiments, the CD38 expression modification comprises a CD38 gene knock-out. In various embodiments, the gene knock-out is Cas9 mediated. In various embodiments, the Cas9 mediated gene knock-out uses a complex comprising a Cas9 molecule and a guide nucleotide sequence. In various embodiments, the nucleotide guide sequence comprises SEQ ID NO: 1. In various embodiments, the nucleotide guide sequence comprises SEQ ID NO: 2. In various embodiments, the nucleotide guide sequence comprises SEQ ID NO: 3. In various embodiments, the nucleotide guide sequence comprises SEQ ID NO: 4.

In various embodiments, the gene knock-out is Cas 12 mediated.

In various embodiments, the gene knock-out is base editor mediated. In various embodiments, the base editor comprises a cytosine base editor (CBE). In various embodiments, the cytosine base editor comprises a cytidine base editor comprised of a Cas9 nickase fused to a cytidine deaminase derived from APOBEC1. In various embodiments, the base editor comprises an adenine base editor (ABE). In various embodiments, the base editor is complexed with a nucleotide guide sequence. In various embodiments, the nucleotide guide sequence comprises SEQ ID NO: 5. In various embodiments, the nucleotide guide sequence comprises SEQ ID NO: 6. In various embodiments, the nucleotide guide sequence comprises SEQ ID NO: 7. In various embodiments, the nucleotide guide sequence comprises SEQ ID NO: 8. In various embodiments, the nucleotide guide sequence comprises SEQ ID NO: 9. In various embodiments, the nucleotide guide sequence comprises SEQ ID NO: 10.

In various embodiments, the method comprises a CD38 gene knock-in. In various embodiments, the CD38 gene knock-in uses homology directed repair.

In various embodiments, the CD38 expression modification uses RNAi. In various embodiments, the CD38 expression modification uses anti-sense oligonucleotide (ASO).

In various embodiments, the CD38 expression modification uses epigenetic editing. In various embodiments, the epigenetic editing uses a TALEN. In various embodiments, the epigenetic editing uses a zinc finger nuclease. In various embodiments, the epigenetic editing uses CRISPRi. In various embodiments, the CD38 expression modification comprises a single amino acid substitution. In various embodiments, the single amino acid substitution occurs at an epitope specific for Daratumumab. In various embodiments, the single amino acid substitution occurs at an epitope specific for Isatuximab. In various embodiments, the single amino acid substitution is mediated by a base editor. In various embodiments, the substitution replaces serine 274. In various embodiments, the substitution replaces the serine 274 with a phenylalanine. In various embodiments, the base editor comprises a nucleotide guide sequence comprising SEQ ID NO: 11. In various embodiments, the substitution replaces threonine 116. In various embodiments, the substitution replaces the threonine 116 with an alanine. In various embodiments, the base editor comprises a nucleotide guide sequence comprises SEQ ID NO: 16. In various embodiments, the base editor comprises a nucleotide guide sequence comprises SEQ ID NO: 17. In various embodiments, the base editor comprises a nucleotide guide sequence comprises SEQ ID NO: 18. In various embodiments, the base editor comprises a nucleotide guide sequence comprises SEQ ID NO: 19.

In various embodiments, the engineered T cell expresses a CD38 antigen nucleotide encoding sequence comprises SEQ ID NO: 21. In various embodiments, the engineered T cell expresses a CD38 antigen protein molecule comprises SEQ ID NO: 23. In various embodiments, the engineered T cell expresses a CD38 antigen protein molecule comprises SEQ ID NO: 24.

In various embodiments, the engineered T cell is a CAR T cell. In various embodiments, the system is an allogeneic therapeutic. In various embodiments, the system is an autologous therapeutic. In various embodiments, the engineered T cell targets CD20. In various embodiments, the engineered T cell targets CD19. In various embodiments, the engineered T cell targets CLL-1. In various embodiments, the engineered T cell targets BCMA. In various embodiments, the engineered T cell targets EGFR. In various embodiments, the CAR T cells target HER2. In various embodiments, the engineered T cell targets GPC3. In various embodiments, the engineered T cell targets GD2.

In various embodiments, the method comprises combining the CAR T cells with a CD38 targeting compound to generate a cell therapy product.

In various embodiments, the CD38 targeting compound comprises an antigen binding molecule having specificity for a CD38 antigen. In various embodiments, the antigen binding molecule comprises an anti-CD38 antibody. In various embodiments, the antigen binding molecule comprises a DARPin. In various embodiments, the antigen binding molecule comprises a nano-body. In various embodiments, the antigen binding molecule comprises a ligand trap. In various embodiments, the antigen binding molecule comprises a synthetic component. In various embodiments, the CD38 targeting compound comprises isatuximab. In various embodiments, the CD38 targeting compound comprises daratumumab. In various embodiments, the CD38 targeting compound comprises MOR202. In various embodiments, the CD38 targeting compound comprises TAK079.

In various aspects, a method of treating a patient comprising a T cell and a CD38 targeting compound is described. In various embodiments, the method may comprise administering an engineered T cell comprising a CD38 expression modification to a patient. In various embodiments, the method may comprise administering a CD38 targeting compound to the patient.

In various embodiments, the CD38 targeting compound comprises an antigen binding molecule having specificity for a CD38 antigen. In various embodiments, the antigen binding molecule comprises an anti-CD38 antibody. In various embodiments, the antigen binding molecule comprises a DARPin. In various embodiments, the antigen binding molecule comprises a nano-body. In various embodiments, the antigen binding molecule comprises a ligand trap. In various embodiments, the antigen binding molecule comprises a synthetic component.

In various embodiments, the CD38 targeting compound comprises Isatuximab. In various embodiments, the CD38 targeting compound comprises daratumubmab. In various embodiments, the CD38 targeting compound comprises MOR202. In various embodiments, the CD38 targeting compound comprises TAK079.

In various embodiments, the CD38 expression modification comprises a CD38 gene knock-out. In various embodiments, the gene knock-out is Cas9 mediated. In various embodiments, the Cas9 mediated gene knock-out uses a complex comprising a Cas9 molecule and a guide nucleotide sequence. In various embodiments, the nucleotide guide sequence comprises SEQ ID NO: 1. In various embodiments, the nucleotide guide sequence comprises SEQ ID NO: embodiments, the nucleotide guide sequence comprises SEQ ID NO: 4.

In various embodiments, the gene knock-out is Cas 12 mediated.

In various embodiments, the gene knock-out is base editor mediated. In various embodiments, the base editor comprises a cytosine base editor (CBE). In various embodiments, the cytosine base editor comprises a cytidine base editor comprised of a Cas9 nickase fused to a cytidine deaminase derived from APOBEC1. In various embodiments, the base editor comprises an adenine base editor (ABE). In various embodiments, the base editor is complexed with a nucleotide guide sequence. In various embodiments, the nucleotide guide sequence comprises SEQ ID NO: 5. In various embodiments, the nucleotide guide sequence comprises SEQ ID NO: 6. In various embodiments, the nucleotide guide sequence comprises SEQ ID NO: 7. In various embodiments, the nucleotide guide comprises of SEQ ID NO: 8. In various embodiments, the nucleotide guide sequence comprises SEQ ID NO: 9. In various embodiments, the nucleotide guide sequence comprises SEQ ID NO: 10.

In various embodiments, the method comprises a CD38 gene knock-in. In various embodiments, the CD38 gene knock-in uses homology directed repair.

In various embodiments, the CD38 expression modification uses RNAi. In various embodiments, the CD38 expression modification uses anti-sense oligonucleotide (ASO).

In various embodiments, the CD38 expression modification uses epigenetic editing. In various embodiments, the epigenetic editing uses a TALEN. In various embodiments, the epigenetic editing uses a zinc finger nuclease. In various embodiments, the epigenetic editing uses CRISPRi. In various embodiments, the CD38 expression modification comprises a single amino acid substitution. In various embodiments, the single amino acid substitution occurs at an epitope specific for Daratumumab. In various embodiments, the single amino acid substitution occurs at an epitope specific for Isatuximab. In various embodiments, the single amino acid substitution is mediated by a base editor. In various embodiments, the substitution replaces serine 274. In various embodiments, the substitution replaces the serine 274 with a phenylalanine. In various embodiments, the base editor comprises a nucleotide guide sequence comprising SEQ ID NO: 11. In various embodiments, the substitution replaces threonine 116. In various embodiments, the substitution replaces the threonine 116 with an alanine. In various embodiments, the base editor comprises a nucleotide guide sequence comprising SEQ ID NO: 16. In various embodiments, the base editor comprises a nucleotide guide sequence comprising SEQ ID NO: 17. In various embodiments, the base editor comprises a nucleotide guide sequence comprising SEQ ID NO: 18. In various embodiments, the base editor comprises a nucleotide guide sequence comprising SEQ ID NO: 19.

In various embodiments, the engineered T cell expresses a CD38 antigen nucleotide encoding a sequence comprising SEQ ID NO: 21. In various embodiments, the engineered T cell expresses a CD38 antigen protein molecule comprising SEQ ID NO: 23. In various embodiments, the engineered T cell expresses a CD38 antigen protein molecule comprising SEQ ID NO: 24.

In various embodiments, the engineered T cell is a CAR T cell. In various embodiments, the method is an allogeneic therapeutic. In various embodiments, the method is an autologous therapeutic.

In various embodiments, the engineered T cell targets CD20. In various embodiments, the engineered T cell targets CD19. In various embodiments, the engineered T cell targets CLL-1. In various embodiments, the engineered T cell targets BCMA. In various embodiments, the engineered T cell targets EGFR. In various embodiments, the CAR T cells target HER2. In various embodiments, the engineered T cell targets GPC3. In various embodiments, the engineered T cell targets GD2.

In various aspects, a kit for a combination therapy using a T cell and a CD38 targeting compound is described. In various embodiments, an engineered T cell comprising a CD38 expression modification.

In various embodiments, the kit may comprise a CD38 targeting compound. In various embodiments, the CD38 targeting compound comprises an antigen binding molecule having specificity for a CD38 antigen. In various embodiments, the antigen binding molecule comprises an anti-CD38 antibody. In various embodiments, the antigen binding molecule comprises a DARPin. In various embodiments, the antigen binding molecule comprises a nano-body. In various embodiments, the antigen binding molecule comprises a ligand trap. In various embodiments, the antigen binding molecule comprises a synthetic component. In various embodiments, the CD38 targeting compound comprises Isatuximab. In various embodiments, the CD38 targeting compound comprises daratumubmab. In various embodiments, the CD38 targeting compound comprises MOR202. In various embodiments, the CD38 targeting compound comprises TAK079.

In various embodiments, the CD38 expression modification comprises a CD38 gene knock-out. In various embodiments, the gene knock-out is Cas9 mediated. In various embodiments, the Cas9 mediated gene knock-out uses a complex comprising a Cas9 molecule and a guide nucleotide sequence. In various embodiments, the nucleotide guide sequence comprises SEQ ID NO: 1. In various embodiments, the nucleotide guide sequence comprises SEQ ID NO: 2. In various embodiments, the nucleotide guide sequence comprises SEQ ID NO: 3. In various embodiments, the nucleotide guide sequence comprises SEQ ID NO: 4.

In various embodiments, the gene knock-out is Cas 12 mediated.

In various embodiments, the gene knock-out is base editor mediated. In various embodiments, the base editor comprises a cytosine base editor (CBE). In various embodiments, the cytosine base editor comprises a cytidine base editor comprised of a Cas9 nickase fused to a cytidine deaminase derived from APOBEC1. In various embodiments, the base editor comprises an adenine base editor (ABE). In various embodiments, the base editor is complexed with a nucleotide guide sequence. In various embodiments, the nucleotide guide sequence comprises SEQ ID NO: 5. In various embodiments, the nucleotide guide sequence comprises SEQ ID NO: 6. In various embodiments, the nucleotide guide sequence comprises SEQ ID NO: 7. In various embodiments, the nucleotide guide sequence comprises SEQ ID NO: 8. In various embodiments, the nucleotide guide sequence comprises SEQ ID NO: 9. In various embodiments, the nucleotide guide sequence comprises SEQ ID NO: 10.

In various embodiments, the kit may comprise a CD38 gene knock-in. In various embodiments, the CD38 gene knock-in uses homology directed repair.

In various embodiments, the CD38 expression modification uses RNAi. In various embodiments, the CD38 expression modification uses anti-sense oligonucleotide (ASO).

In various embodiments, the CD38 expression modification uses epigenetic editing. In various embodiments, the epigenetic editing uses a TALEN. In various embodiments, the epigenetic editing uses a zinc finger nuclease. In various embodiments, the epigenetic editing uses CRISPRi. In various embodiments, the CD38 expression modification comprises a single amino acid substitution. In various embodiments, the single amino acid substitution occurs at an epitope specific for Daratumumab. In various embodiments, the single amino acid substitution occurs at an epitope specific for Isatuximab. In various embodiments, the single amino acid substitution is mediated by a base editor. In various embodiments, the substitution replaces serine 274. In various embodiments, the substitution replaces the serine 274 with a phenylalanine. In various embodiments, the base editor comprises a nucleotide guide sequence comprising SEQ ID NO: 11. In various embodiments, the substitution replaces threonine 116. In various embodiments, the substitution replaces the threonine 116 with an alanine. In various embodiments, the base editor comprises a nucleotide guide sequence comprising SEQ ID NO: 16. In various embodiments, the base editor comprises a nucleotide guide sequence comprising of SEQ ID NO: 17. In various embodiments, the base editor comprises a nucleotide guide sequence comprising SEQ ID NO: 18. In various embodiments, the base editor comprises a nucleotide guide sequence comprising of SEQ ID NO: 19.

In various embodiments, the engineered T cell expresses a CD38 antigen nucleotide encoding sequence comprising SEQ ID NO: 21. In various embodiments, the engineered T cell expresses a CD38 antigen protein molecule comprising SEQ ID NO: 23. In various embodiments, the engineered T cell expresses a CD38 antigen protein molecule comprising SEQ ID NO: 24.

In various embodiments, the engineered T cell is a CAR T cell. In various embodiments, the kit comprises an allogeneic therapeutic. In various embodiments, the kit is an autologous therapeutic.

In various embodiments, the engineered T cell targets CD20. In various embodiments, the engineered T cell targets CD19. In various embodiments, the engineered T cell targets CLL-1. In various embodiments, the engineered T cell targets BCMA. In various embodiments, the engineered T cell targets EGFR. In various embodiments, the CAR T cells target HER2. In various embodiments, the engineered T cell targets GPC3. In various embodiments, the engineered T cell targets GD2.

In various embodiments, any of the technology described herein may be for treatment of autoimmune diseases. In various embodiments, the technology described herein may target healthy memory B cells and/or healthy plasma cells.

### FIGURES

FIG. 1 illustrates a bar graph showing CD38 mRNA expression levels in various cell types.

FIG. 2 is experimental data showing an increase in CD38 expression on T cells after undergoing a stimulation procedure (e.g., activation). FIG. 2, panel A shows high levels of CD38 expression on T cells and natural killer cells (NK cells). FIG. 2, panel B shows an increase in CD38 expression levels on T cells and NK cells after undergoing a stimulation procedure compared to before undergoing the procedure.

FIG. 3 is a schematic diagram of a cell expressing components of the adenosine biosynthesis pathway. CD38 is the first enzyme in the pathway responsible for conversion of NAD+ to ADPR.

FIG. 4 is an illustration of an engineered T cell expressing chimeric antigen receptors (CARs) and CD38 antigens in accordance with various embodiments.

FIG. 5 is an illustration of a system in which a CD38 targeting compound is targeting CD38 antigens on both engineered T cells and a cancer cell in accordance with various embodiments.

FIG. 6 is an illustration of an engineered T cell expressing chimeric antigen receptors (CARs) without expression of CD38 antigens in accordance with various embodiments.

FIG. 7 is an illustration of an engineered T cell expressing chimeric antigen receptors (CARs) and CD38 antigens wherein the CD38 antigens have been modified in accordance with various embodiments.

FIG. 8 is an illustration of a system in which a CD38 targeting compound is targeting CD38 antigens of a cancer cell and engineered T cells are protected by a CD38 expression modification in accordance with various embodiments.

FIG. 9 is an illustration of a system in which CD38 targeting compound is targeting CD38 antigens on a cancer cell and engineered T cells are protected by a CD38 expression modification in accordance with various embodiments.

FIG. 10 illustrates a process for generating a cell therapy treatment in accordance with various embodiments.

FIG. 11 illustrates a process for treating a patient using a T cell and a CD38 targeting compound in accordance with various embodiments.

FIG. 12 is experimental data for deletion of CD38 in non-transduced human primary T cells using CRISPR/Cas9. FIG. 12, panel A is flow cytometric analysis data for resting T cells. FIG. 12, panel B, is flow cytometric analysis data for stimulated T cells.

FIG. 13 is experimental data for deletion of CD38 in non-transduced human primary T cells using a cytidine base editor. CBE; Cytidine Base Editor. FIG. 13, panel A shows CD38 expression assessed by flow cytometry. FIG. 13, panel B depicts quantification of CD38+ T cells as shown in FIG. 13, panel A.

FIG. 14 is experimental data for disruption of Daratumumab binding to non-transduced human primary T cells using a cytidine base editor and Cas9 knock-out by installing a point mutation to replace serine 274 with phenylalanine. FIG. 14, panel A is flow cytometry data showing CD38 expression. FIG. 14, panel B is flow cytometry data showing Daratumumab binding. FIG. 14, panel C shows quantitative Mean Fluorescence Intensity (MFI) data from a flow cytometry Daratumumab binding assay. Relative binding to the AAVSI control is shown above the bar. FIG. 14, panel D depicts Sanger sequencing analysis to determine base editing efficiency.

FIG. 15 is experimental data for deletion of CD38 in chimeric antigen receptor (CAR) T cells targeting CD19 and CD20. FIG. 15, panel A is flow cytometry data showing CD38 expression in CAR+ T cells expressing a bicistronic CD19 and CD20 CAR. FIG. 15, panel B is experimental data showing frequency of indels quantified by Sanger sequencing and Inference of CRISPR Editing (ICE).

FIG. 16 is experimental flow cytometry data showing loss of CD38 does not impact CAR expression.

FIG. 17 is experimental data showing loss of CD38 does not impact CAR-T cell expansion during manufacturing.

FIG. 18 is experimental data showing CD38-deficient CAR-T cells demonstrate cytotoxicity.

FIG. 19 is experimental data showing CD38-deficient CAR-T cells demonstrate cytokine release.

FIG. 20 is experimental data showing deletion of CD38 in CAR T cells targeting CD19, CLL1, and BCMA using CRISPR/Cas9. FIG. 20, panel A is flow cytometry data showing CAR expression in transduced CAR T cells targeting CD19, CLL1, and BCMA versus non-transduced cells. FIG. 20, panel B is flow cytometry expression data for CAR T cells targeting CD19, CLL1, and BCMA, and non-transduced cells. FIG. 20, panel C is CD38 editing efficiency data assessed by Sanger sequencing and ICE.

FIG. 21 is experimental data showing disruption of Isatuximab binding to non-transduced human primary T cells using an adenine base editor to disrupt the CD38 binding epitope, or Cas9 to knock-out CD38. FIG. 21, panel A is flow cytometry data showing CD38 expression (left), and flow cytometry data showing Isatuximab binding (right). FIG. 21, panel B is quantitative flow cytometry data showing Isatuximab binding. FIG. 21, panel C is data showing an analysis of Sanger sequencing to determine base editing efficiency using an adenine base editor to install a T116A point mutation into CD38.

FIG. 22 is experimental data showing disruption of Isatuximab binding to human primary CAR-T cells using an adenine base editor to disrupt the CD38 binding epitope, or Cas9 to knock-out CD38. FIG. 22, panel A shows Isatuximab binding that was assessed by flow cytometry. FIG. 22, panel B shows ADP-ribosyl cyclase enzymatic activity measured in cell lysates using a CD38 Activity Assay Kit.

FIG. 23 is a schematic depicting an in vivo study of daratumumab and CAR-T cell combination therapy using a disseminated xenograft mouse model of human Burkitt's B-cell lymphoma.

FIG. 24 is experimental data showing CD38 knock out CAR-T cells efficiently eliminate tumor cells in vivo with or without coadministration of daratumumab. FIG. 24, panel A shows tumor burden assessed by bioluminescent imaging. FIG. 24, panel B shows tumor burden in mice treated with control CAR-T cells (CD3E knockout) or CAR-T cells with both CD3E and CD38 knockout.

FIG. 25 is experimental data demonstrating CD38 knockout is required to prevent daratumumab-mediated depletion of CAR-T cells. FIG. 25, panel A shows human CD45+CD3-T cell counts from mice dosed with 5×10⁶ CAR-T cells. FIG. 25, panel B shows CAR+ T cell counts in mice dosed with 5×10⁶ CAR-T cells. FIG. 25, panel C shows Human CD45+CD3 T cells counts from mice dosed with 5×10⁶ CAR-T cells. FIG. 25, panel D shows CAR+ T cell counts in mice dosed with 1×10⁶ CAR-T cells. Mann Whitney test; ** P<0.001, *P<0.05.

FIG. 26 is experimental data showing efficient knock out of CD38 with adenine base editing. FIG. 26, panel A shows analysis of Sanger sequencing to determine base editing efficiency. FIG. 26, panel B shows quantification of A to G editing by sequencing, as shown in FIG. 26, panel A.

### DETAILED DESCRIPTION

The present disclosure provides insights and technologies useful in combination therapies. More specifically, a therapy that may use a CD38 targeting compound with a T cell including a CD38 expression modification in various embodiments described herein. Many issues relating to drug effectiveness and patient physiological response may be solved using the technologies described herein. Particularly, CD38 targeting compounds target a multitude of cells which may include another drug in the combination therapy (e.g., a T cell expressing an unmodified CD38 antigen). The systems and methods detailed herein enable a combination therapy where T cells comprising a CD38 expression modification are not targeted by CD38 targeting compounds, thereby, solving many issues involving drug effectiveness and patient physiological response.

Technologies for manufacturing, treating, systems, methods of use, devices, and/or kits for combination therapies are described in the accompanying description and figures. In the figures, numerous specific details are set forth to provide a thorough understanding of certain embodiments. A skilled artisan will appreciate that the systems and methods described herein may be used in a variety of ways and circumstances that are not limited to what is specifically detailed. Additionally, the skilled artisan will appreciate that certain embodiments may be practiced without these specific details. Furthermore, one skilled in the art can readily appreciate that the specific sequences in which methods are presented and performed are illustrative and it is contemplated that the sequences can be varied and still remain within the spirit and scope of certain embodiments.

While the present teachings are described in conjunction with various embodiments, it is not intended that the present teachings be limited to such embodiments. On the contrary, the present teachings encompass various alternatives, modifications, and equivalents, as will be appreciated by those skilled in the art.

### 1. Definitions

In order that the present disclosure may be more readily understood, certain terms are first defined. As used in this application, except as otherwise expressly provided herein, each of the following terms shall have the meaning set forth below. Additional definitions are set forth throughout the application. The headings provided herein are not limitations of the various aspects of the disclosure, which aspects should be understood by reference to the specification as a whole.

As used herein, the term the terms “a” and “an” are used per standard convention and mean one or more, unless context dictates otherwise.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. “About” or “comprising essentially of” can mean a range of up to 10% (i.e., +10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Système International de Unites (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2nd cd., (2001), CRC Press; “The Dictionary of Cell & Molecular Biology”, 5th ed., (2013), Academic Press; and “The Oxford Dictionary Of Biochemistry And Molecular Biology”, Cammack et al. cds., 2nd ed, (2006), Oxford University Press, provide those of skill in the art with a general dictionary for many of the terms used in this disclosure.

As used herein, the term “and/or” is to be understood as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or,” as used in a phrase such as ‘A, B, and/or C’” is intended to encompass each of the following aspects: A, B, and C; A, B, or C; A or C; A or B; B or C; A and C; A and B; B and C; A (alone); B (alone); and C (alone).

As used herein, the term the use of the alternative (e.g., “or”) should be understood to mean either one, both, or any combination thereof of the alternatives.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided.

As used throughout, the term “likely” refers to having a higher probability of occurring than not, or alternatively, of having a higher probability of occurring versus a predetermined control of average. By way of non-limiting example, a patient likely to experience toxicity following a cell therapy refers to that patient having a higher probability of experiencing toxicity than not. Alternatively, a patient likely to experience toxicity following a cell therapy refers to that patient having a higher statistical chance of experiencing toxicity as compared to the average occurrence of toxicity in a patient population treated with the cell therapy. One of ordinary skill in the art would recognize additional definitions in addition to the aforementioned.

As used herein, the terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

As used herein, the terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

As used herein, the terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions. Similarly, the term “increasing” indicates any change that is higher than the original value. “Increasing,” “higher,” and “lower” are relative terms, requiring a comparison between pre- and post-measurements and/or between reference standards. In some embodiments, the reference values are obtained from those of a general population, which could be a general population of patients. In some embodiments, the reference values come quartile analysis of a general patient population.

As used herein, the term “administering” may refer to the physical introduction of an agent to a subject, such as a modified T cell and/or a CD38 therapy as disclosed herein, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

As used herein, the term “antibody” (Ab) includes, without limitation, a glycoprotein immunoglobulin which binds specifically to an antigen. Antibodies described in the present application may include antibodies for targeting CD38 antigens. In general, an antibody can comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, C_{H}1, C_{H}2 and C_{H}3. Each light chain comprises a light chain variable region (abbreviated herein as VL) and a light chain constant region. The light chain constant region is comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (Clq) of the classical complement system. In general, human antibodies are approximately 150 kD tetrameric agents comprised of two identical heavy (H) chain polypeptides (about 50 kD each) and two identical light (L) chain polypeptides (about 25 kD each) that associate with each other into what is commonly referred to as a “Y-shaped” structure. The heavy and light chains are linked or connected to one another by a single disulfide bond; two other disulfide bonds connect the heavy chain hinge regions to one another, so that the dimers are connected to one another and the tetramer is formed. Naturally-produced antibodies are also glycosylated, e.g., on the C_{H}2 domain.

As used herein, the term “human antibody” may refer to antibodies having variable and constant domain sequences generated, assembled, or derived from human immunoglobulin sequences, or sequences indistinguishable therefrom in accordance with various embodiments. In some embodiments, antibodies (or antibody components) may be considered to be “human” even though their amino acid sequences comprise residues or elements not encoded by human germline immunoglobulin sequences (e.g., variations introduced by in vitro random or site-specific mutagenesis or introduced by in vivo somatic mutation). The term “humanized” is intended to comprise antibodies having a variable domain with a sequence derived from a variable domain of a non-human species (e.g., a mouse), modified to be more similar to a human germline encoded sequence. In some embodiments, a “humanized” antibody comprises one or more framework domains having substantially the amino acid sequence of a human framework domain, and one or more complementary determining regions having substantially the amino acid sequence as that of a non-human antibody. In some embodiments, a humanized antibody comprises at least a portion of an immunoglobulin constant region (Fc), generally that of a human immunoglobulin constant domain. In some embodiments, a humanized antibodies may comprise a C_{H}1, hinge, C_{H}2, C_{H}3, and, optionally, a C_{H}4 region of a human heavy chain constant domain.

Antibodies may include, for example, monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab fragments, F(ab’)₂ fragments, disulfide-linked Fvs (sdFv), anti-idiotypic (anti-ld) antibodies (including, e.g., anti-anti-ld antibodies), minibodies, domain antibodies, synthetic antibodies (sometimes referred to herein as “antibody mimetics”), and antigen binding fragments of any of the above. In certain embodiments, antibodies described herein refer to polyclonal antibody populations. Antibodies may also comprise, for example, Fab′ fragments, Fd′ fragments, Fd fragments, isolated CDRs, single chain Fvs, polypeptide-Fc fusions, single domain antibodies (e.g., shark single domain antibodies such as IgNAR or fragments thereof, and human heavy-chain antibodies (UniAbs)), camelid antibodies, single chain or Tandem diabodies (TandAb®), Anticalins®, Nanobodies® minibodies, BiTERs, ankyrin repeat proteins or DARPINS®, Avimers®, DARTs, TCR-like antibodies, Adnectins®, Affilins®, Trans-bodies®, Affibodies®, TrimerX®, MicroProteins, Fynomers®, Centyrins®, and KALBITOR®S.

An immunoglobulin may be derived from any of the commonly known isotypes, including but not limited to IgA, secretory IgA, IgG, IgE and IgM. IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized by recombinant methods to reduce its immunogenicity in man. Where not expressly stated, and unless the context indicates otherwise, the term “antibody” also includes an antigen binding fragment or an antigen-binding portion of any of the aforementioned immunoglobulins, and includes a monovalent and a divalent fragment or portion, and a single chain Ab.

As used herein, the terms “antigen binding molecule,” “antigen binding portion,” “antigen binding fragment,” “antibody fragment” or “antigen binding domain” may refer to any molecule that comprises the antigen binding parts of the molecule. In an example an antigen binding molecule is an antibody, or portion thereof, such as an scFv. In an example an antigen biding molecule is a portion of a TCR that binds an antigen and may be the antigen binding portion of the TCR alpha chain and/or the antigen binding portion of a TCR alpha chain. Non-limiting examples of antigen binding molecule targets may include CD20, CD19, CLL-1, BCMA, EGFR, HER2, GPC3, GD2, or any combination thereof. In an example, an antigen biding molecule may be a portion of NKG2D that binds an NKG2D ligand. An antigen binding molecule can include the antigenic complementarity determining regions (CDRs). Examples of antibody fragments include, but are not limited to, Fab, Fab′, F(ab′) 2, and Fv fragments, dAb, linear antibodies, scFv antibodies, and multispecific antibodies formed from antigen binding molecules. Peptibodies (i.e., Fc fusion molecules comprising peptide binding domains) are additional non-limiting examples of suitable antigen binding molecules. In some embodiments, the antigen binding molecule binds to an antigen on a tumor cell. In some embodiments, the antigen binding molecule may bind to an antigen on a cell involved in a hyperproliferative disease or to a viral or bacterial antigen. In various embodiments, an antigen binding molecule may include a chimeric antigen receptor (CAR) or an engineered T cell receptor (TCR). In certain embodiments, the antigen binding molecule or domain may include an antibody fragment that may specifically bind to the antigen, including one or more of the complementarity determining regions (CDRs) thereof. In further embodiments, the antigen binding molecule may include a single chain variable fragment (scFv).

In various embodiments, an antigen binding molecule may comprise an antibody memetics. Antibody memetics may include compounds similar to antibodies capable of binding to antigens. In various embodiments, antibody memetics may not be structurally related to antibodies. In various embodiments, antibody mimetics may include an artificial peptide or protein. In some embodiments, an antibody mimetic may include one or more nucleic acids. In various embodiments, a molar mass of an antibody memetic may range from about 3 to 20 kDa. Non-limiting examples of memetics may include affibody molecules, affilins, affimers, affitins, alphabodies, anticalins, avimers, DARPins, fynomers, gastrobodies, kunitz domain peptides, monobodies, nanoCLAMPs, optimers, repebodies, pronectin, centyrins, and obodies. In some circumstances, antibody mimetics may improve solubility, tissue penetration, and stability when exposed to heat and enzymes

In some instances, a CDR may be substantially identical to one found in a reference antibody (e.g., an antibody of the present disclosure) and/or the sequence of a CDR provided in the present disclosure. In some embodiments, a CDR is substantially identical to a reference CDR in that it is either identical in sequence or contains between 1, 2, 3, 4, or 5 (e.g., 1-5) amino acid substitutions as compared with the reference CDR. In some embodiments a CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR (e.g., 85-90%, 85-95%, 85-100%, 90-95%, 90-100%, or 95-100%). In some embodiments a CDR is substantially identical to a reference CDR in that it shows at least 96%, 96%, 97%, 98%, 99%, or 100% sequence identity with the reference CDR. In some embodiments a CDR is substantially identical to a reference CDR in that one amino acid within the CDR is deleted, added, or substituted as compared with the reference CDR while the CDR has an amino acid sequence that is otherwise identical with that of the reference CDR. In some embodiments a CDR is substantially identical to a reference CDR in that 2, 3, 4, or 5 (e.g., 2-5) amino acids within the CDR are deleted, added, or substituted as compared with the reference CDR while the CDR has an amino acid sequence that is otherwise identical to the reference CDR. In various embodiments, an antigen binding fragment binds a same antigen as a reference antibody. In various embodiments, an antigen binding fragment cross-competes with the reference antibody, for example, binding to substantially the same or identical epitope as the reference antibody.

An antigen binding fragment may be produced by any means. For example, in some embodiments, an antigen binding fragment may be enzymatically or chemically produced by fragmentation of an intact antibody. In some embodiments, an antigen binding fragment may be recombinantly produced (such as by expression of an engineered nucleic acid sequence). In some embodiments, an antigen binding fragment may be wholly or partially synthetically produced. In some embodiments, an antigen binding fragment may have a length of at least about 50, 60, 70, 80, 90, 100, 110, 120, 130, 140, 150, 160, 170, 180, 190 amino acids or more; in some embodiments at least about 200 amino acids (e.g., 50-100, 50-150, 50-200, or 100-200 amino acids).

As used herein, the terms “variable region” or “variable domain” are used interchangeably. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In embodiments, the variable region is a primate (e.g., non-human primate) variable region. In certain embodiments, the variable region comprises rodent or murine CDRs and primate (e.g., non-human primate) framework regions (FRs).

As used herein, the terms “VL” and “VL domain” are used interchangeably to refer to the light chain variable region of an antibody or an antigen-binding molecule thereof.

The terms “VH” and “VH domain” are used interchangeably to refer to the heavy chain variable region of an antibody or an antigen-binding molecule thereof.

A number of definitions of the CDRs are commonly in use: Kabat numbering, Chothia numbering, AbM numbering, or contact numbering. The AbM definition is a compromise between the two used by Oxford Molecular's AbM antibody modelling software. The contact definition is based on an analysis of the available complex crystal structures.

TABLE 1
CDR Numbering

| Loop | Kabat | AbM | Chothia | Contact |
|---|---|---|---|---|
| L1 | L24--L34 | L24--L34 | L24--L34 | L30--L36 |
| L2 | L50--L56 | L50--L56 | L50--L56 | L46--L55 |
| L3 | L89--L97 | L89--L97 | L89--L97 | L89--L96 |
| H1 | H31--H35B | H26--H35B | H26--H32 . . . 34 | H30--H35B |
| | (Kabat | | | |
| | Numbering | | | |
| H1 | H31--H35 | H26--H35 | H26--H32 | H30--H35 |
| | (Chothia | | | |
| | Numbering) | | | |
| H2 | H50--H65 | H50--H58 | H52--H56 | H47--H58 |
| H3 | H95--H102 | H95--H102 | H95--H102 | H93--H101 |

As used herein, the term “Kabat numbering” and like terms are recognized in the art and refer to a system of numbering amino acid residues in the heavy and light chain variable regions of an antibody, or an antigen-binding molecule thereof. In certain aspects, the CDRs of an antibody can be determined according to the Kabat numbering system (see, e.g., Kabat EA & Wu TT (1971) Ann NY Acad Sci 190:382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In a specific embodiment, the CDRs of the antibodies described herein have been determined according to the Kabat numbering scheme.

In certain aspects, the CDRs of an antibody can be determined according to the Chothia numbering scheme, which refers to the location of immunoglobulin structural loops (see, e.g., Chothia C & Lesk AM, (1987), J Mol Biol 196:901-917; Al-Lazikani B et al., (1997) J Mol Biol 273:927-948; Chothia C et al., (1992) J Mol Biol 227:799-817; Tramontano A et al., (1990) J Mol Biol 215 (1): 175-82; and U.S. Pat. No. 7,709,226). Typically, when using the Kabat numbering convention, the Chothia CDR-HI loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-HI loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). In a specific embodiment, the CDRs of the antibodies described herein have been determined according to the Chothia numbering scheme.

As used herein, the terms “constant region” and “constant domain” are interchangeable and have a meaning common in the art. The constant region is an antibody portion, e.g., a carboxyl terminal portion of a light and/or heavy chain which is not directly involved in binding of an antibody to antigen, but which can exhibit various effector functions, such as interaction with the Fc receptor. The constant region of an immunoglobulin molecule generally has a more conserved amino acid sequence relative to an immunoglobulin variable domain.

As used herein, the terms “heavy chain” when used in reference to an antibody can refer to any distinct type, e.g., alpha (a), delta (8), epsilon (¿), gamma (Y) and mu (u), based on the amino acid sequence of the constant domain, which give rise to IgA, IgD, IgE, IgG and IgM classes of antibodies, respectively, including subclasses of IgG, e.g., IgG1, IgG2, IgG3 and IgG4.

As used herein, the terms “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain.

An “antigen” refers to a compound, composition, or substance that may stimulate the production of antibodies or a T cell response in a human or animal, including compositions (such as one that includes a tumor-specific protein) that are injected or absorbed into a human or animal. An antigen reacts with the products of specific humoral or cellular immunity, including those induced by heterologous antigens, such as the disclosed antigens. A “target antigen” or “target antigen of interest” is an antigen that is not substantially found on the surface of other normal (desired) cells and to which a binding domain of a TCR or CAR contemplated herein, is designed to bind. A person of skill in the art would readily understand that any macromolecule, including virtually all proteins or peptides, can serve as an antigen. An antigen can be endogenously expressed, i.e. expressed by genomic DNA, or can be recombinantly expressed. An antigen can be specific to a certain tissue, such as a cancer cell, or it can be broadly expressed. In addition, fragments of larger molecules can act as antigens. A “target” is any molecule bound by a binding motif, CAR, TCR or antigen binding agent, e.g., an antibody.

As used herein, the term “antigen-specific targeting region” (ASTR) may refer to the region of the CAR or TCR which targets specific antigens. In various embodiments, the targeting regions on the CAR or TCR may be extracellular. In various embodiments, the antigen-specific targeting regions comprise an antibody or a functional equivalent thereof or a fragment thereof or a derivative thereof and each of the targeting regions target a different antigen. In various embodiments, the targeting regions may comprise full length heavy chain, Fab fragments, single chain Fv (scFv) fragments, divalent single chain antibodies or diabodies, each of which are specific to the target antigen. There are, however, numerous alternatives, such as linked cytokines (which leads to recognition of cells bearing the cytokine receptor), affibodies, ligand binding domains from naturally occurring receptors soluble protein/peptide ligand for a receptor (for example on a tumor cell), peptides, and vaccines to prompt an immune response, which may each be used in various embodiments of this disclosure. In fact, almost any molecule that binds a given antigen with high affinity can be used as an antigen-specific targeting region, as will be appreciated by those of skill in the art.

As used herein, the term “apheresis material” may originate from blood. In various embodiments, patient derived blood may undergo an apheresis process to generate an apheresis material. In various embodiments, the apheresis material may include leukocytes. Additional examples of an apheresis material may include platelets, white blood cells, or any constituent of blood. In more specific examples, T cells may be collected from patients for later processing (e.g., T cell modification).

In various embodiments, an apheresis process may include collecting whole blood from a patient. In various embodiments, the whole blood may be subjected to a size separation protocol. A common size selection protocol may employ use of centrifuge to separate the whole blood components by their respective weights. For example, this technique may result in generation of separation layers including plasma, a buffy coat, and erythrocytes. In various embodiments, the resulting plasma layer may make up about 55% of the total volume. In various embodiments, the resulting erythrocyte layer may make up about 45% of the total volume. In various embodiments, the resulting buffy coat layer may make up about less than 1% of the total volume. In various embodiments, the buffy coat may include leukocytes and platelets which may be further processed to further separate components of the buffy coat. For example, T cells may be isolated for later modification using the techniques described herein and elsewhere.

As used herein, the term “autologous” may refer to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient. In various embodiments, the methods described herein may be incorporated into an autologous T cell manufacturing system and/or process.

As used herein, the term “allogeneic” may refer to any material derived from one individual which is then introduced to another individual of the same species, e.g., allogeneic T cell transplantation. In various embodiments, the methods described herein may be incorporated into an allogeneic T cell manufacturing system and/or process.

As used herein, the term “binding” generally refers to a non-covalent association between or among two or more entities. Direct binding involves physical contact between entities or moieties. “Indirect” binding involves physical interaction by way of physical contact with one or more intermediate entities. Binding between two or more entities may be assessed in any of a variety of contexts, e.g., where interacting entities or moieties are studied in isolation or in the context of more complex systems (e.g., while covalently or otherwise associated with a carrier entity and/or in a biological system such as a cell).

The terms “immunospecifically binds,” “immunospecifically recognizes,” “specifically binds,” and “specifically recognizes” are analogous terms in the context of antibodies and refer to molecules that bind to an antigen (e.g., epitope or immune complex) as such binding is understood by one skilled in the art. For example, a molecule that specifically binds to an antigen may bind to other peptides or polypeptides, generally with lower affinity as determined by, e.g., immunoassays, BIACORE®, KinExA 3000 instrument (Sapidyne Instruments, Boise, ID), or other assays known in the art. In a specific embodiment, molecules that specifically bind to an antigen bind to the antigen with a KA that is at least 2 logs, 2.5 logs, 3 logs, 4 logs or greater than the KA when the molecules bind to another antigen. Binding may comprise preferential association of a binding domain, antibody, or antigen binding system with a target of the binding domain, antibody, or antigen binding system as compared to association of the binding domain, antibody, or antigen binding system with an entity that is not the target (i.e. non-target). In some embodiments, a binding domain, antibody, or antigen binding system selectively binds a target if binding between the binding domain, antibody, or antigen binding system and the target is greater than 2-fold, greater than 5-fold, greater than 10-fold, 20-fold, 30-fold, 40-fold, 50-fold, 60-fold, 70-fold, 80-fold, 90-fold, or greater than 100-fold as compared with binding of the binding domain, antibody, or antigen binding system and a non-target. In various embodiments, a binding domain, antibody, or antigen binding system selectively binds a target if the binding affinity is less than about 10⁻⁵ M, less than about 10⁻⁶ M, less than about 10⁻⁷ M, less than about 10⁻⁸ M, or less than about 10⁻⁹ M.

In another embodiment, molecules that specifically bind to an antigen bind with a dissociation constant (K_{d}) of about 1×10⁻⁷ M. In some embodiments, the antigen binding molecule specifically binds an antigen with “high affinity” when the K_{d} is about 1×10⁻⁹ M to about 5×10⁻⁹ M. In some embodiments, the antigen binding molecule specifically binds an antigen with “very high affinity” when the K_{d} is 1×10⁻¹⁰ M to about 5×10⁻¹⁰ M. In one embodiment, the antigen binding molecule has a K_{d} of 10⁻⁹ M. In one embodiment, the off-rate is less than about 1×10⁻⁵.

In certain embodiments, provided herein is an antibody or an antigen binding molecule thereof that binds to the target human antigen with a 5%, 10%, 15%, 20%, 25%, 30%, 35%, 40%, 45%, 50%, 55%, 60%, 65%, 70% or higher affinity than to another species of the target antigen as measured by, e.g., a radioimmunoassay, surface plasmon resonance, or kinetic exclusion assay. In a specific embodiment, an antibody or an antigen binding molecule thereof described herein, which binds to a target human antigen, will bind to another species of the target antigen with less than 10%, 15%, or 20% of the binding of the antibody or an antigen binding molecule thereof to the human antigen as measured by, e.g., a radioimmunoassay, surface plasmon resonance, or kinetic exclusion assay.

As used herein the term “cancer” may refer to a broad group of various diseases characterized by the uncontrolled growth of abnormal cells in the body. The cell therapy products the T cell manufacturing processes described herein are designed to generate may be used to treat cancer among other things. Cancer may mean unregulated cell division and growth results in the formation of malignant tumors that invade neighboring tissues and may also metastasize to distant parts of the body through the lymphatic system or bloodstream. A “cancer” or “cancer tissue” may include a tumor. In this application, the term cancer is synonymous with malignancy. Examples of cancers that may be treated by the methods disclosed herein include, but are not limited to, cancers of the immune system including lymphoma, leukemia, myeloma, and other leukocyte malignancies. In various embodiments, the methods disclosed herein may be used to reduce the tumor size of a tumor derived from, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, multiple myeloma, Hodgkin's Discase, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T cell lymphoma, environmentally induced cancers including those induced by asbestos, other B cell malignancies, and combinations of said cancers. In some embodiments, the cancer is multiple myeloma. In some embodiments, the cancer is NHL. The particular cancer may be responsive to chemo- or radiation therapy or the cancer may be refractory. A refractory cancer refers to a cancer that is not amenable to surgical intervention and the cancer is either initially unresponsive to chemo- or radiation therapy or the cancer becomes unresponsive over time.

As used herein, the term “chimeric antigen receptor” or “CAR” may refer to a molecule engineered to comprise a binding domain and a means of activating immune cells (for example T cells such as naive T cells, central memory T cells, effector memory T cells, NK cells or combination thereof) upon antigen binding. CARs are also known as artificial T cell receptors, chimeric T cell receptors or chimeric immunoreceptors. In various embodiments, a CAR may comprise a binding domain, an extracellular domain, a transmembrane domain, one or more co-stimulatory domains, and an intracellular signaling domain. A T cell that has been genetically engineered to express a chimeric antigen receptor may be referred to as a CAR T cell. Similarly, an NK cell that has been genetically engineered to express a chimeric antigen receptor may be referred to as a CAR NK cell.

In various embodiments, the binding domain of a CAR may be followed by a “spacer,” or, “hinge,” which may refer to the region that moves the antigen binding domain away from the effector cell surface to enable proper cell/cell contact, antigen binding and activation (Patel et al., Gene Therapy, 1999; 6:412-419). The hinge region in a CAR is generally between the transmembrane (TM) and the binding domain. In certain embodiments, a hinge region may be an immunoglobulin hinge region and may be a wild type immunoglobulin hinge region or an altered wild type immunoglobulin hinge region, such as an Igg4 hinge. Other exemplary hinge regions used in the CARs described herein include the hinge region derived from the extracellular regions of type 1 membrane proteins such as CD8alpha, CD4, CD28 and CD7, which may be wild-type hinge regions from these molecules or may be altered.

As used herein, the term “cell therapy product” may refer to a therapy including viable cells. The viable cells may be administered to a patient. Administration may occur by injection, translation, or infusion. In various embodiments, the viable cells may include immune cells. In various embodiments, the immune cells may include T cells. In various embodiments, the T cells may include chimeric antigen receptors (CARs).

As used herein, the terms “combination therapy” and “co-therapy” are used interchangeable herein and may refer to those situations in which a patient is simultaneously exposed to two or more therapeutic regimens (e.g., two or more therapeutic moieties). In various embodiments, the two or more regimens may be administered simultaneously; in various embodiments, such regimens may be administered sequentially (e.g., all “doses” of a first regimen are administered prior to administration of any doses of a second regimen); in various embodiments, such agents may be administered in overlapping dosing regimens. In various embodiments, “administration” of combination therapy may involve administration of one or more agent(s) or modality(ies) to a patient receiving the other agent(s) or modality(ies) in the combination. For clarity, combination therapy does not require that individual agents be administered together in a single composition (or even necessarily at the same time), although in various embodiments, two or more agents, or active moieties thereof, may be administered together in a combination composition, or even in a combination compound (e.g., as part of a single chemical complex or covalent entity). A non-limiting example of a combination therapy may include a cell therapy product combined with use of another therapeutic agent. In various embodiments, the therapeutic agent may include an antibody therapy. In various embodiments, the therapeutic agent may include a monoclonal antibody. In various embodiments, the monoclonal antibody may target CD38 antigens. For example, a patient undergoing combination therapy may receive a CAR T cell therapy product as well as a product targeting CD38. Non-limiting examples of CD38 targeting therapies may include Daratumumab and Isatuximab. Non-limiting examples of the benefits of using combination therapy may include increased efficacy and safety.

As used herein, the terms “costimulatory signaling domain” and “costimulatory domain” are used interchangeably and may refer to the portion of the CAR comprising the intracellular domain of a costimulatory molecule. Costimulatory molecules are cell surface molecules other than antigen receptors or Fc receptors that provide a second signal required for efficient activation and function of T lymphocytes upon binding to antigen. Non-limiting examples of such co-stimulatory molecules may include CD27, CD28, 4-1 BB (CD137), OX40 (CD134), CD30, CD40, PD-1, ICOS (CD278), LFA-1, CD2, CD7, LIGHT, NKD2C, 2B4, CD137, DAP12, B7-H2 and a ligand that specifically binds CD83. A skilled artisan will appreciate that other costimulatory domains may be used with the CARs described herein. In various embodiments, the inclusion of one or more costimulatory signaling domains may enhance the efficacy and expansion of T cells and NK cells expressing CAR receptors. In various embodiments, he intracellular signaling and costimulatory signaling domains may be linked in any order in tandem to the carboxyl terminus of the transmembrane domain.

Although CARs engineered to contain a signaling domain from CD3 or FcRgamma have been shown to deliver a potent signal for T cell activation and effector function, they are not sufficient to elicit signals that promote T cell survival and expansion in the absence of a concomitant costimulatory signal. Other CARs containing a binding domain, a hinge, a transmembrane and the signaling domain derived from CD3zeta or FcRgamma together with one or more costimulatory signaling domains (e.g., intracellular costimulatory domains derived from 4-1BB, CD28, CD134 and CD278) may more effectively direct antitumor activity as well as increased cytokine secretion, lytic activity, survival and proliferation in CAR expressing T cells in vitro, and in animal models and cancer patients (Milone et al., Molecular Therapy, 2009; 17:1453-1464; Zhong et al., Molecular Therapy, 2010; 18:413-420; Carpenito et al., PNAS, 2009; 106:3360-3365).

A “costimulatory signal” may refer to a signal, which in combination with a primary signal, such as TCR/CD3 ligation, leads to a T cell response, such as, but not limited to, proliferation and/or upregulation or down regulation of key molecules.

A “costimulatory ligand” includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand can include, but is not limited to, 3/TR6, 4-1BB ligand, agonist or antibody that binds Toll ligand receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS-L), intercellular adhesion molecule (ICAM), ligand that specifically binds with B7-H3, lymphotoxin beta receptor, MHC class I chain-related protein A (MICA), MHC class I chain-related protein B (MICB), OX40 ligand, PD-L2, or programmed death (PD) L1. A co-stimulatory ligand includes, without limitation, an antibody that specifically binds with a co-stimulatory molecule present on a T cell, such as, but not limited to, 4-1BB, B7-H3, CD2, CD27, CD28, CD30, CD40, CD7, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

As used herein, the term “costimulatory molecule” may refer to a cognate binding partner on a T cell that specifically binds with a costimulatory ligand, thereby mediating a costimulatory response by the T cell, such as, but not limited to, proliferation. Costimulatory molecules include, but are not limited to, A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, 4-1BB/CD137, B7-H3, BAFFR, BLAME (SLAMF8), BTLA, CD 33, CD 45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CD1-1a, CD1-1b, CD1-1c, CD1-1d, CDS, CEACAM1, CRT AM, DAP-10, DNAMI (CD226), Fc gamma receptor, GADS, GITR, HVEM (LIGHTR), IA4, ICAM-1, ICAM-1, ICOS, Ig alpha (CD79a), IL2R beta, IL2R gamma, IL7R alpha, integrin, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, LIGHT, LIGHT (tumor necrosis factor superfamily member 14; TNFSF14), LTBR, Ly9 (CD229), lymphocyte function-associated antigen-1 (LFA-1 (CD11a/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Ly108), SLAMF7, SLP-76, TNF, TNFr, TNFR2, Toll ligand receptor, TRANCE/RANKL, VLA1, or VLA-6, or fragments, truncations, or combinations thereof.

As used herein, the term “conservative amino acid substitution” may mean a substation in which the amino acid residue is replaced with an amino acid residue having a similar side chain. Families of amino acid residues having side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In various embodiments, one or more amino acid residues within a CDR(s) or within a framework region(s) of an antibody or antigen-binding molecule thereof may be replaced with an amino acid residue with a similar side chain. In general, two sequences may be generally considered to be “substantially similar” if they contain a conservative amino acid substitution in corresponding positions. For example, certain amino acids are generally classified as “hydrophobic” or “hydrophilic” amino acids, and/or as having “polar” or “non-polar” side chains. Substitution of one amino acid for another of the same type may be considered a conservative substitution. Exemplary amino acid categorizations are summarized in Tables 2 and 3 below:

TABLE 2

| | 3- | 1- | | | Hydropathy |
|---|---|---|---|---|---|
| Amino Acid | Letter | Letter | Property | Property | Index |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

TABLE 3

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As used herein, the term “DNA binding molecule” may refer to a molecule that can bind to DNA. Such DNA binding molecule can be a polypeptide, a domain of a protein, a domain within a larger protein or a polynucleotide. In some embodiments, the polynucleotide is DNA, while in other embodiments, the polynucleotide is RNA. In some embodiments, the DNA binding molecule is a protein domain of a nuclease (e.g., the FokI domain), while in other embodiments, the DNA binding molecule is a guide RNA component of an RNA-guided nuclease (e.g., Cas 9, Cas 12 or Cfp1).

As used herein, the term “domain” may refer to a portion of an entity. In various embodiments, a “domain” may be associated with a structural and/or functional feature of the entity, e.g., so that, when the domain is physically separated from the rest of its parent entity, it substantially or entirely retains the structural and/or functional feature. In various embodiments, a domain may comprise a portion of an entity that, when separated from that (parent) entity and linked or connected with a different (recipient) entity, substantially retains and/or imparts on the recipient entity one or more structural and/or functional features, e.g., that characterized it in the parent entity. In some embodiments, a domain is a portion of a molecule (e.g., a small molecule, carbohydrate, lipid, nucleic acid, or polypeptide). In some embodiments, a domain is a section of a polypeptide; in some such embodiments, a domain is characterized by a structural element (e.g., an amino acid sequence or sequence domain, α-helix character, β-sheet character, coiled-coil character, random coil character, etc.), and/or by a functional feature (e.g., binding activity, enzymatic activity, folding activity, signaling activity, etc.).

As used herein, the term “dosage form” may refer to a physically discrete unit of an active agent (e.g., an antigen binding system or antibody) for administration to a patient. In various embodiments, each subunit contains a predetermined quantity of active agent. In various embodiments, such quantity may be a unit dosage amount (or a whole fraction thereof) appropriate for administration in accordance with a dosing regimen that has been determined to correlate with a desired or beneficial outcome when administered to a relevant population. The total amount of a therapeutic composition or agent administered to a patient may be determined by one or more medical practitioners and may involve administration of more than one dosage forms.

As used herein, the term “dosing regimen” may refer to a set of one or more unit doses that are administered individually to a patient. In various embodiments, a therapeutic agent may include a recommended dosing regimen. In various embodiments, the dosing regimen may involve one or more doses. In various embodiments, a dosing regimen may comprise a plurality of doses each of which may be separated in time from other doses. In various embodiments, a dosing regimen may comprise a plurality of doses and consecutive doses are separated from one another by time periods of equal length. In various embodiments, a dosing regimen may comprise a plurality of doses and consecutive doses may be separated from one another by time periods of at least two different lengths. In various embodiments, all doses within a dosing regimen may be of the same unit dose amount. In various embodiments, different doses within a dosing regimen may be of different amounts. In various embodiments, a dosing regimen may comprise a first dose in a first dose amount, followed by one or more additional doses in a second dose amount different from the first dose amount. In various embodiments, a dosing regimen may be periodically adjusted to achieve a desired or beneficial outcome.

As used herein, the term “epitope” may refer to a localized region of an antigen to which an antibody can specifically bind. In various embodiments, an epitope may include a contiguous amino acid of a polypeptide (linear or contiguous epitope). In various embodiments, an epitope may include an amino acid or polypeptide that comes together from two or more non-contiguous regions (conformational, non-linear, discontinuous, or non-contiguous epitope). In various embodiments, the epitope to which an antibody binds may be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé R et al., (1994) Acta Crystallogr D Biol Crystallogr 50 (Pt 4): 339-350; McPherson A (1990) Eur J Biochem 189:1-23; Chayen NE (1997) Structure 5:1269-1274; McPherson A (1976) J Biol Chem 251:6300-6303). Antibody:antigen crystals may be studied using well known X-ray diffraction techniques and may be refined using computer software such as X-PLOR (Yale University, 1992, distributed by Molecular Simulations, Inc.; see e.g. Meth Enzymol (1985) volumes 114 & 115, eds Wyckoff H W et al.,; U.S. 2004/0014194), and BUSTER (Bricogne G (1993) Acta Crystallogr D Biol Crystallogr 49 (Pt 1): 37-60; Bricogne G (1997) Meth Enzymol 276A: 361-423, ed Carter C W; Roversi P et al., (2000) Acta Crystallogr D Biol Crystallogr 56 (Pt 10): 1316-1323). Mutagenesis mapping studies may be accomplished using any method known to one of skill in the art. See, e.g., Champe M et al., (1995) J Biol Chem 270:1388-1394 and Cunningham BC & Wells JA (1989) Science 244:1081-1085 for a description of mutagenesis techniques, including alanine scanning mutagenesis techniques.

As used herein, the term “extracellular domain” (or “ECD”) may refer to a portion of a polypeptide that, when the polypeptide is present in a cell membrane, is understood to reside outside of the cell membrane, in the extracellular space. Ecto domain may be used herein interchangeably with extracellular domain.

As used herein, the term “extracellular ligand-binding domain” may refer to an oligo- or polypeptide that is capable of binding a ligand, e.g., a cell surface molecule. For example, an extracellular ligand-binding domain may be selected to recognize a ligand that acts as a cell surface marker on target cells associated with a particular disease state (e.g., cancer). Non-limiting examples of cell surface markers that may act as ligands include those associated with viral, bacterial and parasitic infections, autoimmune disease and cancer cells.

As used herein, the term “gene” for the purposes of the present disclosure, includes a DNA region encoding a gene product, as well as all DNA regions which regulate the production of the gene product, whether or not such regulatory sequences are adjacent to coding and/or transcribed sequences. Accordingly, in various embodiments, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

As used herein the term “fusion polypeptide” or “fusion protein” are used interchangeably and may refer to a polypeptide comprising at least two segments. Generally, a polypeptide containing at least two such segments is considered to be a fusion polypeptide if the two segments are moieties that (1) are not comprised in nature in the same peptide, and/or (2) have not previously been linked or connected to one another in a single polypeptide, and/or (3) have been linked or connected to one another through action of the hand of man. In embodiments, a CAR is a fusion protein. In various embodiments, a T cell receptor may include a fusion protein.

As used herein, the term “immunotherapy” may refer to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy may include adoptive T cell therapy, tumor-infiltrating lymphocyte (TIL) immunotherapy, autologous cell therapy, engineered autologous cell therapy (eACT™), and allogeneic T cell transplantation. However, one of skill in the art would recognize that the conditioning methods disclosed herein would enhance the effectiveness of any transplanted T cell therapy. Examples of T cell therapies are described in U.S. Patent Publication Nos. 2014/0154228 and 2002/0006409, U.S. Pat. Nos. 7,741,465, 6,319,494, 5,728,388, and International Publication No. WO 2008/081035. In some embodiments, the immunotherapy comprises CAR T cell treatment. In various embodiments, the methods for upregulating low-density lipoprotein receptor (LDL-R) expression described herein may be incorporated into any of the immunotherapy workflows described herein and elsewhere.

T cells of the immunotherapy may come from any source known in the art. For example, T cells may be differentiated in vitro from a hematopoietic stem cell population, or T cells may be obtained from a subject. T cells may be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by reference in its entirety.

As used herein, the terms “intracellular signaling domain” and “signaling domain” are used interchangeably and may refer to the part of the chimeric antigen receptor protein that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. As used herein, the term “effector function” may refer to a specialized function of the cell. Effector function of the T cell, for example, may be cytolytic activity or help or activity including the secretion of a cytokine. Thus, the terms “intracellular signaling domain” or “signaling domain,” used interchangeably herein, refer to the portion of a protein which transduces the effector function signal and that directs the cell to perform a specialized function. While usually the entire intracellular signaling domain may be employed, in many cases it is not necessary to use the entire domain. To the extent that a truncated portion of an intracellular signaling domain is used, such truncated portion may be used in place of the entire domain as long as it transduces the effector function signal. The term intracellular signaling domain may include any truncated portion of the intracellular signaling domain sufficient to transducing effector function signal. The intracellular signaling domain is also known as the, “signal transduction domain,” and is typically derived from portions of the human CD3 or FcRy chains.

It is known that signals generated through the T cell receptor alone are insufficient for full activation of the T cell and that a secondary, or costimulatory signal is also required. Thus, T cell activation can be said to be mediated by two distinct classes of cytoplasmic signaling sequences: those that initiate antigen dependent primary activation through the T cell receptor (primary cytoplasmic signaling sequences) and those that act in an antigen independent manner to provide a secondary or costimulatory signal (secondary cytoplasmic signaling sequences). Cytoplasmic signaling sequences that act in a costimulatory manner may contain signaling domains which are known as immunoreceptor tyrosine-based activation domain or ITAMs.

Non-limiting examples of ITAM containing primary cytoplasmic signaling sequences that may be of particular use in the embodiments described herein may include those derived from DAP10, DAP12, TCRzeta, FcRgamma, FcRbeta, CD3zeta, CD3gamma, CD3delta, CD3epsilon, CD5, CD22, CD79a, CD79b and CD66d.

The term “lymphocyte” as used herein includes natural killer (NK) cells, T cells, or B cells. NK cells are a type of cytotoxic (cell toxic) lymphocyte that represent a major component of the inherent immune system. NK cells reject tumors and cells infected by viruses. It works through the process of apoptosis or programmed cell death. They were termed “natural killers” because they do not require activation in order to kill cells. T cells play a major role in cell-mediated-immunity (no antibody involvement). Its T cell receptors (TCR) differentiate themselves from other lymphocyte types. The thymus, a specialized organ of the immune system, is primarily responsible for the T cell's maturation. There are six types of T cells, namely: Helper T cells (e.g., CD4+ cells), Cytotoxic T cells (also known as TC, cytotoxic T lymphocyte, CTL, T-killer cell, cytolytic T cell, CD8+ T cells or killer T cell), Memory T cells ((i) stem memory TSCM cells, like naive cells, are CD45RO−, CCR7+, CD45RA+, CD62L+ (L-selectin), CD27+, CD28+ and IL-7Ra+, but they also express large amounts of CD95, IL-2RB, CXCR3, and LFA-1, and show numerous functional attributes distinctive of memory cells); (ii) central memory TCM cells express L-selectin and the CCR7, they secrete IL-2, but not IFNγ or IL-4, and (iii) effector memory TEM cells, however, do not express L-selectin or CCR7 but produce effector cytokines like IFNγ and IL-4), Regulatory T cells (Tregs, suppressor T cells, or CD4+CD25+ regulatory T cells), Natural Killer T cells (NKT) and Gamma Delta T cells. B-cells, on the other hand, play a principal role in humoral immunity (with antibody involvement). It makes antibodies and antigens and performs the role of antigen-presenting cells (APCs) and turns into memory B-cells after activation by antigen interaction. In mammals, immature B-cells are formed in the bone marrow, where its name is derived from.

As used herein, the term “nucleic acid” may refer to any polymeric chain of nucleotides. A nucleic acid may be DNA, RNA, or a combination thereof. In various embodiments, a nucleic acid may comprise one or more natural nucleic acid residues. In various embodiments, a nucleic acid may comprise of one or more nucleic acid analogs. In various embodiments, nucleic acids may be prepared by one or more of isolation from a natural source, enzymatic synthesis by polymerization based on a complementary template (in vivo or in vitro), reproduction in a recombinant cell or system, and chemical synthesis. In various embodiments, a nucleic acid may be at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more bases long (e.g., 20 to 100, 20 to 500, 20 to 1000, 20 to 2000, or 20 to 5000 or more bases). In various embodiments, a nucleic acid is partly or wholly single stranded. In various embodiments, a nucleic acid may be partly or wholly double stranded. In various embodiments a nucleic acid may have a nucleotide sequence comprising at least one element that encodes, or is the complement of a sequence that encodes, a polypeptide.

As used herein, the terms “patient” and “subject” are used interchangeably herein and may mean any living thing (e.g., a human or organism having eukaryotic cells) who is being treated for an abnormal physiological condition, such as cancer or has been formally diagnosed with a disorder, those without formally recognized disorders, those receiving medical attention, those at risk of developing the disorders, etc. The terms “subject” and “patient” are used interchangeably herein and include both human and non-human animal subjects.

As used herein, the terms “peptide,” “polypeptide,” and “protein” are used interchangeably and may refer to a compound comprised of amino acid residues covalently linked by peptide bonds. A protein or peptide contains at least two amino acids, and no limitation is placed on the maximum number of amino acids that can comprise a protein's or peptide's sequence. Polypeptides may include any peptide or protein comprising two or more amino acids joined to each other by peptide bonds. As used herein, the terms “peptide,” “polypeptide,” and “protein” refer to both short chains, which also commonly are referred to in the art as peptides, oligopeptides and oligomers, for example, and to longer chains, which generally are referred to in the art as proteins, of which there are many types. “Polypeptides” may include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “reference” may describe a standard or control relative to which a comparison is performed. For example, in various embodiments, an agent, animal, individual, population, sample, sequence, or value of interest is compared with a reference or control that is an agent, animal, individual, population, sample, sequence, or value. In various embodiments, a reference or control may be tested, measured, and/or determined substantially simultaneously with the testing, measuring, or determination of interest. In various embodiments, a reference or control may include a historical reference or control, optionally embodied in a tangible medium. Generally, a reference or control may be determined or characterized under comparable conditions or circumstances to those under assessment. When sufficient similarities are present to justify reliance on and/or comparison to a selected reference or control.

As used herein, the term “RNA binding molecule” is a molecule that can bind to RNA. Such RNA binding molecule can be a polypeptide, a domain of a protein, a domain within a larger protein or a polynucleotide. In some embodiments, the polynucleotide is DNA, while in other embodiments, the polynucleotide is RNA. In some embodiments, the RNA binding molecule is a small interfering RNA, such as a siRNA or miRNA, for example an siRNA or miRNA specific for Bach2, TCF7, Blimp-1, or A20.

As used herein, the term “sample” may generally refers to an aliquot of material obtained or derived from a source of interest. In various embodiments, a sample or a source of interest may include an apheresis material. In various embodiments, a source of interest is a biological or environmental source. In various embodiments, a source of interest may comprise a cell or an organism, such as a cell population, tissue, or animal (e.g., a human). In various embodiments, a source of interest may include biological tissue or fluid. In various embodiments, a biological tissue or fluid may comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoncal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In various embodiments, a biological fluid may comprise an intracellular fluid, an extracellular fluid, an intravascular fluid (blood plasma), an interstitial fluid, a lymphatic fluid, and/or a transcellular fluid. In various embodiments, a biological fluid may comprise a plant exudate. In various embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brochcoalvealar, ductal, nasal, ocular, oral, uterine, vaginal, or other washing or lavage). In various embodiments, a biological sample may comprise cells obtained from an individual. In various embodiments, a sample may include a “primary sample” obtained directly from a source of interest by any appropriate means. In various embodiments, as will be clear from context, the term “sample” may refer to a preparation that is obtained by processing (e.g., by removing one or more components of and/or by adding one or more agents to) a primary sample. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

As used herein, the term “sequence” may refer to a nucleotide sequence of any length, which may be DNA or RNA, may be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

As used herein, the term “single chain variable fragment,” “single-chain antibody variable fragments,” or “scFv” antibodies refer to forms of antibodies comprising the variable regions of only the heavy and light chains, connected by a linker peptide.

As used herein, the term “stimulation,” may refer to a primary response induced by binding of a stimulatory molecule with its cognate ligand, wherein the binding mediates a signal transduction event. A “stimulatory molecule” may include a molecule on a T cell, e.g., the T cell receptor (TCR)/CD3 complex, that specifically binds with a cognate stimulatory ligand present on an antigen present cell. A “stimulatory ligand” may include a ligand that when present on an antigen presenting cell (e.g., an APC, a dendritic cell, a B-cell, and the like) can specifically bind with a stimulatory molecule on a T cell, thereby mediating a primary response by the T cell, including, but not limited to, activation, initiation of an immune response, proliferation, and the like. Stimulatory ligands include, but are not limited to, an anti-CD3 antibody (such as OKT3), an MHC Class I molecule loaded with a peptide, a superagonist anti-CD2 antibody, and a superagonist anti-CD28 antibody.

As used herein, the terms “T cell receptor” and “TCR” are used interchangeably and may refer to antigen-recognition molecules present on the surface of T cells. During normal T cell development, each of the four TCR genes, α, β, γ, and δ, may rearrange leading to highly diverse TCR proteins. Examples of TCR based T cell therapies are disclosed in International Patent Application Nos. PCT/US2013/059608 and PCT/US2015/033129, which are hereby incorporated herein by reference in their entirety.

As used herein, the term “therapeutic agent” may refer to any agent that elicits a desired pharmacological effect when administered to an organism. In some embodiments, an agent is considered to be a therapeutic agent if it demonstrates a statistically significant effect across an appropriate population. In various embodiments, the appropriate population may be a population of model organisms or human subjects. In various embodiments, an appropriate population may be defined by various criteria, such as a certain age group, gender, genetic background, preexisting clinical conditions, in accordance with presence or absence of a biomarker, etc. In various embodiments, a therapeutic agent may be a substance that may be used to alleviate, ameliorate, relieve, inhibit, prevent, delay onset of, reduce severity of, and/or reduce incidence of one or more symptoms or features of a disease, disorder, and/or condition. In various embodiments, a therapeutic agent may include an agent that has been or is required to be approved by a government agency before it may be marketed for administration to humans. In various embodiments, a therapeutic agent may be an agent for which a medical prescription is required for administration to humans. In various embodiments, a therapeutic agent may include a CAR T cell. In various embodiments, a therapeutic agent may include a CD38 therapeutic agent.

As used herein, the term “transmembrane domain” may refer to a domain of a polypeptide that includes at least one contiguous amino acid sequence that traverses a lipid bilayer when present in the corresponding endogenous polypeptide when expressed in a mammalian cell. For example, a transmembrane domain can include one, two, three, four, five, six, seven, eight, nine, or ten contiguous amino acid sequences that each traverse a lipid bilayer when present in the corresponding endogenous polypeptide when expressed in a mammalian cell. In various embodiments, a transmembrane domain may include at least one (e.g., two, three, four, five, six, seven, eight, nine, or ten) contiguous amino acid sequence (that traverses a lipid bilayer when present in the corresponding endogenous polypeptide when expressed in a mammalian cell) that has α-helical secondary structure in the lipid bilayer. In various embodiments, a transmembrane domain may include two or more contiguous amino acid sequences (that each traverse a lipid bilayer when present in the corresponding endogenous polypeptide when expressed in a mammalian cell) that form α-barrel secondary structure in the lipid bilayer. In various embodiments, the transmembrane region or domain may include the portion of a CAR that anchors the extracellular binding portion to the plasma membrane of the immune effector cell and facilitates binding of the binding domain to the target antigen. The transmembrane domain may include a CD3zeta transmembrane domain, however other transmembrane domains that may be employed include those obtained from CD8alpha, CD4, CD28, CD45, CD9, CD16, CD22, CD33, CD64, CD80, CD86, CD134, CD137, NKG2D, 2B4 and CD154. In various embodiments, the transmembrane domain may be synthetic in which case it would comprise predominantly hydrophobic residues such as leucine and valine.

As used herein, the terms “treatment” and “treating” of a subject are used interchangeably and may refer to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In some embodiments, “treatment” or “treating” includes a partial remission. In another embodiment, “treatment” or “treating” includes a complete remission. In various embodiments, a treatment may comprise administering a CAR T cell therapy product to a patient. In various embodiments, a treatment may comprise administering antibodies to a patient such as a monoclonal or polyclonal antibody. The monoclonal antibody may include daratumumab or isatuximab.

As used herein, the term “vector” may refers to a recipient nucleic acid molecule modified to comprise or incorporate a provided nucleic acid sequence. One type of vector may be a “plasmid,” which may refer to a circular double stranded DNA molecule into which additional DNA may be ligated. Another type of vector may be a viral vector, wherein additional DNA segments may be ligated into the viral genome. Certain vectors may be capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome. Moreover, certain vectors comprise sequences that direct expression of inserted genes to which they are operatively linked. Such vectors may be referred to herein as “expression vectors.” Standard techniques may be used for engineering of vectors, e.g., as found in Sambrook et al., Molecular Cloning: A Laboratory Manual (2d ed., Cold Spring Harbor Laboratory Press, Cold Spring Harbor, N.Y. (1989)), which is incorporated herein by reference.

The disclosure further provides diagnostic, prognostic and therapeutic methods, which are based, at least in part, on determination of the expression level of a gene of interest identified herein.

### II. Overview

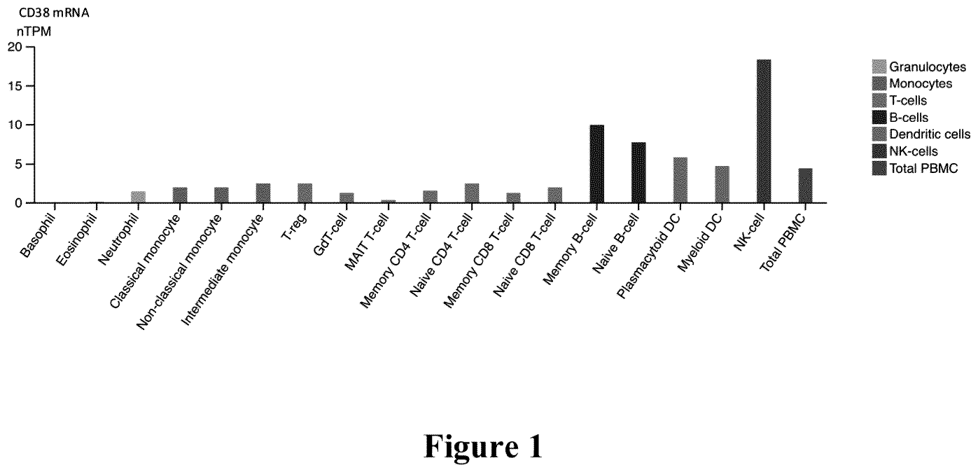

The synergistic effects of a combination therapy including T cells and a CD38 targeting compound that have not been available in a combination therapy format because of issues resulting from negative interactions between T cells and CD38 targeting compounds until the development of the technologies described herein. Such issues arise due to expression of CD38 on a variety of cell types. FIG. 1 illustrates a bar graph of summarized data showing CD38 mRNA expression levels in various cell types, e.g., basophils, cosinophils, classical monocytes, non-classical monocytes, T-regs, GdT-cells, MAIT T-cells, Memory CD4 T-cells, naïve CD4 T-cells, Memory CD8 T-cells, naïve CD8 T-cells, Memory B-cells, Naïve B-cells, Plasmacytoid dendritic cells, myeloid dendritic cells, natural killer (NK) cells, and peripheral blood mononuclear cells (PBMCs). Normalized data downloaded was from Human Protein Atlas (https://www.proteinatlas.org/). Additionally, and as discussed herein, multiple myeloma cells are at least one contemplated target for the combination therapy technology described herein due to increased CD38 expression.

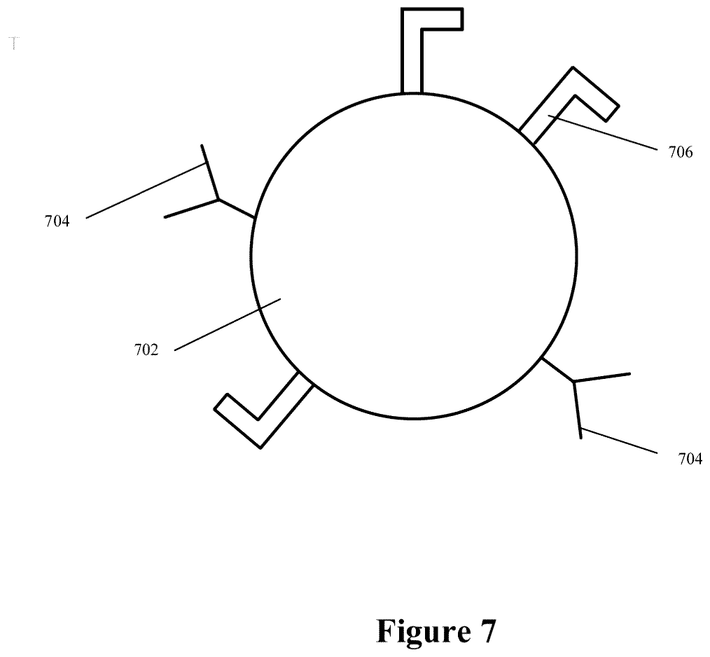

Under normal circumstances, T cells commonly express CD38 antigen. For example, without intervention cell therapy products are likely to include cells expressing CD38 (e.g., engineered T cells, activated T cells, etc.) and would be susceptible to targeting by CD38 targeting compounds (e.g., isatuximab, daratumumab, etc.). As such, NK cell targeting, and subsequent destruction of engineered T cells will likely reduce the effectiveness of co-therapies including both engineered T cell and CD38 targeting compounds. See, e.g., FIG. 7 depicting an embodiment where engineered T cells do not include a CD38 expression modification and are, therefore, susceptible to NK cell targeting and subsequent destruction (NK cells not shown).

The technologies described herein include engineered T cells that may be modified (e.g., genetically or otherwise) to evade binding by CD38 targeting compounds (e.g., non-limiting examples may include antibodies, a DARPin, and/or a receptor trap based on CD31 or any other natural ligand of CD38) and/or NK cells in accordance with various embodiments. Non-limiting examples of the way in which the expression modification of the T cell may be accomplished includes gene knockout by Cas9, Cas12, TALEN, Zinc Finger, base editor, and/or any other gene editing system, or gene knockdown by Cas13, RNA interference, CRISPRi, and/or any epigenetic editing system, disruption of the CD38 coding region by knocking a genetic element into the CD38 locus, and/or by introducing mutations into CD38 that alter anti-CD38 binding epitopes by base editing, prime editing, or homology directed repair in accordance with various embodiments. The engineered T cells may be engineered to express a recombinant TCR or CAR. The T cells may be engineered to express additional transgenes, and additional gene edits can be made to facilitate immune evasion or enhance T cell fitness. See Section IV. Examples.

The technologies described herein may be applicable to any circumstances involving specific targeting and/or destruction of a cell. More specifically, the technologies described herein may be applicable to cancer treatment. Due to the observation that lymphoma and multiple myeloma cells overexpress CD38 molecules, the embodiments described herein including use of engineered T cells having a CD38 expression modification combined with use of a CD38 targeting compound as a treatment may be particularly effective.

i) CAR T cell Protection from Natural Killer Cell-Mediated Rejection

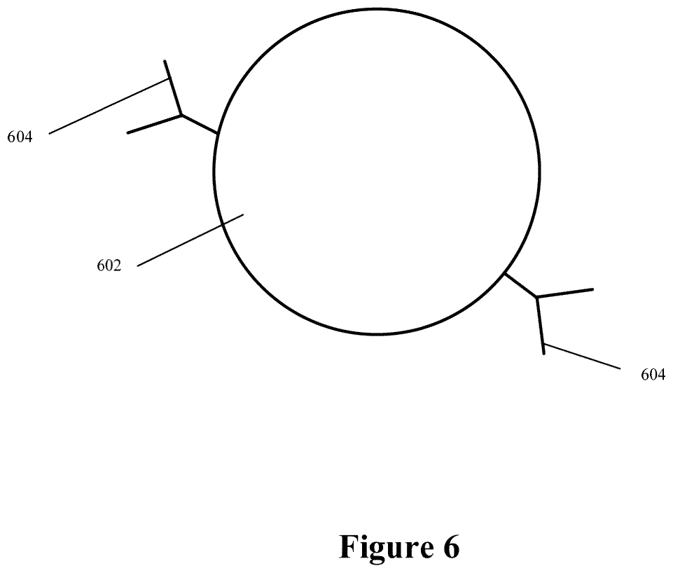

Unmodified T cells or CAR T cells express CD38 along with many other cell types (see, e.g., FIG. 1). In the presence of an anti-CD38 antibody, Natural killer (NK) cells target cells expressing CD38 antigen which makes unmodified T cells and/or CAR T cells expressing CD38 antigen susceptible to NK cell mediated recognition and destruction. As the skilled artisan will appreciate, under most conditions the patient benefits more when they are administered a CAR T cell therapy product that is not susceptible to being degraded or compromised by NK mediate activity (e.g., see FIGS. 6-11). As discussed herein, T cells and/or CAR T cells may be modified such that their expression of CD38 is changed, reduced, increased, eliminated, or altered in some way in accordance with the embodiments described herein. Additionally, reduction in the total number of NK cells present may provide additional protections. A variety of embodiments are described herein to achieve NK cell depletion which may include administration of a CD38 targeting compound targeting CD38 antigen present on NK cells and/or use of a lymphodepletion protocol in accordance with various embodiments.

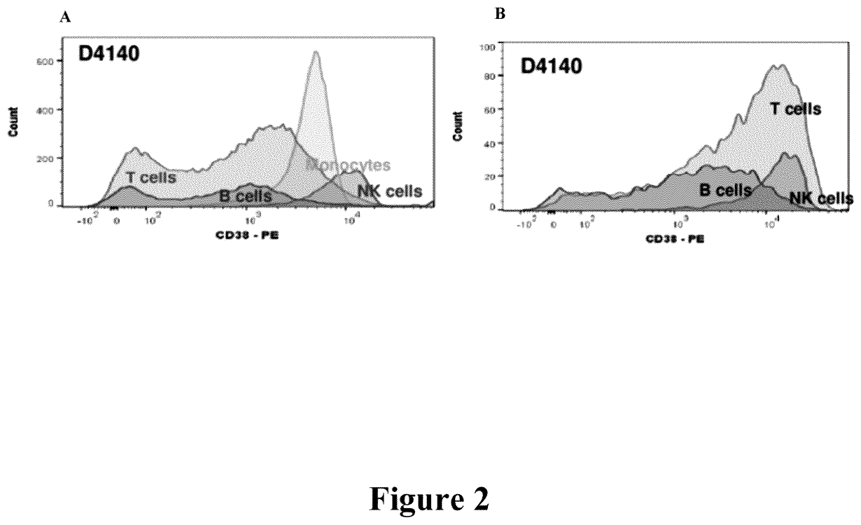

The presently described embodiments may mitigate and/or resolve the issue of T cell and/or CAR T cell rejection mediated through Natural Killer (NK) cell activity. FIG. 2 is experimental data showing and increase in CD38 expression on T cells after undergoing a stimulation procedure (e.g., activation). Activation is a common manufacturing step for many engineered T cells. As such, engineered T cells are often even more susceptible to CD38 targeting than normal T cells. FIG. 2, panel A shows high levels of CD38 expression on T cells and natural killer cells (NK cells). FIG. 2, panel B shows an increase in CD38 expression levels on T cells and NK cells after undergoing a stimulation procedure compared to before undergoing the procedure. T cell activation is also expected to occur in patients when CAR-T cells engage with antigen-positive target cells.

One of the mechanisms through which NK cells operate is antibody-mediated recognition of CD38 antigens and destruction of their associated cell (e.g., a CAR T cell infused into a patient undergoing cancer treatment). In various embodiments, one or more of the CD38 targeting compounds described herein and elsewhere may be used to depleted patient NK cells. A non-limiting example of a benefit may include diminished rejection of allogeneic CAR T cells.

### ii) Compatibility and Synergistic Anti-Tumor Activity

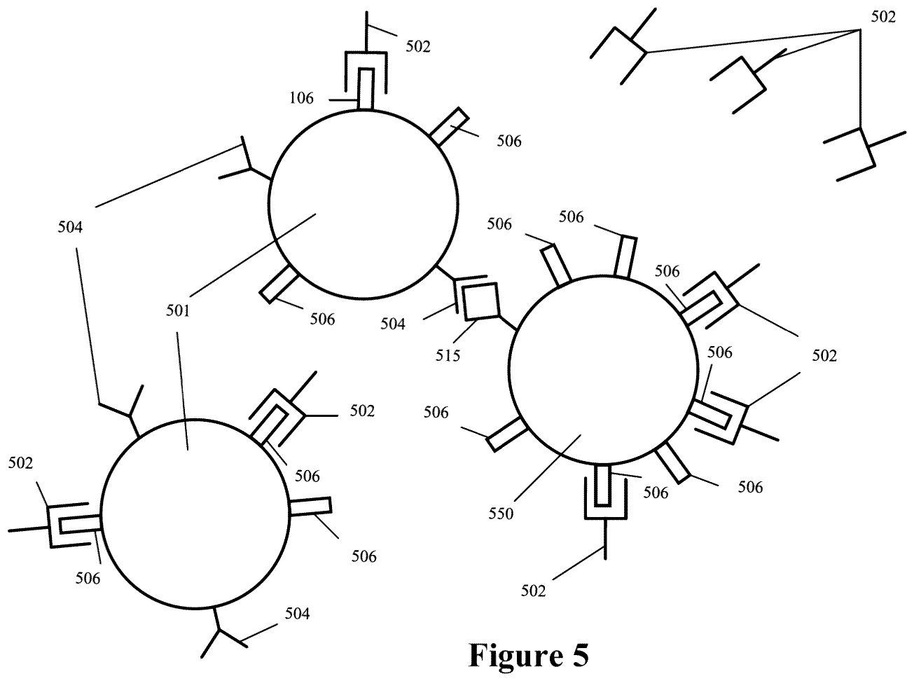

In various embodiments, both autologous and allogeneic therapies using engineered T cells may suffer from incompatibility issues which may be resolved using the technology described herein. In various embodiments, there may be a benefit to co-treatment using a CD38 targeting compound and an engineered T cell not including a CD38 expression modification. However, the CD38+ engineered T cell shown in FIG. 5 may be susceptible to various antibody-dependent killing mechanisms or CD38 targeting compounds which may lead to decreased effectiveness. Various other embodiments described herein overcome this deficiency by introduction and of a CD38 expression modification into an engineered T cell.

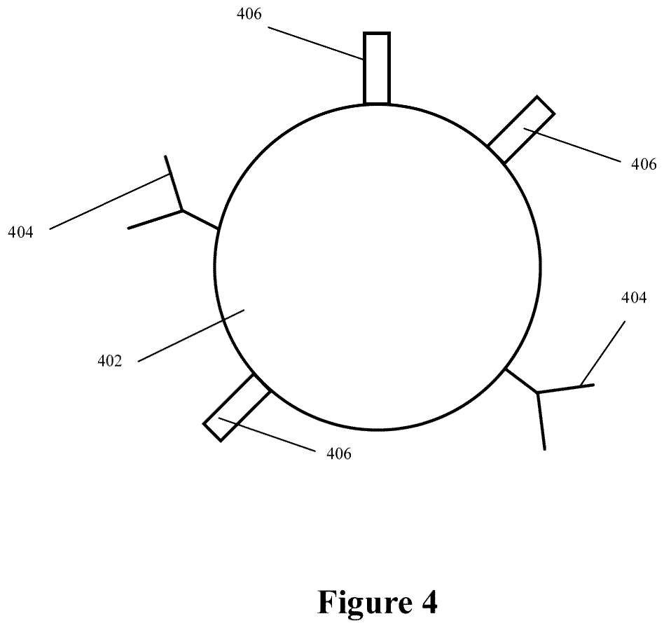

FIG. 4 is an illustration of an engineered T cell 402 expressing a chimeric antigen receptor (CAR) 404 and a CD38 antigen 406 that is lacking a CD38 expression modification in accordance with various embodiments. Naturally occurring T cells may resemble the illustration shown in FIG. 4. The engineered T cells described herein may target any of various ligands described throughout this disclosure.

FIG. 5 is an illustration of a system in which a CD38 targeting compound 502 is targeting CD38 antigens 506 on both engineered T cells 501 and a cancer cell or target cell 550. In various embodiments, the engineered T cells 501 may target include CD20, CD19, CLL-1, BCMA, EGFR, HER2, GPC3, GD2, IL13RA, or any combination thereof. In various embodiments, the engineered T cells 501 may comprise an antigen binding molecule including a binding motif that binds an antigen selected from the group comprising 5T4, alphafetoprotein, B cell maturation antigen (BCMA), B cell receptor, CA-125, carcinoembryonic antigen, CD19, CD20, CD22, CD23, CD30, CD33, CD40, CD56, CD79, CD78, CD123, CD138, c-Met, CSPG4, IgM, C-type lectin-like molecule 1 (CLL-1), EGFRvIII, epithelial tumor antigen, ERBB2, FLT3, folate binding protein, GD2, GD3, HER1-HER2 in combination, HER2-HER3 in combination, HER2/Neu, HERV-K, HIV-1 envelope glycoprotein gp41, HIV-1 envelope glycoprotein gpl20, IL-IIRalpha, kappa chain, lambda chain, melanoma-associated antigen, mesothelin, MUC-1, mutated p53, mutated ras, prostate-specific antigen, ROR1, VEGFR2, EphA3 (EPH receptor A3), BAFFR (B-cell activating factor receptor), and combinations thereof.

In various embodiments, the T cells 501 may express CD38 antigen 506 and a CAR 504. As depicted in FIG. 5, the T cells 501 may still express a CD38 antigen 506 in accordance with some embodiments. In such embodiments, a CD38 targeting compound 502 may bind to the CD38 antigen 506 molecule expressed on the cell surface of the engineered T cells 501. Binding 515 can be seen between an engineered T cell 501 and a cancer cell 550.

In this embodiment, issues arise because CD38 targeting compound 502 cannot differentiate (or has difficulty differentiating) between the engineered T cells 501 and the cancer cell 550. In FIG. 5, it can be seen that although the engineered T cells 501 and the CD38 targeting compound 502 work in conjunction to target and destroy the cancer cell 550, the CD38 targeting compound 502 also binds to and destroys the engineered T cells 501 which reduces an effectiveness of combination therapies including both engineered T cells 501 and CD38 targeting compounds 502. The embodiments (e.g., embodiments depicted in FIGS. 6-11) described herein help to resolve these compatibility issues and increases synergistic capabilities.

### III. Exemplary Cells for Combination Therapy

An obstacle for any combination therapy may include interactions between one or more therapeutics. For example, some therapeutic compounds come with a warning to not combine them with certain other therapeutic compounds due to adverse patient reactions which could lead to safety concerns. Another possible outcome may be that one or more of the therapeutic compounds of the combination therapy may be inhibited or neutralized by one or more of the other therapeutic compounds of the combination therapy leading to worse patient outcomes. In various embodiments, the engineered T cells described herein have been optimized to reduce these kinds of negative interactions between therapeutic compounds used in a combination therapy product. A unique issue involves engineered T cells used in combination therapy relates to their inherent nature as a living cell. Living cells may be suspectable to being targeted by the other one or more therapeutics used in a combination therapy. More specifically, native T cells typically express a CD38 antigen which may be subject to being targeted by other therapeutics in the combination therapy specific for CD38. As such, the engineered T cells herein may use various methods and techniques to evade CD38 targeting in accordance with various embodiments.

### i) Exemplary Engineered T Cells Expressing a Chimeric Antigen Receptor (CAR) Including a CD38 Expression Modification Resulting in a Change in Cell Surface CD38 Antigen Expression Levels

FIG. 6 is an illustration of an engineered T cell 602 expressing chimeric antigen receptors (CARs) 604 without expression of CD38 antigens in accordance with various embodiments. In various embodiments, expression may be reduced instead of eliminated entirely. For example, there are a variety of techniques described herein and elsewhere capable of modulating CD38 antigen expression levels. In various embodiments, the engineered T cell 602 may target include CD20, CD19, CLL-1, BCMA, EGFR, HER2, GPC3, GD2, IL13RA, or any combination thereof. In various embodiments, the engineered T cell 602 may comprise an antigen binding molecule including a binding motif that binds an antigen selected from the group comprising 5T4, alphafetoprotein, B cell maturation antigen (BCMA), B cell receptor, CA-125, carcinoembryonic antigen, CD19, CD20, CD22, CD23, CD30, CD33, CD40, CD56, CD79, CD78, CD123, CD138, c-Met, CSPG4, IgM, C-type lectin-like molecule 1 (CLL-1), EGFRvIII, epithelial tumor antigen, ERBB2, FLT3, folate binding protein, GD2, GD3, HER1-HER2 in combination, HER2-HER3 in combination, HER2/Neu, HERV-K, HIV-1 envelope glycoprotein gp41, HIV-1 envelope glycoprotein gp120, IL-IIRalpha, kappa chain, lambda chain, melanoma-associated antigen, mesothelin, MUC-1, mutated p53, mutated ras, prostate-specific antigen, ROR1, VEGFR2, EphA3 (EPH receptor A3), BAFFR (B-cell activating factor receptor), and combinations thereof.

FIG. 7 is an illustration of an engineered T cell 702 expressing chimeric antigen receptors (CARs) 704 and CD38 antigens 706 wherein the CD38 antigens 706 have been modified in accordance with various embodiments. In various embodiments, the engineered T cell 702 may target include CD20, CD19, CLL-1, BCMA, EGFR, HER2, GPC3, GD2, or any combination thereof. In various embodiments, the engineered T cell 702 may comprise an antigen binding molecule including a binding motif that binds an antigen selected from the group comprising 5T4, alphafetoprotein, B cell maturation antigen (BCMA), B cell receptor, CA-125, carcinoembryonic antigen, CD19, CD20, CD22, CD23, CD30, CD33, CD40, CD56, CD79, CD78, CD123, CD138, c-Met, CSPG4, IgM, C-type lectin-like molecule 1 (CLL-1), EGFRvIII, epithelial tumor antigen, ERBB2, FLT3, folate binding protein, GD2, GD3, HER 1-HER2 in combination, HER2-HER3 in combination, HER2/Neu, HERV-K, HIV-1 envelope glycoprotein gp41, HIV-1 envelope glycoprotein gpl20, IL-IIRalpha, kappa chain, lambda chain, melanoma-associated antigen, mesothelin, MUC-1, mutated p53, mutated ras, prostate-specific antigen, ROR1, VEGFR2, EphA3 (EPH receptor A3), BAFFR (B-cell activating factor receptor), and combinations thereof.

Various techniques are described herein and elsewhere for achieving an optimized amount of CD38 expression of an engineered T cell. For example, in various embodiments, a CD38 gene knock-out may be used. In various embodiments, the gene knock-out may be Cas9 mediated. In various embodiments, the Cas9 mediated gene knock-out may use a complex including a Cas9 molecule and a guide nucleotide sequence.

In various embodiments, the nucleotide guide sequence may include SEQ ID NO: 1. In various embodiments the nucleotide guide sequence may include SEQ ID NO: 2. In various embodiments the nucleotide guide sequence may include SEQ ID NO: 3. In various embodiments, the nucleotide guide sequence may include SEQ ID NO: 4.

In various embodiments, the gene knock-out may be Cas12 mediated.

In various embodiments, the gene knock-out may be base editor mediated. In various embodiments, the base editor may include a cytosine base editor (CBE). In various embodiments, the cytosine base editor may include a cytidine base editor comprised of a Cas9 nickase fused to a cytidine deaminase derived from APOBEC1. In various embodiments, the base editor may include an adenine base editor (ABE). In various embodiments, the cytidine base editor may be comprised of Cas9 nickase fused to cytidine deaminase derived from APOBEC1. In various embodiments, the adenine base editor may be comprised of Cas9 nickase fused to deoxyadenosine deaminase derived TadA.

In various embodiments, the base editor may be complexed with a nucleotide guide sequence. In various embodiments, the nucleotide guide sequence may include SEQ ID NO: 5. In various embodiments, the nucleotide guide sequence may include SEQ ID NO: 6. In various embodiments, the nucleotide guide sequence may include SEQ ID NO: 7. In various embodiments, the nucleotide guide sequence may include SEQ ID NO: 8. In various embodiments, the nucleotide guide sequence may include SEQ ID NO: 9. In various embodiments, the nucleotide guide sequence may include SEQ ID NO: 10.

In various embodiments, the CD38 expression modification may use epigenetic editing. In various embodiments, the epigenetic editing may use a TALEN. In various embodiments, the epigenetic editing may use a zinc finger nuclease. In various embodiments, the epigenetic editing may use CRISPRi.

In various embodiments, the CD38 expression modification may comprise a single amino acid substitution. In various embodiments, the single amino acid substitution occurs at an epitope specific for Daratumumab. In various embodiments, the single amino acid substitution occurs at an epitope specific for Isatuximab.

In various embodiments, the single amino acid substitution is mediated by a base editor. In various embodiments, the substitution replaces serine 274 of SEQ ID NO: 23. In various embodiments, the substitution replaces the serine with a phenylalanine. In various embodiments, the base editor includes a nucleotide guide sequence comprising SEQ ID NO: 11.

In various embodiments, the substitution may replace threonine 116. In various embodiments, the substitution may replace the threonine with an alanine. In various embodiments, the base editor may include a nucleotide guide sequence comprising SEQ ID NO: 17.

In various embodiments, the base editor may include a nucleotide guide sequence comprising SEQ ID NO: 16. In various embodiments, the base editor may include a nucleotide guide sequence comprising SEQ ID NO: 18. In various embodiments, the base editor may include a nucleotide guide sequence comprising SEQ ID NO: 19.

In various embodiments, the engineered T cell may express a CD38 antigen nucleotide encoding sequence comprising SEQ ID NO: 21. In various embodiments, the engineered T cell may express a CD38 antigen protein molecule comprising SEQ ID NO: 23. In various embodiments, the engineered T cell may express a CD38 antigen protein molecule comprising SEQ ID NO: 24.

In various embodiments, a cell or a population of cells may be used as part of a combination therapy treatment for a patient. For example, engineered T cells may be used in accordance with various embodiments. Additionally, engineered T cells may include CAR T cells which may be used according to various embodiments.

In various embodiments, an engineered T cell may include a CD38 expression modification. A non-limiting example of a technique used to modify a T cell may include molecular tools (e.g., genome editing nucleases). “Genome editing nucleases include different categories of enzymes: meganucleases (MNs), zinc finger nucleases (ZFNs), clustered regularly interspaced short palindromic repeats (CRISPR)-CRISPR associated protein (Cas) and transcription activator-like effector nuclease (TALENs). Transposable elements are also a category of molecular tools which includes different members, for example Sleeping Beauty (SB), PiggyBac (PB), Tol2 and TcBuster. Transposons have been used for genetic studies and can serve as gene delivery tools. Molecular tools to rewire host's gene expression also include episomes, which are divided into different categories depending on their molecular structure. Finally, RNA interference is commonly used to regulate gene expression through the administration of small interfering RNA (siRNA), short hairpin RNA (shRNA) and bi-functional shRNA molecules.” Tsai, HC., Pietrobon, V., Peng, M. et al. Current strategies employed in the manipulation of gene expression for clinical purposes. J Transl Med 20, 535 (2022). https://doi.org/10.1186/s12967-022-03747-3 the disclosure of which is hereby incorporated by reference in its entirety for all purposes.

In various embodiments, the CD38 expression modification may be created using a gene knock-out approach. In various embodiments, a gene knock-out may include gene deletion. In various embodiments, a gene knock-out may include gene inactivation. Non-limiting methods of gene knock-out methods may use targeted homologous recombination, CRISPR-Cas9, CRISPR-Cas 12, and/or TALENs.

In various embodiments, targeted homologous recombination may be used to develop chimeric proteins (e.g., CARs). In various embodiments, homologous recombination may act by exchanging fragments of two parental proteins.

In various embodiments, an engineered T cell may be modified using a DNA binding molecule. In various embodiments, a modification may include a gene knock-out. In various embodiments, a modification may result in a protein modification (e.g., a modification to a CD38 antigen at an epitope which disrupts binding to a CD38 therapeutic). In various embodiments, the modification and/or knock-out may be Cas9 mediated. In various embodiments, the modification and/or knock-out may be Cas 12 mediated.

Base editors “can directly convert target base pairs into another base pairs in DNA and RNA efficiently without inducing a DNA doublestrand break (DSB) or requiring donor DNA templates in living cells.” Liang Y, Chen F, Wang K, Lai L. Base editors: development and applications in biomedicine. Front Med. 2023 June; 17 (3): 359-387. doi: 10.1007/s11684-023-1013-y. Epub 2023 Jul. 12. PMID: 37434066. These and other base editors may be used to modify the T cell described herein in accordance with various embodiments.

In various embodiments, a modification may be accomplished using a base editor. For example, a base editor may be used to modify an encoding sequence to a CD38 antigen where the resulting modification leads to a protein modification to the epitope of the CD38 antigen. A base editor may be used to knock-out gene expression of CD38. In various embodiments, the base editor may include a cytidine base editor comprised of a Cas9 nickase fused to a cytidine deaminase derived from APOBEC1. In various embodiments, the base editor may include an adenine base editor comprised of a Cas9 nickase fused to a deoxyadenosine deaminase derived TadA.

A gene knock-in may refer to a genetic engineering method that involves the one-for-one substitution of DNA sequence information in a genetic locus or the insertion of sequence information not found within the locus. In various embodiments, the CD38 expression modification may comprise a CD38 gene knock-in. In various embodiments, the CD38 gene knock-in uses homology directed repair.

In various embodiments, the CD38 expression modification may use an RNA binding molecule. In various embodiments, the RNA binding molecule may include RNAi. In various embodiments, the RNA binding molecule may include siRNA. In various embodiments, the RNA binding molecule may include miRNA.

Various combination therapy techniques described herein may incorporate or use antisense therapy. In various embodiments, antisense oligonucleotides (ASOs) may target messenger RNA (mRNA). In various embodiments, the mRNA may encode a CD38 antigen. As used herein, ASOs may alter mRNA expression through a variety of mechanisms, including but not limited to ribonuclease H mediated decay of the pre-mRNA, direct steric blockage, and exon content modulation through splicing site binding on pre-mRNA. In various embodiments, the CD38 expression modification may use one or more anti-sense oligonucleotides (ASOs).

The CD38 expression modifications described herein may be carried out using epigenetic editing techniques in accordance with various embodiments. The terms genome editing and epigenetic editing may be used interchangeably herein. The epigenetic editing techniques described herein may be used to insert, delete, modify, or replace nucleotide sequences in a host genome (e.g., a T cell genome).

In various embodiments, the CD38 expression modification may be created or use a transcription activator-like effector-based nuclease (TALEN). In various embodiments, TALENs are restriction enzymes that may be engineered to cut specific sequences of DNA. In various embodiments a TALEN may include a TAL effector DNA-binding domain fused to a DNA cleavage domain. Transcription activator-like effectors (TALEs) may be engineered to bind to practically any desired DNA sequence (e.g., to precise locations of the CD38 gene) and then specifically cut at a desired cutting location. In various embodiments, TALEN constructs may be inserted into plasmids and then the target cells (e.g., a T cell) may be transfected with the plasmids and the gene products may expressed and enter the nucleus to specifically target a location of a genome. In other embodiments, TALEN constructs may be delivered to the cells as mRNAs. This delivery method reduces likelihood or removes the possibility of genomic integration of the TALEN system protein. In various embodiments, an mRNA vector may also increase the level of homology directed repair (HDR) and the success of introgression during gene editing.

In various embodiments, the CD38 expression modification may be created by or use a zinc finger nuclease. In various embodiments zinc-finger nucleases (ZFNs) may be artificial restriction enzymes comprising a fused zinc finger DNA-binding domain to a DNA-cleavage domain. As used herein zinc finger domains may be engineered to target specific desired DNA sequences and enables zinc-finger nucleases to target unique sequences.

DNA-binding domains of individual ZFNs usually contain between three and six individual zinc finger repeats. Each zinc finger repeat may recognize between 9 and 18 nucleotide basepairs. In embodiments when the zinc finger domains recognize a 3 basepair DNA sequence they can generate a three-finger array that can recognize a nine basepair target site. In other embodiments, zinc finger domains may use one-finger or two-finger modules to generate zinc-finger arrays with six or more individual zinc fingers.

In various embodiments, a non-specific cleavage domain from a type IIs restriction endonuclease FokI may be used as a cleavage domain for a ZFN. The cleavage domain must dimerize in order to cleave DNA and therefore a pair of ZFNs may be required to target non-palindromic DNA sites. In various embodiments, ZFNs may fuse a cleavage domain to a C-terminus of each zinc finger domain. In various embodiments, the two individual ZFNs must bind opposite strands of DNA with their C-termini a certain distance apart in order to dimerize. In various embodiments, a linker sequence connects the zinc finger domain to the cleavage domain. In various embodiments, the linker sequence may separate the zinc finder domain from the cleavage domain by between about five to about seven basepairs.

In various embodiments, the CD38 expression modification may use CRISPR interference (CRISPRi). In various embodiments, CRISPRi may be used to knock-down gene expression of CD38 in a T cell. CRISPRi includes a genetic perturbation technique that allows for sequence-specific repression of gene expression in prokaryotic and eukaryotic cells as applied herein. In various embodiments, CRISPRi can sterically repress transcription by blocking either transcriptional initiation or elongation. In various embodiments, an sgRNA may be designed to be complementary to a promoter sequence or an exonic sequences. In various embodiments, the level of transcriptional repression of a target within a coding sequence may be strand-specific. In various embodiments, a CRISPR effector may be selected for stronger or weaker repression. In various embodiments, a dCas9 repressor may be stronger when a guide RNA is complementary to the non-template strand. In various embodiments, a dCas9 repressor may be weaker when a guide RNA is complementary to the template strand.

In various embodiments, the CD38 expression modification may comprise a single amino acid substitution. In various embodiments, a base editor described herein or elsewhere may be used to generate a single basepair substitution in a CD38 gene. In various embodiments, the single basepair substitution may lead to a transcriptional change resulting in an amino acid substation. In various embodiments, the single amino acid substitution may occur at an epitope. In various embodiments, the substitution replaces serine 274. In various embodiments, the substitution replaces the serine with a phenylalanine. In various embodiments, the substitution replaces threonine 116. In various embodiments, the substitution replaces the threonine with an alanine.

In various embodiments, the engineered T cell may include a CAR T cell. Techniques described herein and elsewhere are suitable for generating engineered T cells including a CD38 modification. In various embodiments, an early step in a production process for CAR T cells may include isolating T cells from human blood. However, the source of human blood may be the same or different than the patient being treated. For example, in various embodiments, the CAR T cells may be an allogeneic therapeutic. For example, a T cell donor may be different than a CAR T cell recipient where the CAR T cells are derived from the donor T cells. In various embodiments, the CAR T cells may be an autologous therapeutic. For example, a T cell donor may be the same as a CAR T cell recipient where the CAR T cells are derived from the donor T cells. T cells are then modified, expanded, and administered to a patient.

In various embodiments, CAR T cells target CD20. In various embodiments, the CAR T cell may include antigen binding molecules that may specifically bind to a CD20 antigen.

CD20 is a general B-cell marker expressed by most B cells and is encoded by MS4A1 gene. In healthy cells, CD20 may help enable optimal B cell immune response against T independent antigens. CD20 may be found on or overexpressed on B-cell lymphomas, hairy cell leukemia, B-cell chronic lymphocytic leukemia, and melanoma cancer stem cells. As such, CAR T cells may be optimized to target and destroy cells from B-cell lymphomas, hairy cell leukemia, B-cell chronic lymphocytic leukemia, and melanoma cancer stem cells in accordance with various embodiments. CD20 positive cells may be found in cases of Hodgkins disease, myeloma, and thymoma making the CAR T cells described herein well suited for treating those diseases as well in accordance with various embodiments. In various embodiments, the combination therapy may include a CD38 targeting molecule and a CD20 targeting CAR T cell.

In various embodiments, CAR T cells target CD19. In various embodiments, the CAR T cell may include antigen binding molecules that may specifically bind to a CD19 antigen. B-lymphocyte antigen CD19 (also called CD19 molecule (Cluster of Differentiation 19), B-Lymphocyte Surface Antigen B4, T-Cell Surface Antigen Leu-12 and CVID3) is encoded by the CD19 gene and may be expressed in B cells. CD19 may be involved in decisions to live, proliferate, differentiate, or die are continuously being made during B cell development. CD19 has been used to diagnose B cell lymphomas, acute lymphoblastic leukemia (ALL), and chronic lymphocytic leukemia (CLL). The majority of B cell malignancies express normal to high levels of CD19. As such, in various embodiments, the CAR T cells described herein may target and/or be optimized to target and destroy malignant B cells. In various embodiments, the combination therapy may include a CD38 targeting molecule and a CD19 targeting CAR T cell.

In various embodiments, CAR T cells target CLL-1. In various embodiments, the CAR T cell may include antigen binding molecules that may specifically bind to a CLL-1 antigen. In various embodiments, antigen binding molecules targeting CLL-1 may be used to treat acute myeloid leukemia therapy and/or any other disease or malignancy. Additional targets of CAR T cells for AML may include CD33, CD123, CLL-1, CD47, CD70, TIM3, or any combination thereof. In various embodiments, the combination therapy may include a CD38 targeting molecule and a CLL-1 targeting CAR T cell.

In various embodiments, CAR T cells target B-cell maturation antigen (BCMA). In various embodiments, the CAR T cell may include antigen binding molecules that may specifically bind to a BCMA antigen.

BCMA, also known as tumor necrosis factor superfamily member 17 (TNFRSF1 7) (UniProt Q02223), is a cell surface receptor exclusively expressed on plasma cells and plasmablasts. BCMA is a receptor for two ligands in the tumor necrosis factor (TNF) superfamily: APRIL (a proliferation inducing ligand, also known as TNFSF13; TALL-2 and TRDL-1; the high affinity ligand for BCMA) and B cell activation factor (BAFF) (also known as BLyS; TALL-I; THANK; zTNF4; TNFSF20; and D8Ertd387e; the low affinity ligand for BCMA). APRIL and BAFF are growth factors that bind BCMA and promote survival of plasma cells. BCMA is also highly expressed on malignant plasma cells in human multiple myeloma (MM). Antibodies binding to BCMA are described, for example, in Gras et al., 1995, Int. Immunol. 7:1093-1106, WO200124811 and WO200124812. Anti-BCMA antibodies that cross-react with TACI are described in WO2002/066516. Bispecific antibodies against BCMA and CD3 are described, for example, in US 2013/0156769 A1 and US 2015/0376287 A1. An anti-BCMA antibody-MMAE or -MMAF conjugate has been reported to selectively induce killing of multiple myeloma cells (Tai et al., Blood 2014, 123 (20): 3128-38). Ali et al., Blood 2016, 128 (13): 1688-700, have reported that in a clinical trial (#NCT02215967) chimeric antigen receptor (CAR) T cells targeting BCMA resulted in remission of multiple myeloma in human patients. In various embodiments, any BCMA therapy described herein or elsewhere may be included in a combination therapy. More specifically, CAR T cells for targeting BCMA may be combined with a CD38 targeting compound to generate a combination therapy product.

In various embodiments, CAR T cells target epidermal growth factor receptor (EGFR). In various embodiments, the CAR T cell may include antigen binding molecules that may specifically bind to an EGFR antigen.

EGFR is a transmembrane protein that is a receptor for members of the epidermal growth factor family (EGF family) of extracellular protein ligands. In healthy cells, EGFR is activated by binding of its specific ligands, including epidermal growth factor and transforming growth factor alpha (TGF-α). The EGFR is essential for ductal development of the mammary glands. As such, mutations that lead to EGFR overexpression have been associated with variety of cancers types. Cancer types include adenocarcinoma of the lung, anal cancers, glioblastoma and epithelian tumors of the head and neck. These somatic mutations involving EGFR lead to its constant activation, which produces uncontrolled cell division. As such, a combination therapy may include antigen binding molecules for targeting EGFR in accordance with various embodiments. In various embodiments, the antigen binding molecules may form all or a portion of a cell surface protein for a CAR T cell. In various embodiments, the combination therapy may include a CD38 targeting molecule and an EGFR targeting CAR T cell.

In various embodiments, CAR T cells target HER2. In various embodiments, the CAR T cell may include antigen binding molecules that may specifically bind to a HER2 antigen. Receptor tyrosine-protein kinase erbB-2 is normally a membrane-bound protein of cells. erbB-2 is encoded by the ERBB2 gene. HER2 is a member of the human epidermal growth factor receptor (HER/EGFR/ERBB) family and its signaling pathways include mitogen-activated protein kinase (MAPK), phosphoinositide 3-kinase (PI3K/Akt), phospholipase C γ, protein kinase C (PKC), and signal transducer and activator of transcription (STAT).

Over-expression of the ERBB2 gene commonly occurs in breast cancers and HER2-positive breast cancers are well established as being associated with increased disease recurrence and a poor prognosis. As such, antigen binding molecules described herein may be used to target ERBB2 and more specifically HER2 in accordance with various embodiments. In various embodiments, CAR T cells may be engineered to express antigen binding molecules specific for ERBB2 and more specifically HER2. In various embodiments, the combination therapy may include a CD38 targeting molecule.

In various embodiments, CAR T cells target Glypican-3 (GPC3). In various embodiments, the CAR T cell may include antigen binding molecules that may specifically bind to a GPC3 antigen. GPC3 is a protein that is encoded by the GPC3 gene. GPC3 may play a role in the control of cell division and growth regulation. Upregulation of GPC3 can be found in cancer types such as liver cancer. As such, antigen binding molecules may be used to target GPC3 in accordance with various embodiments. In various embodiments, CAR T cells may be engineered to expression antigen binding molecules specific for GPC3. In various embodiments, the antigen binding molecules and/or the CAR T cells may be used in a combination therapy. In various embodiments, the combination therapy may include a CD38 targeting molecule.

In various embodiments, CAR T cells target GD2. In various embodiments, the CAR T cell may include antigen binding molecules that may specifically bind to a GD2 antigen.

In various embodiments, an antigen binding molecule of a CAR T cell may include a binding motif that binds an antigen selected from the group comprising 5T4, alphafetoprotein, B cell maturation antigen (BCMA), B cell receptor, CA-125, carcinoembryonic antigen, CD19, CD20, CD22, CD23, CD30, CD33, CD40, CD56, CD79, CD78, CD123, CD138, c-Met, CSPG4, IgM, C-type lectin-like molecule 1 (CLL-1), EGFRvIII, epithelial tumor antigen, ERBB2, FLT3, folate binding protein, GD2, GD3, HER1-HER2 in combination, HER2-HER3 in combination, HER2/Neu, HERV-K, HIV-1 envelope glycoprotein gp41, HIV-1 envelope glycoprotein gpl20, IL-IIRalpha, kappa chain, lambda chain, melanoma-associated antigen, mesothelin, MUC-1, mutated p53, mutated ras, prostate-specific antigen, ROR1, VEGFR2, EphA3 (EPH receptor A3), BAFFR (B-cell activating factor receptor), and combinations thereof.

In various embodiments, the engineered T cells may comprise CAR molecules. In various embodiments, CAR molecules may comprise any anti-CD19 CAR molecule. In various aspects the anti-CD19 CAR may include an extracellular scFv domain, an intracellular and/or transmembrane, portion of a CD28 molecule, an optional extracellular portion of the CD28 molecule, and an intracellular CD3zeta domain as described in WO2015120096 or WO2016191755, each of which is incorporated herein in its entirety.

In various embodiments, the anti-CD19 CAR may also comprise additional domains, such as a CD8 extracellular and/or transmembrane region, an extracellular immunoglobulin Fc domain (e.g., IgG1, IgG2, IgG3, IgG4), or one or more additional signaling domains, such as 41 BB, OX40, CD2 CD16, CD27, CD30 CD40, PD-1, ICOS, LFA-1, IL-2 Receptor, Fc gamma receptor, or any other costimulatory domains with immunoreceptor tyrosine-based activation motifs.

In various embodiments, the cell surface receptor may comprise an anti-CD19 CAR, such as FMC63-28Z CAR or FMC63-CD828BBZ CAR as set forth in Kochenderfer et al., J Immunother. 2009 September; 32 (7): 689-702, “Construction and Preclinical Evaluation of an Anti-CD19 Chimeric Antigen Receptor,” the subject matter of which is hereby incorporated by reference for the purpose of providing the methods of constructing the vectors used to produce T cells expressing the FMC63-28Z CAR or FMC63-CD828BBZ CAR.

In various embodiments, the T cell that comprises a CAR molecule may be Yescarta® (axicabtagene ciloleucel). In various embodiments, the T cell that includes a CAR molecule may be Tecartus® (brexucabtagene autoleucel).

In various embodiments, the engineered T cells may comprise a dual-targeted antigen binding system. Dual-targeted antigen binding systems may comprise bispecific CARs or TCRs and/or bicistronic CARs or TCRs. Bispecific and bicistronic CARs may comprise two binding motifs (in a single CAR molecule or in two CAR molecules, respectively). In various embodiments, the vector of the present disclosure may encode bicistronic and/or bispecific CARs (e.g., bicistronic and/or bispecific CARs that bind CD20 and CD19). In various embodiments, the bispecific CAR may comprise one which targets CD19 and CD20 as described in WO2020123691, which is incorporated herein in its entirety.

### IV. CD38 and CD38 Targeting Therapies

### i) CD38

Cluster of differentiation 38 (CD38), also known as cyclic ADP ribose hydrolase is a glycoprotein expressed in a variety of different immune cells including natural killer cells, B cells, dendritic cells, activated T cells, Tregs, platelets, and red blood cells (e.g., see FIG. 1). For this reason, the embodiments described herein may be particularly well suited to combination therapies where a co-therapeutic may be subject to NK cell depletion and require protectionary measure.

“CD38, primarily a NAD+ glycohydrolase and ADPR cyclase, is a multifunctional transmembrane protein whose abnormal overexpression in a variety of tumor types is associated with cancer progression. It is linked to VEGFR2 mediated angiogenesis and immune suppression as it favors the recruitment of suppressive immune cells like Tregs and myeloid-derived suppressor cells, thus helping immune escape. CD38 is expressed in MI macrophages and in neutrophil and T cell-mediated immune response and is associated with IFNc-mediated suppressor activity of immune responses. Targeting CD38 with anti-CD38 monoclonal antibodies in hematological malignancies has shown excellent results.” Abstract, Dwivedi S, Rendón-Huerta EP, Ortiz-Navarrete V, Montaño LF. CD38 and Regulation of the Immune Response Cells in Cancer. J Oncol. 2021 Feb. 27; 2021:6630295. doi: 10.1155/2021/6630295. PMID: 33727923; PMCID: PMC7936891 the disclosure of which is hereby incorporated by reference in its entirety for all purposes.

CD38 is overexpressed in variety of hematologic malignancies such as acute myeloid leukemia (AML), multiple myeloma (MM), diffuse large B cell lymphoma (DLBCL), adult acute lymphoblastic leukemia (ALL), T cell ALL, mantle cell lymphoma (MCL), follicular lymphoma (FL), and chronic lymphocytic leukemia (CLL). For this reason, the embodiments described herein may be particularly well suited to use as treatments for AML, MM, DLBCL, ALL, T cell ALL, MCL, FL, and CLL.

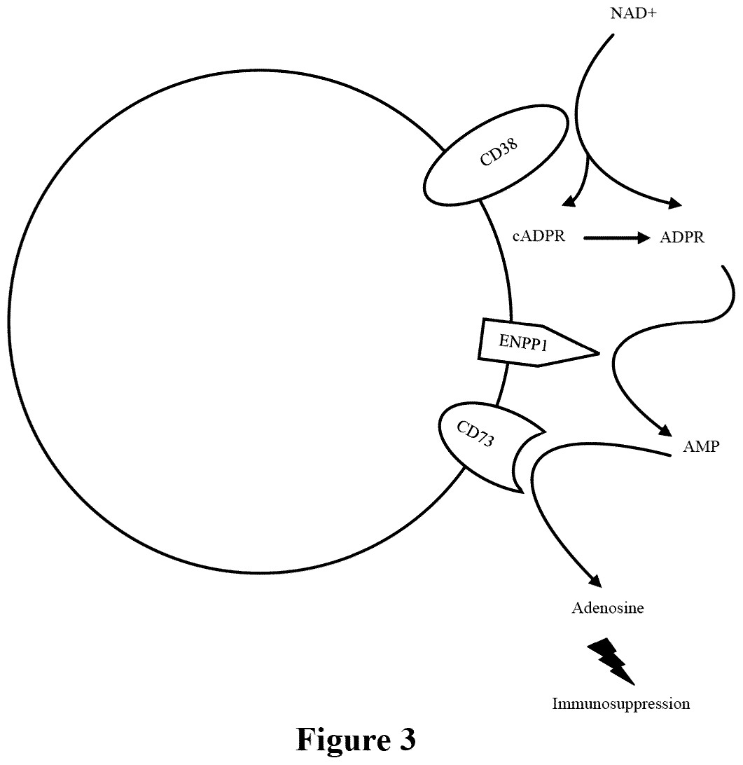

FIG. 3 is a schematic diagram of a cell expressing components of the adenosine biosynthesis pathway. CD38 is the first enzyme in the pathway responsible for conversion of NAD+ to ADPR. Literature suggests that upregulation in multiple myeloma cells leads to a cascade of events eventually causing immunosuppression in the localized area (e.g., within or near a tumor mass). The technology described herein takes CD38 expression levels into account on various cell types, including, CD38 expression levels on cancer cells (e.g., multiple myeloma). For example, the systems and methods described herein allow specific targeting of CD38 antigens as well as CAR T cell evasion from CD38 targeting compounds and/or NK cells.

An immunosuppression pathway may include CD38, ENPP1, and CD73 protein activity. More specifically, an immunosuppression pathway may include over expression of CD38, thereby, increasing the cascade of reactions generating adenosine.

As shown in FIG. 3, CD38 may function as an enzyme that catalyzes the synthesis of ADP ribose (ADPR) (97%) and cyclic ADP-ribose (cADPR) (3%) from NAD+. CD38 is thought to be a major regulator of NAD+ levels, its NADase activity is much higher than its function as an ADP-rybosyl-cyclase: for every 100 molecules of NAD+ converted to ADP ribose it generates one molecule of cADPR. When nicotinic acid is present under acidic conditions, CD38 can hydrolyze nicotinamide adenine dinucleotide phosphate (NADP+) to NAADP. See Kar A, et al. CD38: T Cell Immuno-Metabolic Modulator. Cells. 2020 Jul. 17; 9 (7): 1716. doi: 10.3390/cells9071716. PMID: 32709019; PMCID: PMC7408359 and Braidy N, et al., Role of Nicotinamide Adenine Dinucleotide and Related Precursors as Therapeutic Targets for Age-Related Degenerative Diseases: Rationale, Biochemistry, Pharmacokinetics, and Outcomes. Antioxid Redox Signal. 2019 Jan. 10; 30 (2): 251-294. doi: 10.1089/ars.2017.7269. Epub 2018 May 11. PMID: 29634344; PMCID: PMC6277084.

Ectonucleotide pyrophosphatase/phosphodiesterase family member 1 (ENPP1, PC-1, CD203a) is an enzyme encoded by the ENPP1 gene. ENPP1 is a type II transmembrane glycoprotein. Adenosine triphosphate (ATP) is the primary substrate of ENNP1, which is cleaved into adenosine monophosphate (AMP) and diphosphate. Additionally, ENNP1 can hydrolyze nicotinamide adenine dinucleotide (NAD+) to produce AMP. Further, ADPR can also be hydrolyzed by ENNP1 to generate AMP.

Once AMP is generated it can then be converted into adenosine. For example, 5′-nucleotidase (5′-NT), also known as ecto-5′-nucleotidase or CD73 (cluster of differentiation 73), is an enzyme that in humans is encoded by the NT5E gene that converts AMP to adenosine. CD73 is a surface enzyme which is expressed on multiple cells (e.g., multiple myeloma cells). CD73 mediates the hydrolysis of the autocrine and paracrine danger signals of ATP and ADP to anti-inflammatory adenosine. Immune suppression mediated by adenosinergic pathways is involved in maintaining immune system homeostasis. Immune suppressive functions of T regulatory cells are also dependent on CD73 expression.

CD38 is overexpressed in multiple myelomas where malignant plasma cells overexpressing CD38 accumulate in the bone marrow. Multiple myeloma cells can upregulate antiapoptotic molecules such as survivin within the bone marrow microenvironment to protect against CD38 antibody-induced cellular cytotoxicity. Further, CD38 increases phosphorylation of phosphoinositide 3-kinase (PI3K), protein kinase B (PKB, AKT), and mammalian target of rapamycin complex (mTORC) which upregulates the PI3K/AKT/mTOR pathway. Upregulation of PI3K/AKT/mTOR pathway is related to metabolic reprogramming and proliferation of cancer cells. mTOR and rapamycin-insensitive companion of mammalian target of rapamycin (RICTOR) are overexpressed in multiple myeloma endothelial cells and mTOR complex 2 (mTORC2) and its associated downstream effectors are linked with an angiogenic switch to multiple myeloma. As such, inhibition of the PI3K/AKT/mTOR pathway with one or more CD38 targeting compounds (a dual mTOR inhibitor along with anti-CD38 therapies) in combination with one or more of the engineered T cells described herein may exhibit a synergistic effect in CD38-expressing multiple myeloma cells.

In various embodiments, the use of CD38 targeting compounds (e.g., anti-CD38 mAbs) may deplete CD38+ myeloid-derived suppressor cells (MDSCs), T regs, and B regs immune suppressive cells. In various embodiments, the use of CD38 targeting compounds may enhance antitumor activity.

In melanoma cells, CD38 consumes NAD through adenosine pathway and finally generates aldehyde deformylating oxygenases (ADO) to inhibit T cell proliferation. This inhibition is more effective for T cell subsets (e.g, CD4+ T cells and CD8+ T cells) that are involved in an anti-tumor immune response. In various embodiments, engineered T cells described herein may be engineered to target ADO. In various embodiments, a combination therapy may include a CD38 targeting compound and an engineered T cell including a CD38 expression modification as described herein.

### ii) CD38 Targeting Therapies

In various embodiments, the technologies described herein include a CD38 targeting compound. For example, a CD38 targeting compound may be used as a co-therapy treatment along with one or more of the engineered T cells described herein. In various embodiments, an engineered T cell including a CD38 expression modification and a CD38 targeting compound may be incorporated into a cell therapy product. In various embodiments, the cell therapy product may be administered to a patient.

In various embodiments, the CD38 targeting compound comprises an antigen binding molecule having specificity for a CD38 antigen. In various embodiments, the antigen binding molecule may include anti-CD38 antibodies. In various embodiments, the antigen binding molecule may include a DARPin. In various embodiments, the antigen binding molecule may include a nano-body. In various embodiments, the antigen binding molecule may include a ligand trap. In various embodiments, the antigen binding molecule may include a synthetic component.

In various embodiments, the CD38 targeting compound comprises isatuximab. In various embodiments, the CD38 targeting compound comprises daratumumab. In various embodiments, the CD38 targeting compound MOR202. In various embodiments, the CD38 targeting compound comprises TAK079.

### V. Exemplary Systems for a Combination Therapy

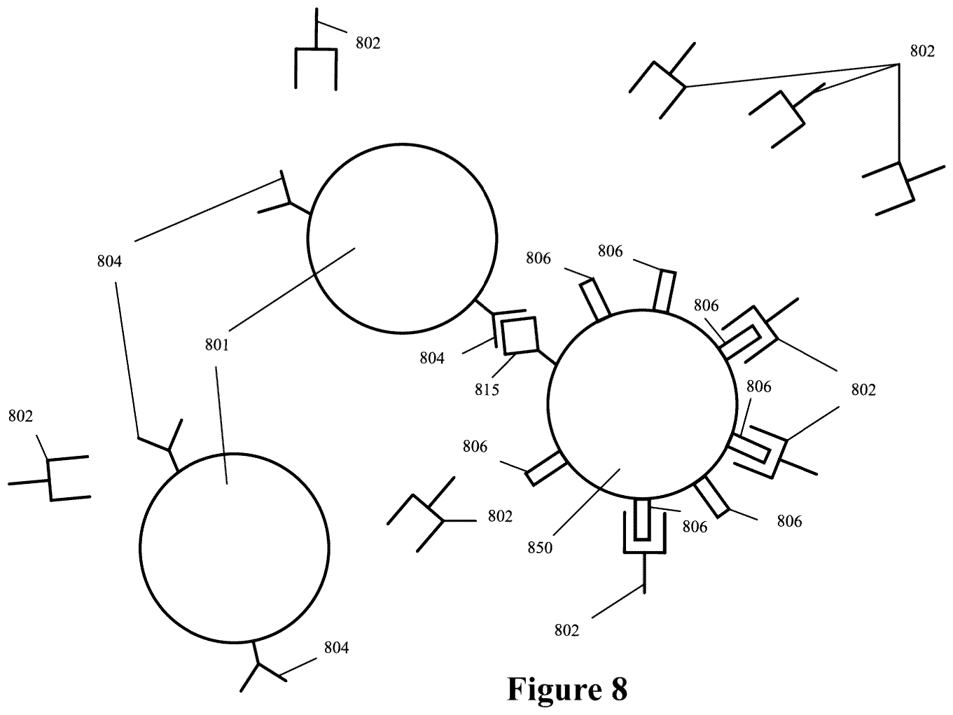

FIG. 8 is an illustration of a system in which a CD38 targeting compound 802 is targeting CD38 antigens 806 of a target cell 850 (non-limiting examples include a cancer cell, a multiple myeloma cell, etc.) 850 and engineered T cells 801 are protected by a CD38 expression modification in accordance with various embodiments. In this example, a chimeric antigen receptor 804 is shown bound to a target 815 (e.g., CD19, CD20, or any other target described herein or elsewhere).

In various embodiments, a CD38 expression modification may result in complete inhibition, partial inhibition, a phenotypic change, and increase in expression, or any conceivable expression change or combination thereof. In various embodiments, a system such as the one illustrated in FIG. 8 may be the result of a knock-out of a CD38 gene. Complete inhibition may be achieved in various other ways described both herein and elsewhere according to various embodiments.

In various systems, an engineered T cell 801 may express a cell surface protein 815 such as chimeric antigen receptor that may recognize a cell surface protein 815 on a target cell 850 (e.g., a cancer cell). In the same system, CD38 targeting compounds 802 may also target a CD38 antigen 806 on a target cell 850. Numerous synergistic effects are detailed herein, and numerous protections are detailed as well.

In various embodiments, the engineered T cells 801 may target include CD20, CD19, CLL-1, BCMA, EGFR, HER2, GPC3, GD2, or any combination thereof. In various embodiments, the engineered T cells 801 may comprise an antigen binding molecule including a binding motif that binds an antigen selected from the group comprising 5T4, alphafetoprotein, B cell maturation antigen (BCMA), B cell receptor, CA-125, carcinoembryonic antigen, CD19, CD20, CD22, CD23, CD30, CD33, CD40, CD56, CD79, CD78, CD123, CD138, c-Met, CSPG4, IgM, C-type lectin-like molecule 1 (CLL-1), EGFRvIII, epithelial tumor antigen, ERBB2, FLT3, folate binding protein, GD2, GD3, HER1-HER2 in combination, HER2-HER3 in combination, HER2/Neu, HERV-K, HIV-1 envelope glycoprotein gp41, HIV-1 envelope glycoprotein gpl20, IL-IIRalpha, kappa chain, lambda chain, melanoma-associated antigen, mesothelin, MUC-1, mutated p53, mutated ras, prostate-specific antigen, ROR1, VEGFR2, EphA3 (EPH receptor A3), BAFFR (B-cell activating factor receptor), and combinations thereof.

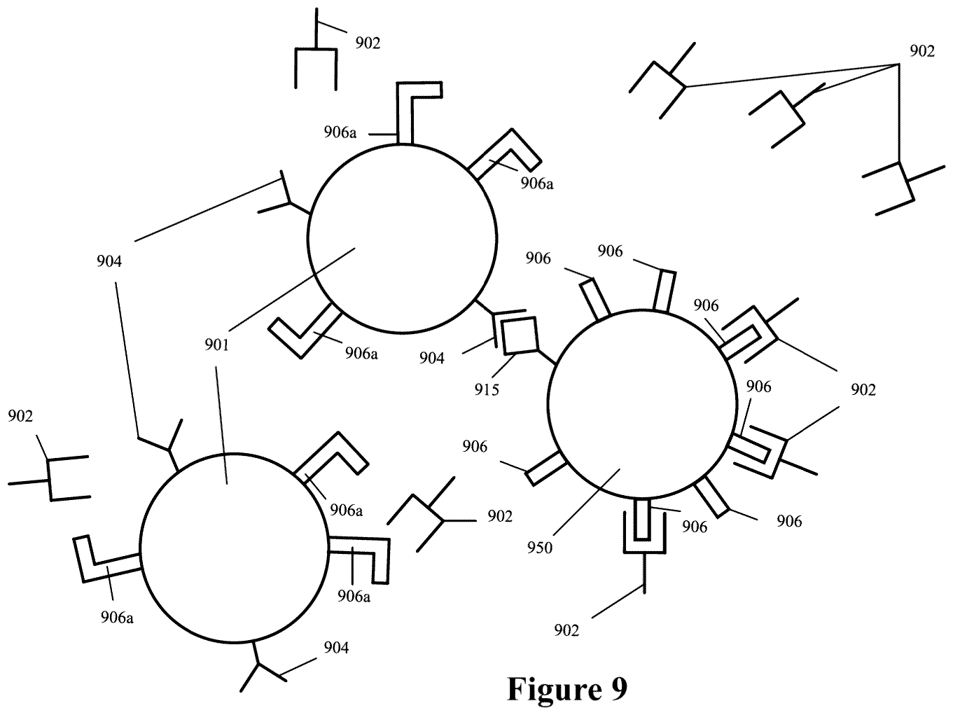

FIG. 9 is an illustration of a system in which CD38 targeting compound 902 is targeting CD38 antigens 906 on a target cell 950 (non-limiting examples include a cancer cell, a multiple myeloma cell, etc.) and engineered T cells 901 are protected by a CD38 expression modification 906a in accordance with various embodiments. Exemplary embodiments may include point mutations occurring at an epitope in an antigen as the expression modification 906a and/or receptor molecule that inhibit binding of a CD38 targeting compound 902. In such systems, both the CD38 targeting compound 902 and the chimeric antigen receptor 904 target the target cell 905 through a CAR specific antigen 915 and CD38 antigen 906.

In various embodiments, the engineered T cells 901 may target include CD20, CD19, CLL-1, BCMA, EGFR, HER2, GPC3, GD2, or any combination thereof. In various embodiments, the engineered T cells 901 may comprise an antigen binding molecule including a binding motif that binds an antigen selected from the group comprising 5T4, alphafetoprotein, B cell maturation antigen (BCMA), B cell receptor, CA-125, carcinoembryonic antigen, CD19, CD20, CD22, CD23, CD30, CD33, CD40, CD56, CD79, CD78, CD123, CD138, c-Met, CSPG4, IgM, C-type lectin-like molecule 1 (CLL-1), EGFRvIII, epithelial tumor antigen, ERBB2, FLT3, folate binding protein, GD2, GD3, HER1-HER2 in combination, HER2-HER3 in combination, HER2/Neu, HERV-K, HIV-1 envelope glycoprotein gp41, HIV-1 envelope glycoprotein gpl20, IL-IIRalpha, kappa chain, lambda chain, melanoma-associated antigen, mesothelin, MUC-1, mutated p53, mutated ras, prostate-specific antigen, ROR1, VEGFR2, EphA3 (EPH receptor A3), BAFFR (B-cell activating factor receptor), and combinations thereof.

Various systems for a combination therapy using a T cell and a CD38 targeting compound are described herein. For example, a system may comprise an engineered T cell including a CD38 expression modification and a CD38 targeting compound.

In various embodiments, the CD38 targeting compound comprises an antigen binding molecule having specificity for a CD38 antigen. In various embodiments, the antigen binding molecule includes anti-CD38 antibodies. In various embodiments, the antigen binding molecule includes a DARPin. In various embodiments, the antigen binding molecule includes a nano-body. In various embodiments, the antigen binding molecule includes a ligand trap. In various embodiments, the antigen binding molecule includes a synthetic component. In various embodiments, the CD38 targeting compound comprises isatuximab. In various embodiments, the CD38 targeting compound comprises daratumumab. In various embodiments, the CD38 targeting compound comprises MOR202. In various embodiments, the CD38 targeting compound comprises TAK079.

In various embodiments, the CD38 expression modification comprises a CD38 gene knock-out. In various embodiments, the gene knock-out is Cas9 mediated. In various embodiments, the Cas9 mediated gene knock-out uses a complex including a Cas9 molecule and a guide nucleotide sequence. In various embodiments, the nucleotide guide sequence comprising SEQ ID NO: 1. In various embodiments, the nucleotide guide sequence comprising SEQ ID NO: 2. In various embodiments, the nucleotide guide sequence comprising SEQ ID NO: 3. In various embodiments, the nucleotide guide sequence comprising SEQ ID NO: 4. In various embodiments, the gene knock-out is Cas12 mediated.

In various embodiments, the gene knock-out is base editor mediated. In various embodiments, the base editor may include a cytosine base editor (CBE). In various embodiments, the cytosine base editor includes a cytidine base editor comprised of a Cas9 nickase fused to a cytidine deaminase derived from APOBEC1. In various embodiments, the base editor may include an adenine base editor (ABE). In various embodiments, the cytidine base editor may be comprised of Cas9 nickase fused to cytidine deaminase derived from APOBEC1. In various embodiments, the adenine base editor may be comprised of Cas9 nickase fused to deoxyadenosine deaminase derived TadA.

In various embodiments, the base editor may be complexed with a nucleotide guide sequence. In various embodiments, the nucleotide guide sequence comprising SEQ ID NO: 5. In various embodiments, the nucleotide guide sequence comprising SEQ ID NO: 6. In various embodiments, the nucleotide guide sequence comprising SEQ ID NO: 7. In various embodiments, the nucleotide guide sequence comprising SEQ ID NO: 8. In various embodiments, the nucleotide guide sequence comprising SEQ ID NO: 9. In various embodiments, the nucleotide guide sequence comprising SEQ ID NO: 10.

In various embodiments, a CD38 gene knock-in. In various embodiments, the CD38 gene knock-in uses homology directed repair.

In various embodiments, the CD38 expression modification uses RNAi. In various embodiments, the CD38 expression modification uses anti-sense oligonucleotide (ASO). In various embodiments, the CD38 expression modification uses epigenetic editing. In various embodiments, the epigenetic editing uses a TALEN. In various embodiments, the epigenetic editing uses a zinc finger nuclease. In various embodiments, the epigenetic editing uses CRISPRi.

In various embodiments, the CD38 expression modification comprises a single amino acid substitution. In various embodiments, the single amino acid substitution occurs at an epitope specific for Daratumumab. In various embodiments, the single amino acid substitution occurs at an epitope specific for Isatuximab. In various embodiments, the single amino acid substitution is mediated by a base editor. In various embodiments, the substitution replaces serine 274. In various embodiments, the substitution replaces the serine with a phenylalanine. In various embodiments, the base editor includes a nucleotide guide sequence comprising SEQ ID NO: 11.

In various embodiments, the substitution replaces threonine 116. In various embodiments, the substitution replaces the threonine with an alanine. In various embodiments, the base editor includes a nucleotide guide sequence comprising SEQ ID NO: 17. In various embodiments, the base editor includes a nucleotide guide sequence comprising SEQ ID NO: 16. In various embodiments, the base editor includes a nucleotide guide sequence comprising SEQ ID NO: 18. In various embodiments, the base editor includes a nucleotide guide sequence comprising SEQ ID NO: 19. In various embodiments, the base editor includes a nucleotide guide sequence comprising SEQ ID NO: 20.

In various embodiments, the engineered T cell expresses a CD38 antigen nucleotide encoding sequence comprising SEQ ID NO: 21. In various embodiments, the engineered T cell expresses a CD38 antigen protein molecule comprising SEQ ID NO: 23. In various embodiments, the engineered T cell expresses a CD38 antigen protein molecule comprising SEQ ID NO: 24.

In various embodiments, the engineered T cell includes a CAR T cell. In various embodiments, the system is an allogeneic therapeutic. In various embodiments, the system is an autologous therapeutic.

In various embodiments, the engineered T cell targets CD20. In various embodiments, the engineered T cell targets CD19. In various embodiments, the engineered T cell targets CLL-1. In various embodiments, the engineered T cell targets BCMA. In various embodiments, the engineered T cell targets EGFR. In various embodiments, the CAR T cells target HER2. In various embodiments, the engineered T cell targets GPC3. In various embodiments, the engineered T cell targets GD2.

In various embodiments, the engineered T cells may comprise CAR molecules. In various embodiments, CAR molecules may comprise any anti-CD19 CAR molecule. In various aspects the anti-CD19 CAR may include an extracellular scFv domain, an intracellular and/or transmembrane, portion of a CD28 molecule, an optional extracellular portion of the CD28 molecule, and an intracellular CD3zcta domain as described in WO2015120096 or WO2016191755, each of which is incorporated herein in its entirety.

In various embodiments, the anti-CD19 CAR may also comprise additional domains, such as a CD8 extracellular and/or transmembrane region, an extracellular immunoglobulin Fc domain (e.g., IgG1, IgG2, IgG3, IgG4), or one or more additional signaling domains, such as 41 BB, OX40, CD2 CD16, CD27, CD30 CD40, PD-1, ICOS, LFA-1, IL-2 Receptor, Fc gamma receptor, or any other costimulatory domains with immunoreceptor tyrosine-based activation motifs.

In various embodiments, the cell surface receptor may comprise an anti-CD19 CAR, such as FMC63-28Z CAR or FMC63-CD828BBZ CAR as set forth in Kochenderfer et al., J Immunother. 2009 September; 32 (7): 689-702, “Construction and Preclinical Evaluation of an Anti-CD19 Chimeric Antigen Receptor,” the subject matter of which is hereby incorporated by reference for the purpose of providing the methods of constructing the vectors used to produce T cells expressing the FMC63-28Z CAR or FMC63-CD828BBZ CAR.

In various embodiments, the T cell that comprises a CAR molecule may be Yescarta® (axicabtagene ciloleucel). In various embodiments, the T cell that includes a CAR molecule may be Tecartus® (brexucabtagene autoleucel).

In various embodiments, the engineered T cells may comprise a dual-targeted antigen binding system. Dual-targeted antigen binding systems may comprise bispecific CARs or TCRs and/or bicistronic CARs or TCRs. Bispecific and bicistronic CARs may comprise two binding motifs (in a single CAR molecule or in two CAR molecules, respectively). In various embodiments, the vector of the present disclosure may encode bicistronic and/or bispecific CARs (e.g., bicistronic and/or bispecific CARs that bind CD20 and CD19). In various embodiments, the bispecific CAR may comprise one which targets CD19 and CD20 as described in WO2020123691, which is incorporated herein in its entirety.

### VI. Exemplary Methods of Generating A Cell Therapy Treatment

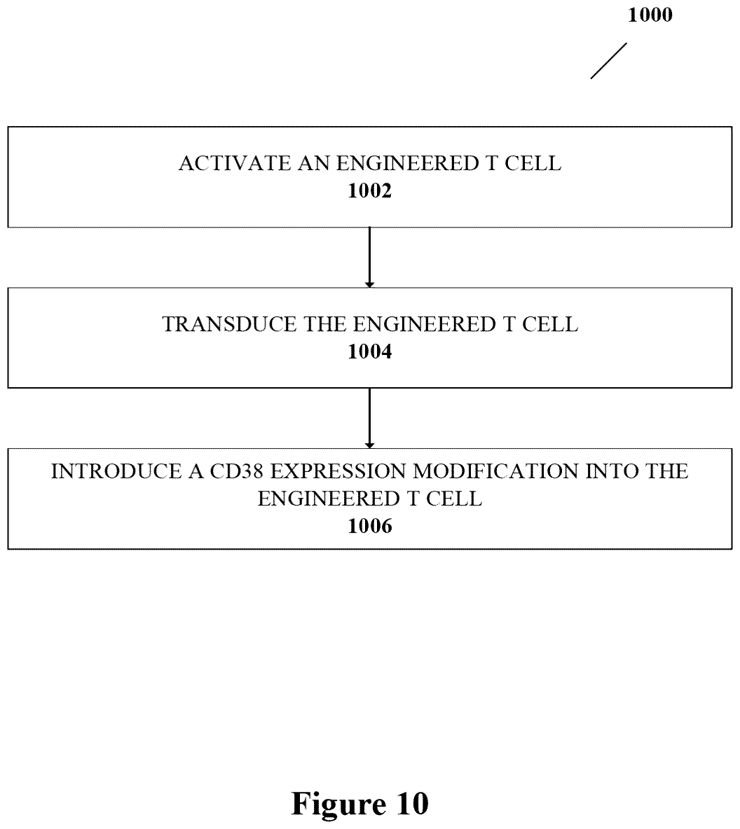

FIG. 10 illustrates a process for generating a cell therapy treatment 1000 in accordance with various embodiments.

Step 1002 includes activating an engineered T cell according to various embodiments.

Step 1004 includes transducing the engineered T cell. In various embodiments, the transducing step uses a lentiviral vector according to various embodiments.

Step 1006 includes introducing a CD38 expression modification into an engineered T cell.

In various embodiments, expanding the engineered T cell according to various embodiments.

In various embodiments, the CD38 expression modification comprises a CD38 gene knock-out. In various embodiments, the gene knock-out is Cas9 mediated. In various embodiments, the Cas9 mediated gene knock-out uses a complex including a Cas9 molecule and a guide nucleotide sequence. In various embodiments, the nucleotide guide sequence comprising SEQ ID NO: 1. In various embodiments, the nucleotide guide sequence comprising SEQ ID NO: 2. In various embodiments, the nucleotide guide sequence comprising SEQ ID NO: 3. In various embodiments, the nucleotide guide sequence comprising SEQ ID NO: 4.

In various embodiments, the engineered T cell may target include CD20, CD19, CLL-1, BCMA, EGFR, HER2, GPC3, GD2, or any combination thereof. In various embodiments, the engineered T cell may comprise an antigen binding molecule including a binding motif that binds an antigen selected from the group comprising 5T4, alphafetoprotein, B cell maturation antigen (BCMA), B cell receptor, CA-125, carcinoembryonic antigen, CD19, CD20, CD22, CD23, CD30, CD33, CD40, CD56, CD79, CD78, CD123, CD138, c-Met, CSPG4, IgM, C-type lectin-like molecule 1 (CLL-1), EGFRvIII, epithelial tumor antigen, ERBB2, FLT3, folate binding protein, GD2, GD3, HER1-HER2 in combination, HER2-HER3 in combination, HER2/Neu, HERV-K, HIV-1 envelope glycoprotein gp41, HIV-1 envelope glycoprotein gpl20, IL-IIRalpha, kappa chain, lambda chain, melanoma-associated antigen, mesothelin, MUC-1, mutated p53, mutated ras, prostate-specific antigen, ROR1, VEGFR2, EphA3 (EPH receptor A3), BAFFR (B-cell activating factor receptor), and combinations thereof.

In various embodiments, the gene knock-out is Cas12 mediated.

In various embodiments, the gene knock-out is base editor mediated. In various embodiments, the base editor includes a cytosine base editor (CBE). In various embodiments, the cytosine base editor includes a cytidine base editor comprised of a Cas9 nickase fused to a cytidine deaminase derived from APOBEC1.

In various embodiments, the base editor includes an adenine base editor (ABE).

In various embodiments, the base editor is complexed with a nucleotide guide sequence. In various embodiments, the nucleotide guide sequence comprising SEQ ID NO: 5. In various embodiments, the nucleotide guide sequence comprising SEQ ID NO: 6. In various embodiments, the nucleotide guide sequence comprising SEQ ID NO: 7. In various embodiments, the nucleotide guide sequence comprising SEQ ID NO: 8. In various embodiments, the nucleotide guide sequence comprising SEQ ID NO: 9. In various embodiments, the nucleotide guide sequence comprising SEQ ID NO: 10.

In various embodiments, a CD38 gene knock-in. In various embodiments, the CD38 gene knock-in uses homology directed repair.

In various embodiments, the CD38 expression modification uses RNAi. In various embodiments, the CD38 expression modification uses anti-sense oligonucleotide (ASO).

In various embodiments, the CD38 expression modification uses epigenetic editing. In various embodiments, the epigenetic editing uses a TALEN. In various embodiments, the epigenetic editing uses a zinc finger nuclease.

In various embodiments, the epigenetic editing uses CRISPRi. In various embodiments, the CD38 expression modification comprises a single amino acid substitution. In various embodiments, the single amino acid substitution occurs at an epitope specific for Daratumumab.

In various embodiments, the single amino acid substitution occurs at an epitope specific for Isatuximab. In various embodiments, the single amino acid substitution is mediated by a base editor.

In various embodiments, the substitution replaces serine 274. In various embodiments, the substitution replaces the serine with a phenylalanine. In various embodiments, the base editor includes a nucleotide guide sequence comprising SEQ ID NO: 17.

In various embodiments, the substitution replaces threonine 116. In various embodiments, the substitution replaces the threonine with an alanine. In various embodiments, the base editor includes a nucleotide guide sequence comprising SEQ ID NO: 16. In various embodiments, the base editor includes a nucleotide guide sequence comprising SEQ ID NO: 18. In various embodiments, the base editor includes a nucleotide guide sequence comprising SEQ ID NO: 19. In various embodiments, the base editor includes a nucleotide guide sequence comprising SEQ ID NO: 20.

In various embodiments, the engineered T cell expresses a CD38 antigen nucleotide encoding sequence comprising SEQ ID NO: 21. In various embodiments, the engineered T cell expresses a CD38 antigen protein molecule comprising SEQ ID NO: 23. In various embodiments, the engineered T cell expresses a CD38 antigen protein molecule comprising SEQ ID NO: 24. In various embodiments, the engineered T cell includes a CAR T cell.

In various embodiments, the system is an allogeneic therapeutic. In various embodiments, the system is an autologous therapeutic.

In various embodiments, the engineered T cell targets CD20. In various embodiments, the engineered T cell targets CD19. In various embodiments, the engineered T cell targets CLL-1. In various embodiments, the engineered T cell targets BCMA. In various embodiments, the engineered T cell targets EGFR. In various embodiments, the CAR T cells target HER2. In various embodiments, the engineered T cell targets GPC3. In various embodiments, the engineered T cell targets GD2.

In various embodiments, the method further comprises combining the CAR T cells with a CD38 targeting compound 1010 to generate a cell therapy product.

In various embodiments, the CD38 targeting compound comprises an antigen binding molecule having specificity for a CD38 antigen. In various embodiments, the antigen binding molecule includes anti-CD38 antibodies. In various embodiments, the antigen binding molecule includes a DARPin. In various embodiments, the antigen binding molecule includes a nano-body. In various embodiments, the antigen binding molecule includes a ligand trap. In various embodiments, the antigen binding molecule includes a synthetic component.

In various embodiments, the CD38 targeting compound comprises isatuximab. In various embodiments, the CD38 targeting compound comprises daratumumab. In various embodiments, the CD38 targeting compound comprises MOR202. In various embodiments, the CD38 targeting compound comprises TAK079.

### VII. Exemplary Methods of Treating A Patient Using A T Cell and A CD38 Targeting Compound

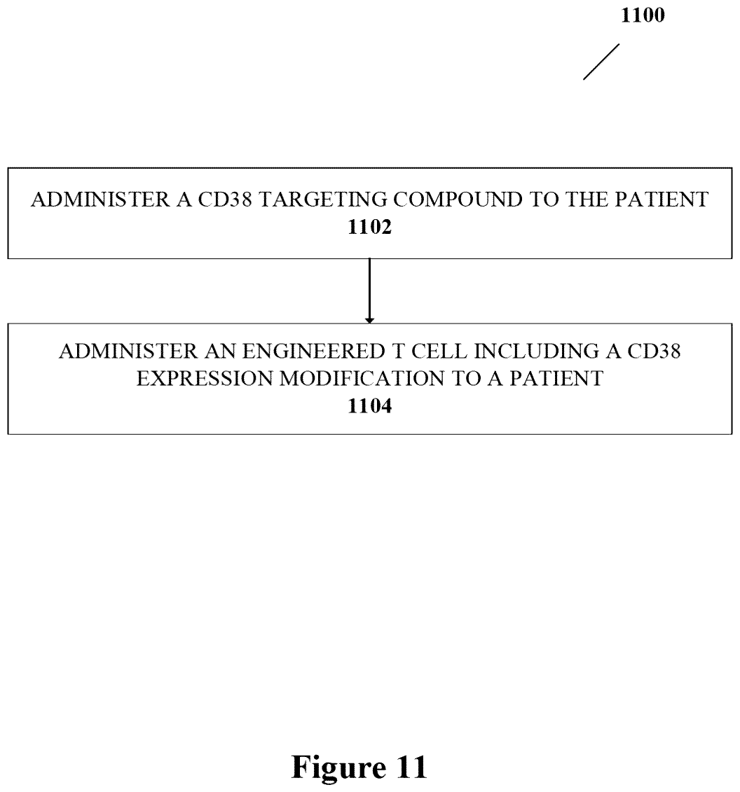

FIG. 11 illustrates a process for treating a patient using a T cell and a CD38 targeting compound 1100 in accordance with various embodiments.

Step 1102 includes administering a CD38 targeting compound to the patient.

Step 1104 includes administering an engineered T cell including a CD38 expression modification to a patient.

In various embodiments, the engineered T cell may target include CD20, CD19, CLL-1, BCMA, EGFR, HER2, GPC3, GD2, or any combination thereof. In various embodiments, the engineered T cell may comprise an antigen binding molecule including a binding motif that binds an antigen selected from the group comprising 5T4, alphafetoprotein, B cell maturation antigen (BCMA), B cell receptor, CA-125, carcinoembryonic antigen, CD19, CD20, CD22, CD23, CD30, CD33, CD40, CD56, CD79, CD78, CD123, CD138, c-Met, CSPG4, IgM, C-type lectin-like molecule 1 (CLL-1), EGFRvIII, epithelial tumor antigen, ERBB2, FLT3, folate binding protein, GD2, GD3, HER1-HER2 in combination, HER2-HER3 in combination, HER2/Neu, HERV-K, HIV-1 envelope glycoprotein gp41, HIV-1 envelope glycoprotein gpl20, IL-IIRalpha, kappa chain, lambda chain, melanoma-associated antigen, mesothelin, MUC-1, mutated p53, mutated ras, prostate-specific antigen, ROR1, VEGFR2, EphA3 (EPH receptor A3), BAFFR (B-cell activating factor receptor), and combinations thereof.

In various embodiments, the method may include administering a CD38 targeting compound to the patient followed by administering an engineered T cell including a CD38 expression modification and then administering a CD38 targeting compound.

In various embodiments, the CD38 targeting compound comprises an antigen binding molecule having specificity for a CD38 antigen. In various embodiments, the antigen binding molecule includes anti-CD38 antibodies. In various embodiments, the antigen binding molecule includes a DARPin. In various embodiments, the antigen binding molecule includes a nano-body. In various embodiments, the antigen binding molecule includes a ligand trap. In various embodiments, the antigen binding molecule includes a synthetic component. In various embodiments, the CD38 targeting compound comprises isatuximab. In various embodiments, the CD38 targeting compound comprises daratumumab. In various embodiments, the CD38 targeting compound comprises MOR202. In various embodiments, the CD38 targeting compound comprises TAK079.

In various embodiments, the CD38 expression modification comprises a CD38 gene knock-out. In various embodiments, the gene knock-out is Cas9 mediated. In various embodiments, the Cas9 mediated gene knock-out uses a complex including a Cas9 molecule and a guide nucleotide sequence. In various embodiments, the nucleotide guide sequence comprising SEQ ID NO: 1. In various embodiments, the nucleotide guide sequence comprising SEQ ID NO: 2. In various embodiments, the nucleotide guide sequence comprising SEQ ID NO: 3. In various embodiments, the nucleotide guide sequence comprising SEQ ID NO: 4. In various embodiments, the gene knock-out is Cas12 mediated.

In various embodiments, the gene knock-out is base editor mediated. In various embodiments, the base editor includes a cytosine base editor (CBE). In various embodiments, the cytosine base editor includes a cytidine base editor comprised of a Cas9 nickase fused to a cytidine deaminase derived from APOBEC1. In various embodiments, the base editor includes an adenine base editor (ABE). In various embodiments, the base editor is complexed with a nucleotide guide sequence. In various embodiments, the nucleotide guide sequence comprising SEQ ID NO: 5. In various embodiments, the nucleotide guide sequence comprising SEQ ID NO: 6. In various embodiments, the nucleotide guide sequence comprising SEQ ID NO: 7. In various embodiments, the nucleotide guide sequence comprising SEQ ID NO: 8. In various embodiments, the nucleotide guide sequence comprising SEQ ID NO: 9. In various embodiments, the nucleotide guide sequence comprising SEQ ID NO: 10.

In various embodiments, a CD38 gene knock-in. In various embodiments, the CD38 gene knock-in uses homology directed repair.

In various embodiments, the CD38 expression modification uses RNAi. In various embodiments, the CD38 expression modification uses anti-sense oligonucleotide (ASO). In various embodiments, the CD38 expression modification uses epigenetic editing. In various embodiments, the epigenetic editing uses a TALEN. In various embodiments, the epigenetic editing uses a zinc finger nuclease. In various embodiments, the epigenetic editing uses CRISPRi.

In various embodiments, the CD38 expression modification comprises a single amino acid substitution. In various embodiments, the single amino acid substitution occurs at an epitope specific for Daratumumab. In various embodiments, the single amino acid substitution occurs at an epitope specific for Isatuximab. In various embodiments, the single amino acid substitution is mediated by a base editor. In various embodiments, the substitution replaces serine 274. In various embodiments, the substitution replaces the serine with a phenylalanine. In various embodiments, the base editor includes a nucleotide guide sequence comprising SEQ ID NO: 11.

In various embodiments, the substitution replaces threonine 116. In various embodiments, the substitution replaces the threonine with an alanine. In various embodiments, the base editor includes a nucleotide guide sequence comprising SEQ ID NO: 16. In various embodiments, the base editor includes a nucleotide guide sequence comprising SEQ ID NO: 17. In various embodiments, the base editor includes a nucleotide guide sequence comprising SEQ ID NO: 18. In various embodiments, the base editor includes a nucleotide guide sequence comprising SEQ ID NO: 19.

In various embodiments, the engineered T cell expresses a CD38 antigen nucleotide encoding sequence comprising SEQ ID NO: 21. In various embodiments, the engineered T cell expresses a CD38 antigen protein molecule comprising SEQ ID NO: 23. In various embodiments, the engineered T cell expresses a CD38 antigen protein molecule comprising SEQ ID NO: 24.

In various embodiments, the engineered T cell includes a CAR T cell. In various embodiments, the system is an allogeneic therapeutic. In various embodiments, the system is an autologous therapeutic.

In various embodiments, the engineered T cell targets CD20. In various embodiments, the engineered T cell targets CD19. In various embodiments, the engineered T cell targets CLL-1. In various embodiments, the engineered T cell targets BCMA. In various embodiments, the engineered T cell targets EGFR. In various embodiments, the CAR T cells target HER2. In various embodiments, the engineered T cell targets GPC3. In various embodiments, the engineered T cell targets GD2.

### VIII. Exemplary Kits for A Combination Therapy Using A T Cell and A Cd38 Targeting Compound

In various embodiments, the CD38 targeting compound comprises an antigen binding molecule having specificity for a CD38 antigen. In various embodiments, the antigen binding molecule includes anti-CD38 antibodies. In various embodiments, the antigen binding molecule includes a DARPin. In various embodiments, the antigen binding molecule includes a nano-body. In various embodiments, the antigen binding molecule includes a ligand trap. In various embodiments, the antigen binding molecule includes a synthetic component. In various embodiments, the CD38 targeting compound comprises isatuximab. In various embodiments, the CD38 targeting compound comprises daratumumab. In various embodiments, the CD38 targeting compound comprises MOR202. In various embodiments, the CD38 targeting compound comprises TAK079.

In various embodiments, the CD38 expression modification comprises a CD38 gene knock-out. In various embodiments, the gene knock-out is Cas9 mediated. In various embodiments, the Cas9 mediated gene knock-out uses a complex including a Cas9 molecule and a guide nucleotide sequence. In various embodiments, the nucleotide guide sequence comprising SEQ ID NO: 1. In various embodiments, the nucleotide guide sequence comprising SEQ ID NO: 2. In various embodiments, the nucleotide guide sequence comprising SEQ ID NO: 3. In various embodiments, the nucleotide guide sequence comprising SEQ ID NO: 4. In various embodiments, the gene knock-out is Cas12 mediated.

In various embodiments, the gene knock-out is base editor mediated. In various embodiments, the base editor includes a cytosine base editor (CBE). In various embodiments, the cytosine base editor includes a cytidine base editor comprised of a Cas9 nickase fused to a cytidine deaminase derived from APOBEC1. In various embodiments, the base editor includes an adenine base editor (ABE). In various embodiments, the base editor is complexed with a nucleotide guide sequence. In various embodiments, the nucleotide guide sequence comprising SEQ ID NO: 5. In various embodiments, the nucleotide guide sequence comprising SEQ ID NO: 6. In various embodiments, the nucleotide guide sequence comprising SEQ ID NO: 7. In various embodiments, the nucleotide guide sequence comprising SEQ ID NO: 8. In various embodiments, the nucleotide guide sequence comprising SEQ ID NO: 9. In various embodiments, the nucleotide guide sequence comprising SEQ ID NO: 10.

In various embodiments, a CD38 gene knock-in is described. In various embodiments, the CD38 gene knock-in uses homology directed repair.

In various embodiments, the CD38 expression modification uses RNAi. In various embodiments, the CD38 expression modification uses anti-sense oligonucleotide (ASO). In various embodiments, the CD38 expression modification uses epigenetic editing. In various embodiments, the epigenetic editing uses a TALEN. In various embodiments, the epigenetic editing uses a zinc finger nuclease. In various embodiments, the epigenetic editing uses CRISPRi.

In various embodiments, the CD38 expression modification comprises a single amino acid substitution. In various embodiments, the single amino acid substitution occurs at an epitope specific for Daratumumab. In various embodiments, the single amino acid substitution occurs at an epitope specific for Isatuximab. In various embodiments, the single amino acid substitution is mediated by a base editor. In various embodiments, the substitution replaces serine 274. In various embodiments, the substitution replaces the serine with a phenylalanine. In various embodiments, the base editor includes a nucleotide guide sequence comprising SEQ ID NO: 11.

In various embodiments, the substitution replaces threonine 116. In various embodiments, the substitution replaces the threonine with an alanine. In various embodiments, the base editor includes a nucleotide guide sequence comprising SEQ ID NO: 11. In various embodiments, the base editor includes a nucleotide guide sequence comprising SEQ ID NO: 16. In various embodiments, the base editor includes a nucleotide guide sequence comprising SEQ ID NO: 17. In various embodiments, the base editor includes a nucleotide guide sequence comprising SEQ ID NO: 18. In various embodiments, the base editor includes a nucleotide guide sequence comprising SEQ ID NO: 19.

In various embodiments, the engineered T cell expresses a CD38 antigen nucleotide encoding sequence comprising SEQ ID NO: 21. In various embodiments, the engineered T cell expresses a CD38 antigen protein molecule comprising SEQ ID NO: 23. In various embodiments, the engineered T cell expresses a CD38 antigen protein molecule comprising SEQ ID NO: 24.

In various embodiments, the engineered T cell includes a CAR T cell. In various embodiments, the system is an allogeneic therapeutic. In various embodiments, the system is an autologous therapeutic.

In various embodiments, the engineered T cell targets CD20. In various embodiments, the engineered T cell targets CD19. In various embodiments, the engineered T cell targets CLL-1. In various embodiments, the engineered T cell targets BCMA. In various embodiments, the engineered T cell targets EGFR. In various embodiments, the CAR T cells target HER2. In various embodiments, the engineered T cell targets GPC3. In various embodiments, the engineered T cell targets GD2. In various embodiments, the engineered T cell may target include CD20, CD19, CLL-1, BCMA, EGFR, HER2, GPC3, GD2, or any combination thereof. In various embodiments, the engineered T cell may comprise an antigen binding molecule including a binding motif that binds an antigen selected from the group comprising 5T4, alphafetoprotein, B cell maturation antigen (BCMA), B cell receptor, CA-125, carcinoembryonic antigen, CD19, CD20, CD22, CD23, CD30, CD33, CD40, CD56, CD79, CD78, CD123, CD138, c-Met, CSPG4, IgM, C-type lectin-like molecule 1 (CLL-1), EGFRvIII, epithelial tumor antigen, ERBB2, FLT3, folate binding protein, GD2, GD3, HER1-HER2 in combination, HER2-HER3 in combination, HER2/Neu, HERV-K, HIV-1 envelope glycoprotein gp41, HIV-1 envelope glycoprotein gpl20, IL-IIRalpha, kappa chain, lambda chain, melanoma-associated antigen, mesothelin, MUC-1, mutated p53, mutated ras, prostate-specific antigen, ROR1, VEGFR2, EphA3 (EPH receptor A3), BAFFR (B-cell activating factor receptor), and combinations thereof.

### IX. Examples

Table 4 shows a nucleotide encoding sequence for a CD38 antigen.

TABLE 4
CD38 Antigen Encoding Sequence

| | | SEQ ID |
|---|---|---|
| CD38 | NUCLEOTIDE ENCODING SEQUENCE | NO. |
| Antigen | ATGGCCAACTGCGAGTTCAGCCCGGT | 21 |
| Coding | GTCCGGGGACAAACCCTGCTGCCGGCT | |
| Sequence | CTCTAGGAGAGCCCAACTCTGTCTTG | |
| (CDS) | GCGTCAGTATCCTGGTCCTGATCCTC | |
| | GTCGTGGTGCTCGCGGTGGTCGTCCC | |
| | GAGGTGGCGCCAGCAGTGGAGCGGTC | |
| | CGGGCACCACCAAGCGCTTTCCCGAG | |
| | ACCGTCCTGGCGCGATGCGTCAAGTA | |
| | CACTGAAATTCATCCTGAGATGAGAC | |
| | ATGTAGACTGCCAAAGTGTATGGGAT | |
| | GCTTTCAAGGGTGCATTTATTTCAAA | |
| | ACATCCTTGCAACATTACTGAAGAAG | |
| | ACTATCAGCCACTAATGAAGTTGGGA | |
| | ACTCAGACCGTACCTTGCAACAAGAT | |
| | TCTTCTTTGGAGCAGAATAAAAGATC | |
| | TGGCCCATCAGTTCACACAGGTCCAG | |
| | CGGGACATGTTCACCCTGGAGGACAC | |
| | GCTGCTAGGCTACCTTGCTGATGACC | |
| | TCACATGGTGTGGTGAATTCAACACT | |
| | TCCAAAATAAACTATCAATCTTGCCC | |
| | AGACTGGAGAAAGGACTGCAGCAACA | |
| | ACCCTGTTTCAGTATTCTGGAAAACG | |
| | GTTTCCCGCAGGTTTGCAGAAGCTGC | |
| | CTGTGATGTGGTCCATGTGATGCTCA | |
| | ATGGATCCCGCAGTAAAATCTTTGAC | |
| | AAAAACAGCACTTTTGGGAGTGTGGA | |
| | AGTCCATAATTTGCAACCAGAGAAGG | |
| | TTCAGACACTAGAGGCCTGGGTGATA | |
| | CATGGTGGAAGAGAAGATTCCAGAGA | |
| | CTTATGCCAGGATCCCACCATAAAAG | |
| | AGCTGGAATCGATTATAAGCAAAAGG | |
| | AATATTCAATTTTCCTGCAAGAATAT | |
| | CTACAGACCTGACAAGTTTCTTCAGT | |
| | GTGTGAAAAATCCTGAGGATTCATCT | |
| | TGCACATCTGAGATCTGA | |

Table 5 shows exemplary CD38 antigen amino acid sequences.

TABLE 5
CD38 Antigen Amino Acid Sequences

| CD38 | | SEQ ID |
|---|---|---|
| Antigen | AMINO ACID SEQUENCE | NO. |
| CD38 | MANCEFSPVSGDKPCCRLSR | 22 |
| protein | RAQLCLGVSILVLILVVVLA | |
| | VVVPRWRQQWSGPGTTKRFP | |
| | ETVLARCVKYTEIHPEMRHV | |
| | DCQSVWDAFKGAFISKHPCN | |
| | ITEEDYQPLMKLGTQTVPCN | |
| | KILLWSRIKDLAHQFTQVQR | |
| | DMFTLEDTLLGYLADDLTWC | |
| | GEFNTSKINYQSCPDWRKDC | |
| | SNNPVSVFWKTVSRRFAEAA | |
| | CDVVHVMLNGSRSKIFDKNS | |
| | TFGSVEVHNLQPEKVQTLEA | |
| | WVIHGGREDSRDLCQDPTIK | |
| | ELESIISKRNIQFSCKNIYR | |
| | PDKFLQCVKNPEDSSCTSEI | |
| CD38 | MANCEFSPVSGDKPCCRLSR | 23 |
| S274F | RAQLCLGVSILVLILVVVLA | |
| | VVVPRWRQQWSGPGTTKRFP | |
| | ETVLARCVKYTEIHPEMRHV | |
| | DCQSVWDAFKGAFISKHPCN | |
| | ITEEDYQPLMKLGTQTVPCN | |
| | KILLWSRIKDLAHQFTQVQR | |
| | DMFTLEDTLLGYLADDLTWC | |
| | GEFNTSKINYQSCPDWRKDC | |
| | SNNPVSVFWKTVSRRFAEAA | |
| | CDVVHVMLNGSRSKIFDKNS | |
| | TFGSVEVHNLQPEKVQTLEA | |
| | WVIHGGREDSRDLCQDPTIK | |
| | ELESIISKRNIQFFCKNIYR | |
| | PDKFLQCVKNPEDSSCTSEI | |
| CD38 | MANCEFSPVSGDKPCCRLSR | 24 |
| T116A | RAQLCLGVSILVLILVVVLA | |
| | VVVPRWRQQWSGPGTTKRFP | |
| | ETVLARCVKYTEIHPEMRHV | |
| | DCQSVWDAFKGAFISKHPCN | |
| | ITEEDYQPLMKLGTQAVPCN | |
| | KILLWSRIKDLAHQFTQVQR | |
| | DMFTLEDTLLGYLADDLTWC | |
| | GEFNTSKINYQSCPDWRKDC | |
| | SNNPVSVFWKTVSRRFAEAA | |
| | CDVVHVMLNGSRSKIFDKNS | |
| | TFGSVEVHNLQPEKVQTLEA | |
| | WVIHGGREDSRDLCQDPTIK | |
| | ELESIISKRNIQFSCKNIYR | |
| | PDKFLQCVKNPEDSSCTSEI | |

### Example 1: Deletion of CD38 in Non-Transduced Human Primary T Cells Using CRISPR/Cas9

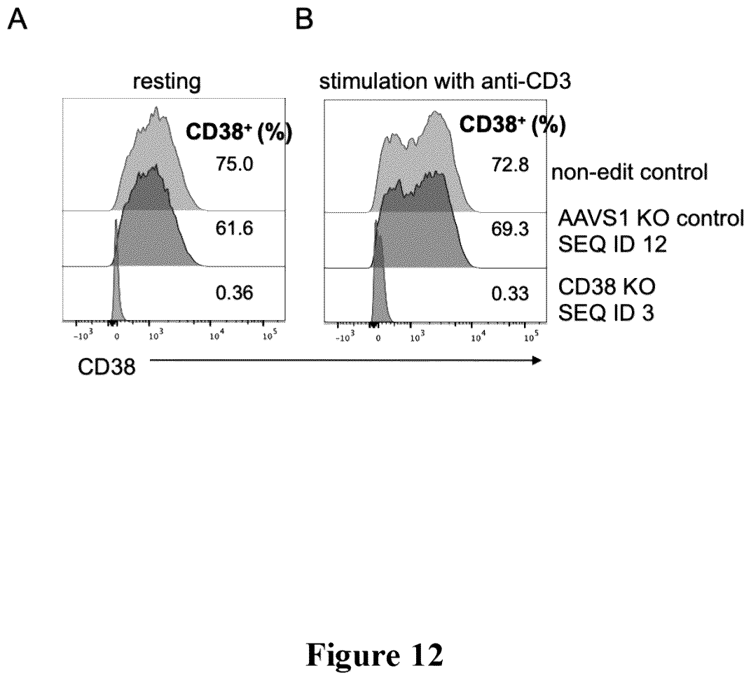

FIG. 12 is experimental data for deletion of CD38 in non-transduced human primary T cells using CRISPR/Cas9 with sgRNA SEQ ID NO. 3. Flow cytometry was performed 7 days after T cell activation. Resting T cells were stimulated once on day 0 with plate-bound OKT3 antibodies. Stimulated T cells were exposed on day 0 and day 6 to plate-bound OKT3 antibodies. Guides targeting safe harbor locus adeno-associated virus integration site 1 (AAVS1) were included to control for the effect of gene editing. This example demonstrates efficient knockout of CD38 with CRISPR/Cas9. FIG. 12, panel A is flow cytometric analysis data for resting T cells. FIG. 12, panel B, is flow cytometric analysis data for T cells stimulated with plate-bound anti-CD3 antibodies. CD38 expression was detected with the monoclonal antibody clone HIT2 obtained from Biolegend™

### CD4 and CD8 T Cell Isolation

CD4 and CD8 T cells were isolated from healthy donor PBMC with CD4 and CD8 CliniMACS® beads and CliniMACS® instrument. Cells were frozen at 25×10⁶ cells/mL and frozen down in CryoStor® cell cryopreservation media (sourced from Sigma Aldrich®) and stored under liquid nitrogen.

### T Cell Culture and Activation

Healthy donor T cells were activated with plate-bound MACS GMP CD3 Pure (1.23 μg/mL for coating) and soluble mouse anti-human CD28 antibody (1 μg/mL final concentration) at 1×10⁶ cells/mL on day 0 in CTS™ OpTmizer™ media supplemented with CTS™ OpTmizer™ Cell SR, CTS™ OpTmizer™ T cell expansion supplement, Pen/Strep/Glutamine, and 300 IU/mL IL-2.

### Cas9 Gene Editing

72 hours after activation, T cells were electroporated using the Lonza™ 4D Nucleofector® X unit and the Lonza™ P3 Primary Cell and the P3 Primary Cell 4D-Nucleofector™ X Kit according to the manufacturer's instructions. Ribonucleoprotein was prepared by combining 5 μg TrueCut™ Cas9 Protein v2 (Thermo Fisher Scientific™) with 5 μg sgRNA with 2′-O-methyl phosphorothioate modification (sourced from Synthego™). The spacer sequences are provided in Table 6. sgRNA sequences are provided in Table 6 SEQ ID NOS. 5, 6, 7, 8, and 16. Ribonucleoprotein and 1×10⁶ T cells were combined and electroporated with pulse code EO115. T cells were transferred to media as described above supplemented with 5% human AB serum (Valley Biomedical™).

TABLE 6
Nucleotide Guide Sequences
for Targeting Enzymes

| | | | SEQ |
|---|---|---|---|
| | GUIDE NUCLEOTIDE | | ID |
| TARGET | SEQUENCE | EFFECTOR | NO. |
| CD38 KO | AUGUAGACUGCCAAAGUGUA | Cas9 | 1 |
| CD38 KO | UGUAGACUGCCAAAGUGUAU | Cas9 | 2 |
| CD38 KO | AGUGUAUGGGAUGCUUUCAA | Cas9 | 3 |
| CD38 KO | UCUUCUUCAGUAAUGUUGCA | Cas9 | 4 |
| CD38 KO | CTATCAGCCACTAATGAAGT | CBE | 5 |
| CD38 KO | TATCAGCCACTAATGAAGTT | CBE | 6 |
| CD38 KO | AATTACCTTGTTGCAAGGTA | CBE | 7 |
| CD38 KO | CTCCACTGCTGGCGCCACCT | CBE | 8 |
| CD38 KO | GCGCCAGCAGTGGAGCGGTC | CBE | 9 |
| CD38 KO | CGCCAGCAGTGGAGCGGTCC | CBE | 10 |
| CD38 S274F | TTTCCTGCAAGAATATCTAC | CBE | 11 |
| AAVS1 KO | GGGGCCACTAGGGACAGGAT | CAS9 | 12 |
| AAVS1 KO | CUCCAGGUUCUCAUCAAUGC | CAS9 | 13 |
| AAVS1 KO | GTCACCAATCCTGTCCCTAG | CBE | 14 |
| AAVS1 KO | ACAGCATGTTTGCTGCCTCC | CBE | 15 |
| CD38 G113K | TGAGTTCCCAACTTCATTAG | CBE | 16 |
| CD38 T116A | TCAGACCGTACCTTGCAACA | CBE | 17 |
| | | and | |
| | | ABE | |
| CD38 P118F | GTACCTTGCAACAAGGTAAT | CBE | 18 |
| CD38 P118L | TACCTTGCAACAAGGTAATT | CBE | 19 |
| CD38 KO | TTGGCCATAGGGCTCCAGGC | ABE | 20 |

### Example 2: Deletion of CD38 in Non-Transduced Human Primary T Cells Using a Cytidine Base Editor

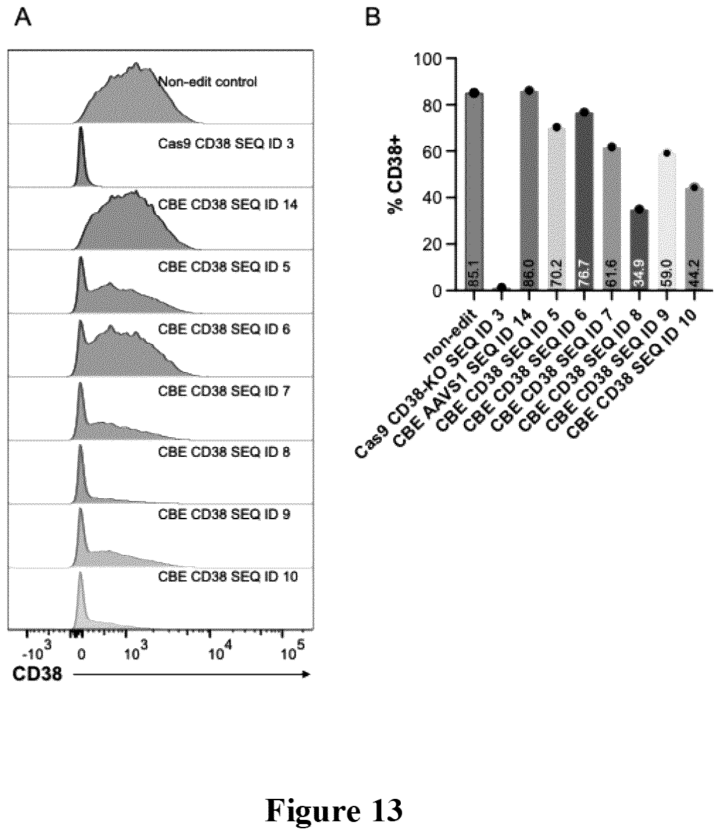

FIG. 13 is experimental data for deletion of CD38 in non-transduced human primary T cells using a cytidine base editor. CBE; Cytidine Base Editor. FIG. 13, panel A CD38 expression assessed by flow cytometry. FIG. 13, panel B Quantification of CD38+ T cells as shown in FIG. 13, panel A. Flow cytometry was performed 7 days after T cell activation. Cas9 gene editing was included as a positive control. This example demonstrates efficient loss of CD38 using a base editor.

T cell isolation, cell culturing, and flow cytometry were performed as described in example 1. Base editing was performed with sgRNA sequences provided in Table 6, SEQ ID NOS. 5, 6, 7, 8, 9, 10.

### Base Editing

Seventy-Two hours after activation, T cells were electroporated using a Lonza™ 4D Nucleofector® X unit and a Lonza P3 Primary Cell and a P3 Primary Cell 4D-Nucleofector™ X Kit according to the manufacturer's instructions. mRNA encoding a cytidine base editor that is comprised of a Cas9 nickase fused with an APOBEC1 deaminase domain. The mRNA encoding the base editor was synthesized with N1-Methyl-Pseudo-U modification sourced from TriLink Biotechnologies, Inc. 1×10⁶ T cells, 1 μg of mRNA, and 5 μg of sgRNA were combined and electroporated with pulse code EO115. sgRNA sequences are provided in Table 6. T cells were transferred to media as described above supplemented with 5% human AB serum sourced from Valley Biomedical, Inc.

### Example 3: Disruption of Daratumumab Binding to Non-Transduced Human Primary T Cells Using a Cytidine Base Editor and Cas9 Knock-Out

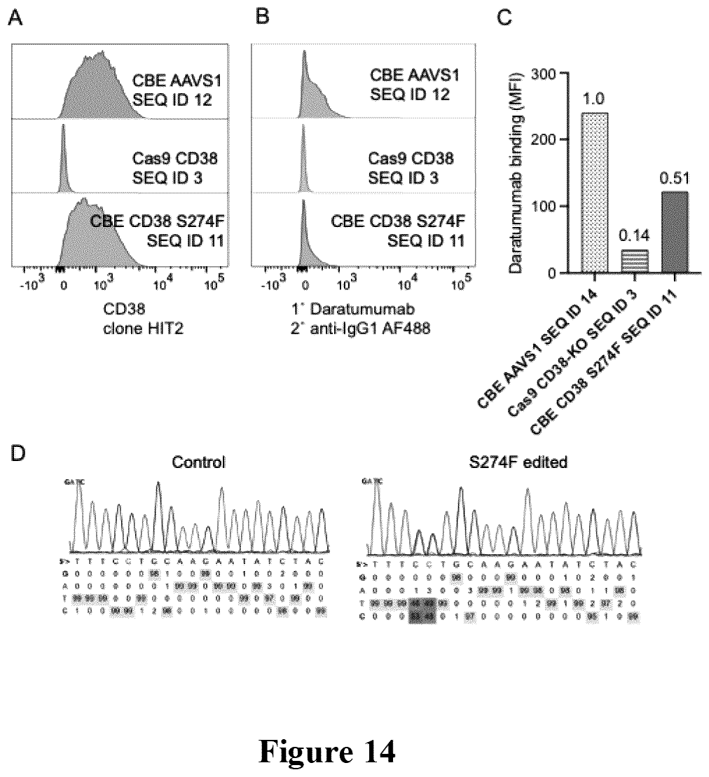

FIG. 14 is experimental data for disruption of Daratumumab binding to non-transduced human primary T cells using a cytidine base editor and Cas9 knock-out by installing a point mutation to replace serine 274 with phenylalanine (S274F) with a cytidine base editor and an sgRNA, SEQ ID 11. FIG. 14, panel A is flow cytometry data showing CD38 expression detected by anti-CD38 clone HIT2. The serine 274 phenylalanine mutation does not impact CD38 expression level. FIG. 14, panel B is flow cytometry data showing Daratumumab binding to CAR-T cells is diminished by the S274F mutation. Daratumumab binding is also diminished by knocking out CD38. FIG. 14, panel C is a bar graph of the data in FIG. 14, panel B. MFI; Mean florescence intensity. The corresponding numerical values are shown in Table 7. FIG. 14, panel D shows Sanger sequencing analysis to determine base editing efficiency.

TABLE 7
Daratumumab Binding Data

| | Cas9 CD38-KO | CBE CD38 S274F | CBE AAVS1 |
|---|---|---|---|
| Sample | SEQ ID 3 | SEQ ID 11 | SEQ ID 14 |
| MFI | 35.2 | 122 | 240 |

A point mutation was installed to replace serine 274 with phenylalanine SEQ ID NO. 11. Staining with anti-CD38 antibodies shows introducing the S274 mutation into T cells does not impact abundance of CD38 on the cell surface but abrogates binding by daratumumab. Installation of the S274 mutation was confirmed by Sanger sequencing. As expected, cells with Cas9 gene editing of CD38 also demonstrated loss of daratumumab binding. These results indicated deletion of CD38 with Cas9 or disruption of the daratumumab epitope with a base editor results in a loss of daratumumab binding, which therefore enables the edited T cells to escape daratumumab-mediated killing. The S274 mutation has been previously described to prevent binding by daratumumab demonstrated using recombinant CD38 expression. See de Weers et al. 2011 J. Immunol, PMID: 21187443.

T cell isolation, cell culturing, Cas9 gene editing and base editing were performed as described in Examples 1 and 2 using sgRNA sequences found in Table 6, SEQ ID NOS. 14, 3, and 11.

Analysis of editing by Polymerase Chain Reaction (PCR) and Sanger Sequencing

Cell pellets were collected 4 days after electroporation and treated with QuickExtract™ DNA Extraction Solution sourced from Biosearch Technologies, Inc. The protocol was in accordance to the manufacture's instructions. Polymerase chain reaction (PCR) was performed with Phusion® Hot Start Flex™ 2X Master Mix and DNA primers flanking the target region sourced from Integrated DNA Technologies, Inc. Sanger sequencing of PCR products was analyzed using EditR (https://moriaritylab.shinyapps.io/editr_v10/) or Beat (https://hanlab.cc/beat/).

### Flow Cytometry

Four days after electroporation, T cells were collected into 96 well plates and incubated with Becton Dickinson™ Pharmingen™ Human BD Fc Block and LIVE/DEAD™ Fixable Far Red Dead Cell Stain Kit sourced from Thermo Fisher Scientific, Inc. The protocol was in accordance to the manufacturer's instructions. Primary antibody staining with Daratumumab was performed in Becton Dickinson™ Stain Buffer for 30 minutes at 4° C. Cells were washed in twice with Becton Dickinson™ stain buffer and then stained with a secondary antibody specific for human IgG1 for 30 minutes, washed, and resuspended in Becton Dickinson™ stain buffer prior to acquisition. Flow cytometry data was collected on Becton Dickinson FACSymphony™ A5 Cell Analyzer™ with Becton Dickinson™ FACS DIVA™™M software and data was analyzed using FlowJo™ software sourced from Becton Dickinson Biosciences, Inc.

### Example 4: Deletion of CD38 in Chimeric Antigen Receptor (CAR) T Cells Targeting CD19 and CD20

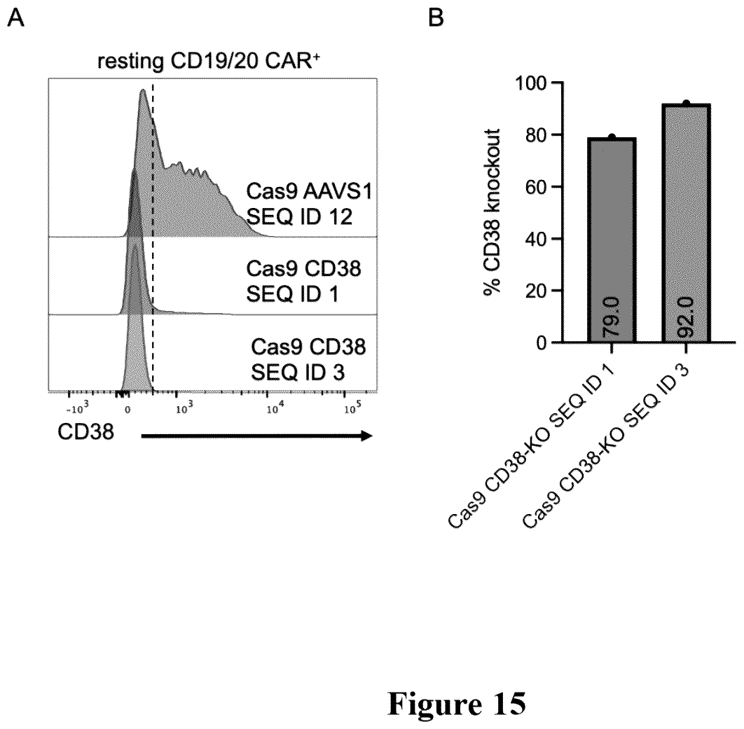

FIG. 15 is experimental data for deletion of CD38 in chimeric antigen receptor (CAR) T cells targeting CD19 and CD20. Details of the CD19 and CD20 dual-targeting CAR are disclosed in WO2020/123691, which is incorporated by reference in its entirety. The safe harbor locus AAVSI was included to control for the effects of gene editing. This example demonstrates efficient knock-out of CD38 in CAR-T cells, confirmed by flow cytometry and sequencing. FIG. 15, panel A is flow cytometry data showing CD38 expression in CAR+ T cells expressing a bicistronic CD19 and CD20 CAR. FIG. 15, panel B is experimental data showing frequency of indels quantified by Sanger sequencing and Inference of CRISPR Editing (ICE). The data in FIG. 15, panel B is displayed in Table 8.

TABLE 8
Editing Confirmation Data

| | Cas9 CD38-KO | Cas9 CD38-KO |
|---|---|---|
| | SEQ ID NO. 1 | SEQ ID NO. 3 |
| % indel | 79 | 92 |

### CD4 and CD8 T Cell Isolation

CD4 and CD8 T cells were isolated from healthy donor PBMC with CD4 and CD8 CliniMACS® beads and CliniMACS® instrument. Cells were frozen at least at 25×10⁶ cells/mL in and frozen down in CryoStor® cell cryopreservation media (Sigma Aldrich®) and stored under liquid nitrogen.

### T Cell Culture and Activation

Healthy donor T cells were activated with plate-bound MACS GMP CD3 Pure (1.23 μg/mL for coating) and soluble mouse anti-human CD28 antibody (1 μg/mL final concentration) at 1×10⁶ cells/mL on day 0 in CTS™ OpTmizer™ media supplemented with CTS™ OpTmizer™ Cell SR, CTS™ OpTmizer™ T cell expansion supplement, Pen/Strep/Glutamine, and 300 IU/mL IL-2.

### CAR Transduction

On day 1, cells were lentivirally transduced at an MOI of 5 with a bicistronic CD19-285 and CD20-41BBC, or not transduced. On day 3 virus was washed off, cells were counted, and prepared for electroporation.

### Cas9 Gene Editing

72 hours after activation, T cells were electroporated using the Lonza 4D Nucleofector® X unit and the Lonza™ P3 Primary Cell and the P3 Primary Cell 4D-Nucleofector™ X Kit according to the manufacturer's instructions. Ribonucleoprotein was prepared by combining 5 μg TrucCut™ Cas9 Protein v2 (sourced from Thermo Fisher Scientific, Inc.) with 5 μg sgRNA with 2′-O-methyl phosphorothioate modification (sourced from Synthego, Inc.). sgRNA sequences are provided in Table 6. Ribonucleoprotein and 1×10⁶ T cells were combined and electroporated with pulse code EO115. T cells were transferred to media as described above supplemented with 5% human AB serum (Valley Biomedical).

### Flow Cytometry

Four days after electroporation, T cells were collected into 96 well plates and incubated with Becton Dickinson™ Pharmingen™ Human Becton Dickinson™ Fc Block and LIVE/DEAD™ Fixable Far Red Dead Cell Stain Kit (sourced from Thermo Fisher Scientific, Inc.) according to manufacturer's instructions. Antibody staining with anti-idiotype antibodies was performed in Becton Dickinson™ Stain Buffer for 30 minutes at 4° C. Anti-idiotype antibodies were generated and conjugated in-house. Cells were washed in BD stain buffer and resuspended in Becton Dickinson™ Stain buffer for acquisition. Flow cytometry data was collected on Becton Dickinson™ FACSymphony™ A5 Cell Analyzer™ with Becton Dickinson™ FACS DIVAT software and data was analyzed using FlowJo™ software (sourced from Becton Dickinson Biosciences, Inc.).

Analysis of editing by Polymerase Chain Reaction (PCR) and Sanger Sequencing

Cell pellets were collected 4 days after electroporation and treated with QuickExtract™ DNA Extraction Solution (sourced from Biosearch Technologies, Inc.) according to the manufacturer's instructions. PCR was performed with Phusion® Hot Start Flex 2X Master Mix and DNA primers flanking the target region (sourced from Integrated DNA Technologies, Inc.). Sanger sequencing of PCR products was analyzed using ICE (https://ice.synthego.com/#/).

### Example 5: Loss of CD38 does not Impact CAR Expression

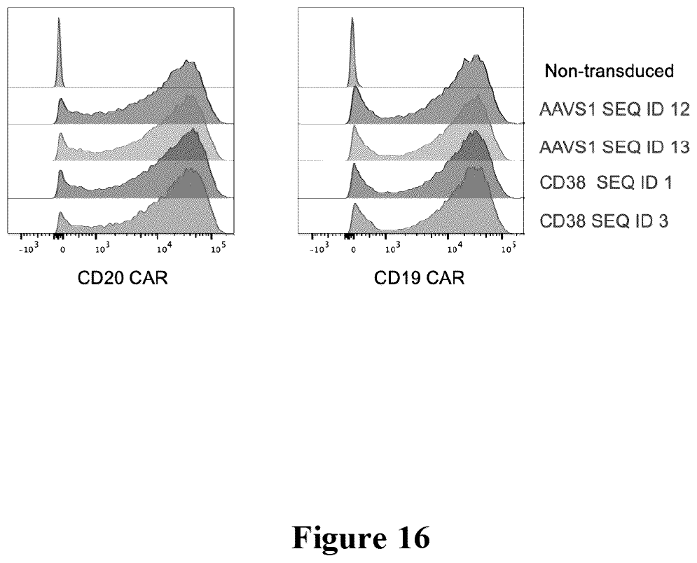

FIG. 16 is experimental flow cytometry data showing loss of CD38 does not impact CAR expression. CAR expression was assessed by flow cytometry 7 days after T cell activation. CAR-T cells were generated as described in example 4, and gene editing was confirmed in example 4. This example demonstrates the gene edited cells from example 4 express chimeric antigen receptors specific for CD20 and CD19.

### Example 6: Loss of CD38 does not Impact CAR-T Cell Expansion During Manufacturing

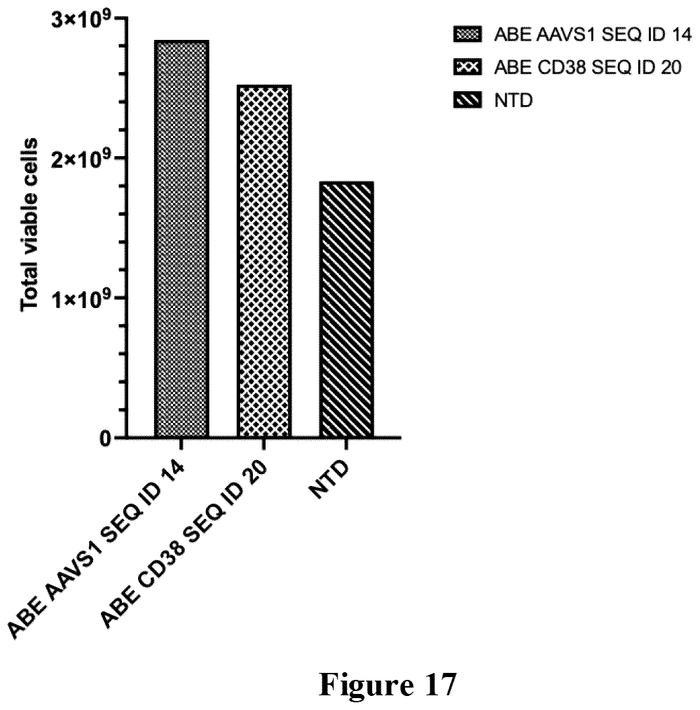

FIG. 17 is experimental data showing loss of CD38 does not impact CAR-T cell expansion during manufacturing. Lentiviral transduction of a CD19 and CD20 target CAR was performed 1 day following T cell activation. Gene editing with an adenine base editor (ABE) was performed 3 days post-activation with SEQ ID NOS. 14 and 20, and cell expansion was tracked until day 8. The associated data is shown in Table 9. NTD; non-transduced T cells that do not express a CAR and did not undergo gene editing.

TABLE 9
Expansion Data

| | ABE AAVS1 | ABE CD38 | |
|---|---|---|---|
| Sample | SEQ ID 14 | SEQ ID 20 | NTD |
| Expansion | 2.84E+09 | 2.53E+09 | 1.83E+09 |

### Example 7: CD38-Deficient CAR-T Cells Demonstrate Cytotoxicity

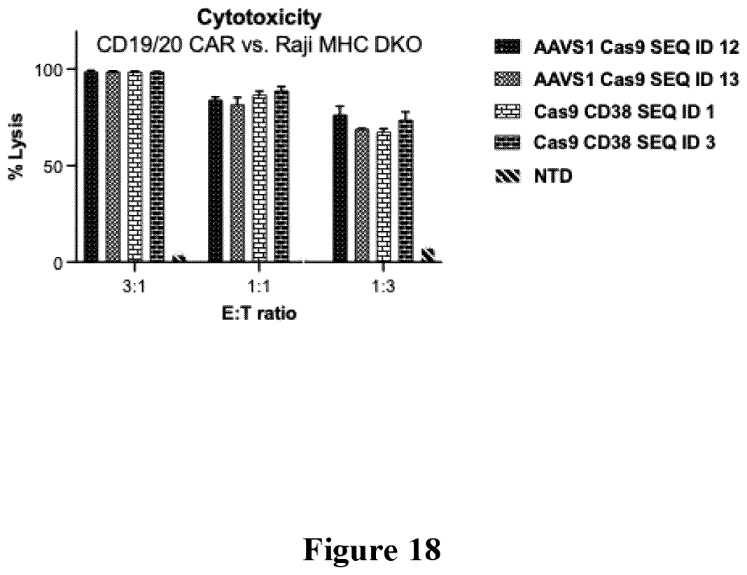

FIG. 18 is experimental data showing CD38-deficient CAR-T cells demonstrate cytotoxicity. CD19/20 CAR-T cells were co-cultured with Raji tumor cells at 1:3 effector to target cell ratio, 1:1 effector to target cell ratio, and 3:1 effector to target cell ratio. Raji cells were stably transduced with luciferase. Target cell death was assessed by luciferase activity. CAR T cell generation and gene editing was performed as described in example 4. This example demonstrates CAR-T cells deficient in CD38 maintain potent anti-tumor activity.

### Cytotoxicity Assay

CD19/20 CAR-T cells were co-cultured with Raji tumor cells at 1:3 effector to target cell ratio, 1:1 effector to target cell ratio, and 3:1 effector to target cell ratio (see FIG. 18). Raji cells were stably transduced with luciferase. Target cell death was assessed by luciferase activity. Briefly, the D-luciferin substrate was added to the co-culture wells at a final concentration of 0.14 mg/mL and plates were incubated at 37° C. in the dark for 10 minutes. Luminescent signal was read immediately after in a VarioSkan™ LUX or VarioSkan® Flash multimode microplate reader. T cell-mediated cytotoxicity was calculated as follows:

% Cytotoxicity=[1− luciferase signal of (sample of interest/target alone control)]*100. The data is shown in Tables 10A, 10B, and 10C.

TABLE 10A
Cytotoxicity (1:3 Effector to Target Cell)

| | | | |
|---|---|---|---|
| AAVS1 Cas9 SEQ ID | 80.52364842 | 77.2060495 | 72.35601674 |
| 12 | | | |
| AAVS1 Cas9 SEQ ID | 68.99830175 | 68.88998837 | 69.43957851 |
| 13 | | | |
| Cas9 CD38 SEQ ID 1 | 67.2853456 | 69.46364816 | 66.55924475 |
| Cas9 CD38 SEQ ID 3 | 70.63503737 | 72.81333993 | 78.37342712 |
| NTD | 5.173635719 | 7.993795381 | 9.437973871 |

TABLE 10B
Cytotoxicity (1:3 Effector to Target Cell)

| | | | |
|---|---|---|---|
| AAVS1 Cas9 SEQ ID | 85.49468359 | 82.69528276 | 84.61066228 |
| 12 | | | |
| AAVS1 Cas9 SEQ ID | 78.07995482 | 81.93281438 | 85.42469857 |
| 13 | | | |
| Cas9 CD38 SEQ ID 1 | 88.29776785 | 85.12634138 | 87.56108342 |
| Cas9 CD38 SEQ ID 3 | 91.2003045 | 87.31061071 | 88.72872824 |
| NTD | 1.133266213 | 1.125899369 | −2.030793409 |

TABLE 10A
Cytotoxicity (1:3 Effector to Target Cell)

| | | | |
|---|---|---|---|
| AAVS1 Cas9 SEQ ID | 98.19541414 | 98.75243218 | 99.38575407 |
| 12 | | | |
| AAVS1 Cas9 SEQ ID | 98.61890046 | 99.05383236 | 98.89359429 |
| 13 | | | |
| Cas9 CD38 SEQ ID 1 | 98.80202968 | 98.61890046 | 98.91648545 |
| Cas9 CD38 SEQ ID 3 | 98.58456373 | 98.43958643 | 98.71809546 |
| NTD | 5.375605662 | 2.979665026 | 3.406966541 |

### Example 8: CD38-Deficient CAR-T Cells Demonstrate Cytokine Release

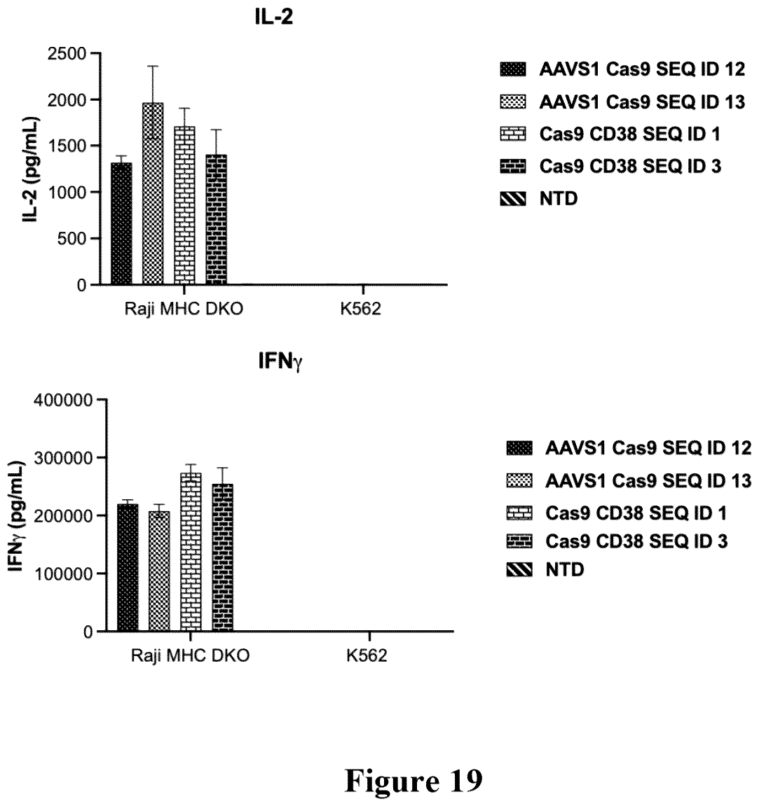

FIG. 19 is experimental data showing CD38-deficient CAR-T cells demonstrate cytokine release. CAR T cell generation and gene editing was performed as described in example 4. This example demonstrates CAR-T cells deficient in CD38 produce cytokines and function as expected.

### Cytokine Release Assay

CD19/20 CAR-T cells were co-culture with Raji tumor cells at 1:1 effector to target cell ratio. Cytokine abundance in cell culture supernatants was quantified by Meso Scale Discovery Electrochemiluminescence (MSD).

TABLE 11A
IFNG Abundance

| | | | |
|---|---|---|---|
| AAVS1 Cas9 | 220603.7643 | 227095.3111 | 212848.208 |
| SEQ ID 12 | | | |
| AAVS1 Cas9 | 202329.5715 | 200471.7165 | 221083.0268 |
| SEQ ID 13 | | | |
| Cas9 CD38 | 265354.675 | 290469.1652 | 265729.2839 |
| SEQ ID 1 | | | |
| Cas9 CD38 | 286792.1276 | 240866.6825 | 237191.0673 |
| SEQ ID 3 | | | |
| NTD | 206.1631213 | 228.4354552 | 250.7358431 |

TABLE 11B
IL-2 Abundance

| | | | |
|---|---|---|---|
| AAVS1 Cas9 | 1385.722957 | 1242.536504 | 1325.682119 |
| SEQ ID 12 | | | |
| AAVS1 Cas9 | 2085.626188 | 1530.219587 | 2289.011721 |
| SEQ ID 13 | | | |
| Cas9 CD38 | 1479.95457 | 1839.054726 | 1808.598877 |
| SEQ ID 1 | | | |
| Cas9 CD38 | 1680.827302 | 1149.320415 | 1387.017252 |
| SEQ ID 3 | | | |
| NTD | 6.574762203 | 5.222808282 | 4.328684755 |

### Example 9: Deletion of CD38 in CAR T Cells Targeting CD19, CLL1, and BCMA Using CRISPR/Cas9

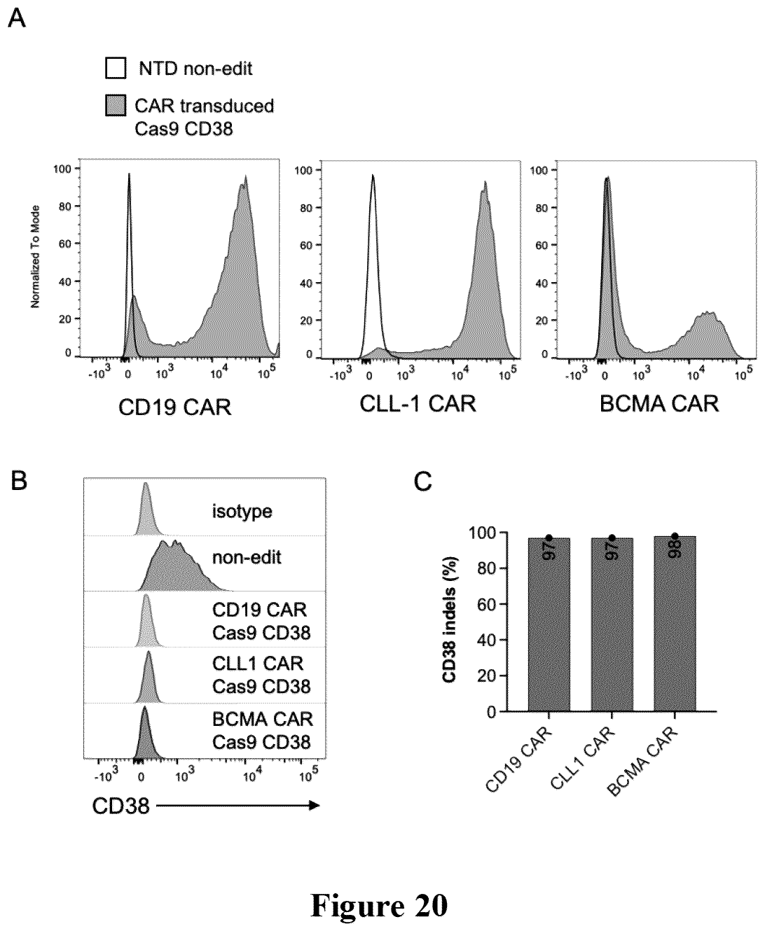

FIG. 20 is experimental data showing deletion of CD38 in CAR T cells targeting CD19, CLL1, and BCMA using CRISPR/Cas9. CAR-T cells were generated, and gene editing was assessed as described in Example 4. This example demonstrates that CD38 deletion is compatible with manufacturing CAR-T cells expressing various chimeric antigen receptors with specificity for different tumor antigens. FIG. 20, panel A is flow cytometry data showing CAR expression in transduced CAR T cells targeting CD19, CLL1, and BCMA versus non-transduced cells. FIG. 20, panel B is flow cytometry expression data for CAR T cells targeting CD19, CLL1, and BCMA, and non-transduced cells. FIG. 20, panel C is CD38 editing efficiency data assessed by Sanger sequencing and ICE. Table 12 shows the data from FIG. 20, panel C in numerical format.

TABLE 12
% indel for CD19, CLL1, and BCMA

| | CD19 CAR | CLL1 CAR | BCMA CAR |
|---|---|---|---|
| % indel | 97 | 97 | 98 |

### Example 10: Disruption of Isatuximab Binding to Non-Transduced Human Primary T Cells Using an Adenine Base Editor to Disrupt the CD38 Binding Epitope, or Cas9 to Knock-Out CD38

We discovered that that mutating the contacts between isatuximab and CD38 (SEQ ID NO. 21) resulted in loss of binding by Isatuximab. We examined the crystal structure of isatuximab (SAR650984) in complex with CD38 to nominate residues that are required for isatuximab binding (PDB ID code: 4CMH). Next, we designed base editing guides to mutate glycine 113, threonine 116, and phenylalanine 118 using cytidine or adenine editors (Table 6, SEQ ID NOS. 16, 17, 18 and 19). Base edited T cells were generated as described in Examples 1 and 2. The adenine base editor was comprised of a Cas9 nickase fused to a deoxyadenosine deaminase domain derived from TadA, and mRNA was synthesized as described in Example 3.

This example demonstrates the T116A mutation (SEQ ID NO. 17), but no other mutation, results in loss of isatuximab binding to T cells while maintaining cell surface expression of CD38. This mutation has not been previously reported. Together, these data indicated deletion of CD38 with Cas9 or disruption of the isatuximab epitope with a base editor results in a loss of isatuximab binding, which therefore enables the edited T cells to escape isatuximab-mediated killing.

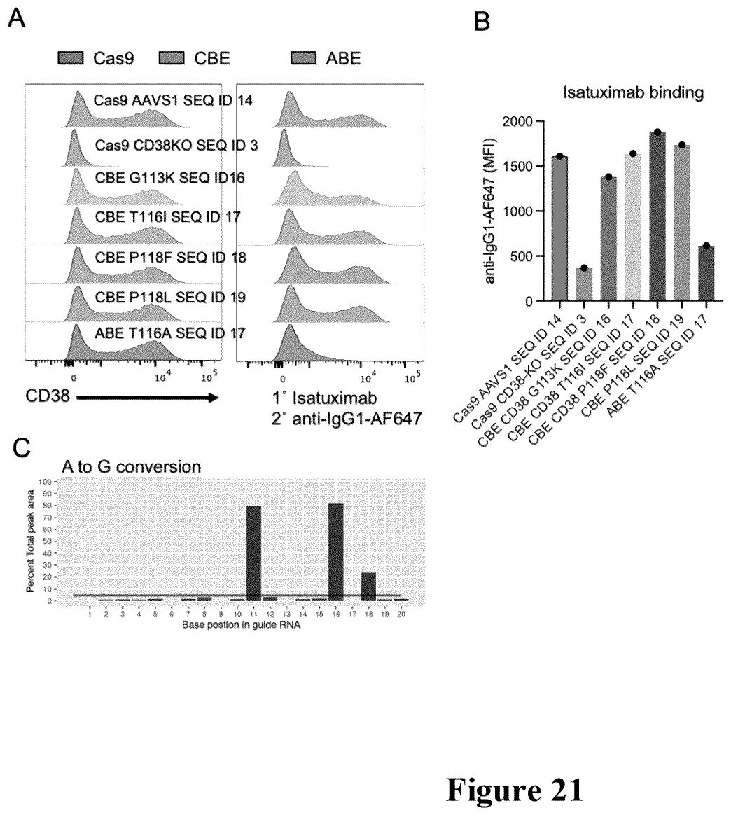

FIG. 21 is experimental data showing disruption of Isatuximab binding to non-transduced human primary T cells using an adenine base editor to disrupt the CD38 binding epitope, or Cas9 to knock-out CD38. FIG. 21, panel A is flow cytometry data showing CD38 expression (left), and flow cytometry data showing Isatuximab binding (right). FIG. 21, panel B is quantitative flow cytometry data showing Isatuximab binding. FIG. 21, panel C is data showing an analysis of Sanger sequencing to determine base editing efficiency using an adenine base editor to install a T116A point mutation into CD38.

TABLE 13
Isatuximab Binding

| sgRNA | Isatuximab binding MFI |
|---|---|
| Cas9 AAVS1 SEQ ID NO. 14 | 1611 |
| Cas9 CD38-KO SEQ ID NO. 3 | 369 |
| CBE CD38 G113K SEQ ID NO. 16 | 1383 |
| CBE CD38 T116I SEQ ID NO. 17 | 1640 |
| CBE CD38 P118F SEQ ID NO. 18 | 1881 |
| CBE CD38 P118L SEQ ID NO. 19 | 1737 |
| ABE CD38 T116A SEQ ID NO. 17 | 616 |

### Flow Cytometry

Four days after electroporation, T cells were collected into 96 well plates and incubated with BD Pharmingen™ Human BD Fc Block and LIVE/DEAD™ Fixable Far Red Dead Cell Stain Kit (sourced from Thermo Fisher Scientific, Inc.) according to manufacturer's instructions. Primary antibody staining with Isatuximab was performed in BD Stain Buffer for 30 minutes at 4° C. Cells were washed in twice with BD stain buffer and then stained with a secondary antibody specific for human IgG1 for 30 minutes, washed, and resuspended in BD stain buffer prior to acquisition. Flow cytometry data was collected on BD FACSymphony™ A5 Cell Analyzer with BD FACS DIVA™ software and data was analyzed using FlowJo™ software (sourced from BD Biosciences, Inc).

### Example 11: Disruption of Isatuximab Binding to Human Primary CAR-T Cells Using an Adenine Base Editor to Disrupt the CD38 Binding Epitope with T117A Point Mutation Preserves CD38 Catalytic Activity

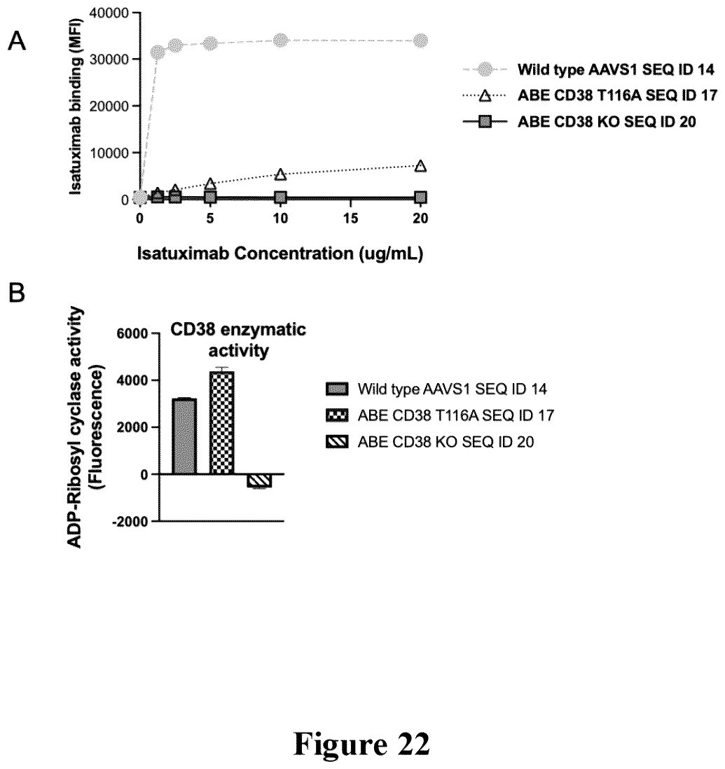

FIG. 22 is experimental data showing Isatuximab binding to CAR-T cells is diminished across a range of Isatuximab concentrations. FIG. 22, panel A shows Isatuximab binding assessed by flow cytometry. The CD38 T116A mutation reduces Isatuximab binding to CAR-T cells. FIG. 22, panel B shows ADP-ribosyl cyclase enzymatic activity measured in cell lysates using CD38 Activity Assay Kit (Abcam cat. ab284540). The CD38 T116A mutation preserves the catalytic activity of CD38, while CD38 knock out eliminates catalytic activity.

### Example 12: In Vivo Study Design to Model Combination Treatment with Anti-CD38 Antibodies and CAR-T Cells in NSG Mice

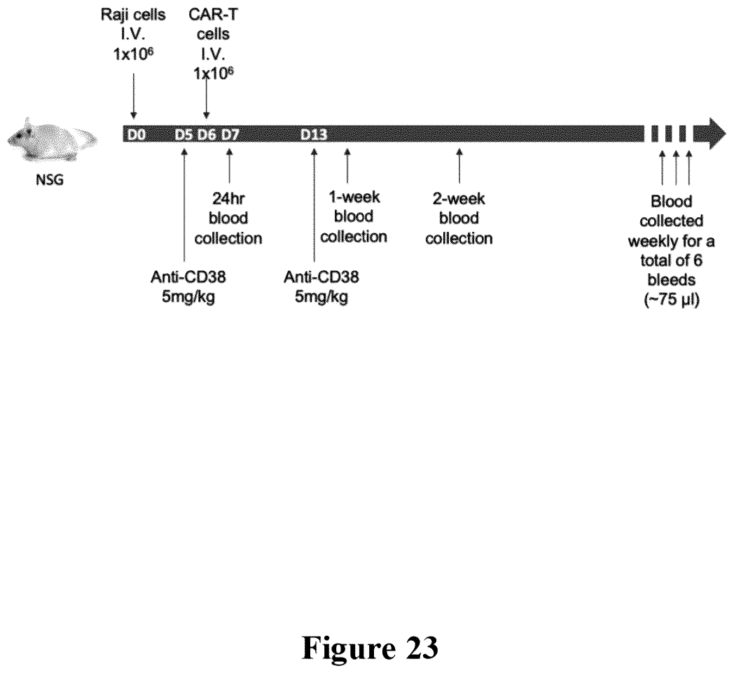

FIG. 23 is a schematic depicting an in vivo study of daratumumab and CAR-T cell combination therapy using a disseminated xenograft mouse model of human Burkitt's B-cell lymphoma. To establish the model, Raji Luc cells were first engineered to 1) stably express luc to facilitate noninvasive tumor measurement and 2) knock out MHC I/II expression (MHC I/II DKO) to reduce background alloreactivity (hereafter referred to as Raji Luc). NOD-scid IL2Rgamma^{null} (NSG) mice were implanted IV via the lateral tail vein with 1×10⁶ Raji tumor cells on day 0. Daratumumab was injected intraperitoneally (IP) on days 5 and 7 at 5 mg/kg. Dose levels of 5 or 1×10⁶ CAR-T cells were administered on day 7. Peripheral blood was sampled on day 7 and 14 and CAR-T abundance was assessed by flow cytometry. Tumor burden was monitored by bioluminescent imaging (IVIS Spectrum, Spectral Instruments Imaging). Mice were injected IP with luciferin prior to image collection to facilitate detection of tumor burden by luciferase activity.

### Example 13: CD3E/CD38 KO CAR T Cells Demonstrated Anti-Tumor Efficacy Against Raji Disseminated B-Cell Lymphoma Model in NSG Mice in the Absence or Presence of Anti-CD38 Ab

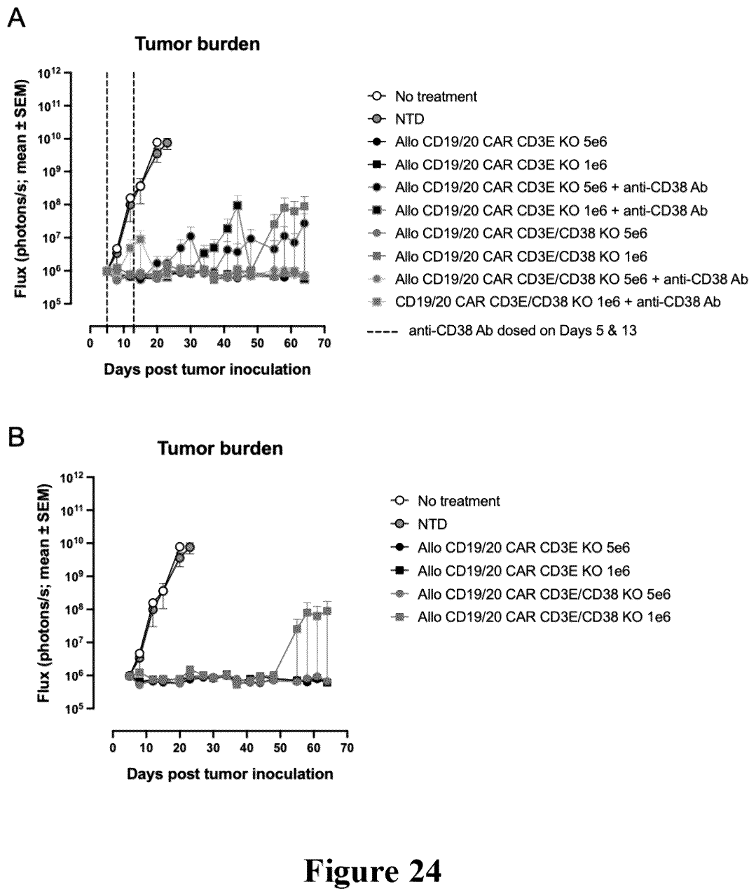

FIG. 24 is experimental data showing CD38 knock out CAR-T cells efficiently eliminate tumor cells in vivo with or without coadministration of daratumumab. CD19/20 targeting CAR-T cells were generated with adenine base editing. FIG. 24, panel A shows tumor burden assessed by bioluminescent imaging. Some groups received CAR-T treatment alone while some groups received CAR-T and Daratumumab combination. FIG. 24, panel B shows tumor burden in mice treated with control CAR-T cells (CD3E knockout) or CAR-T cells with both CD3E and CD38 knockout.

### Example 14: CD38 Knockout is Required to Prevent Daratumumab-Mediated Depletion of CAR-T Cells

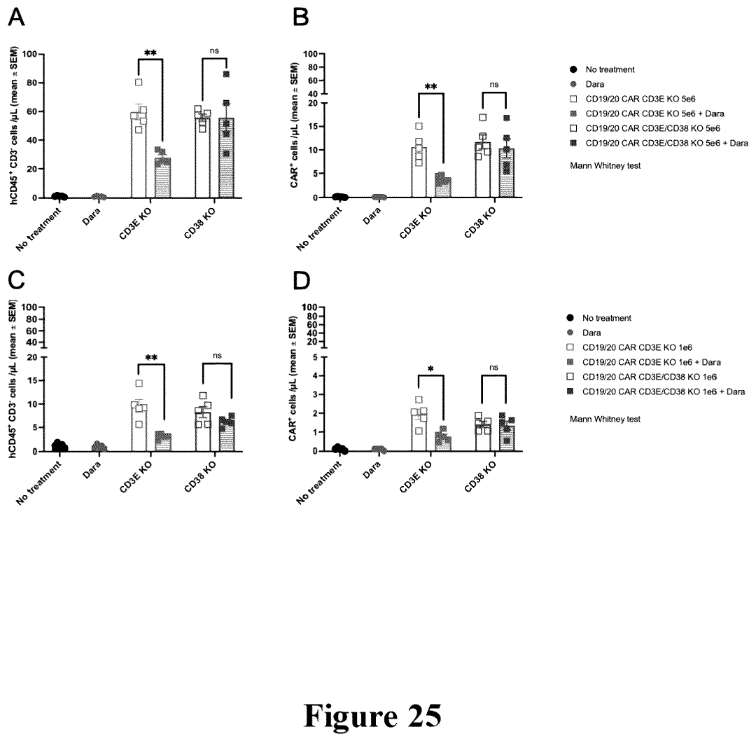

FIG. 25 is experimental data demonstrating CD38 knockout is required to prevent daratumumab-mediated depletion of CAR-T cells. Absolute cells counts in peripheral blood were assessed by flow cytometry at peak expansion, 11 days post CAR-T cell infusion. FIG. 25, panel A shows human CD45+CD3-T cell counts from mice dosed with 5×10⁶ CAR-T cells. FIG. 25, panel B shows CAR+ T cell counts in mice dosed with 5×10⁶ CAR-T cells. FIG. 25, panel C shows Human CD45+CD3-T cells counts from mice dosed with 5×106 CAR-T cells. FIG. 25, panel D CAR+ T cell counts in mice dosed with 1×10⁶ CAR-T cells. Mann Whitney test; ** P<0.001, *P<0.05.

### Example 15: Efficient Knock Out of CD38 with Adenine Base Editing in CAR-T Cells

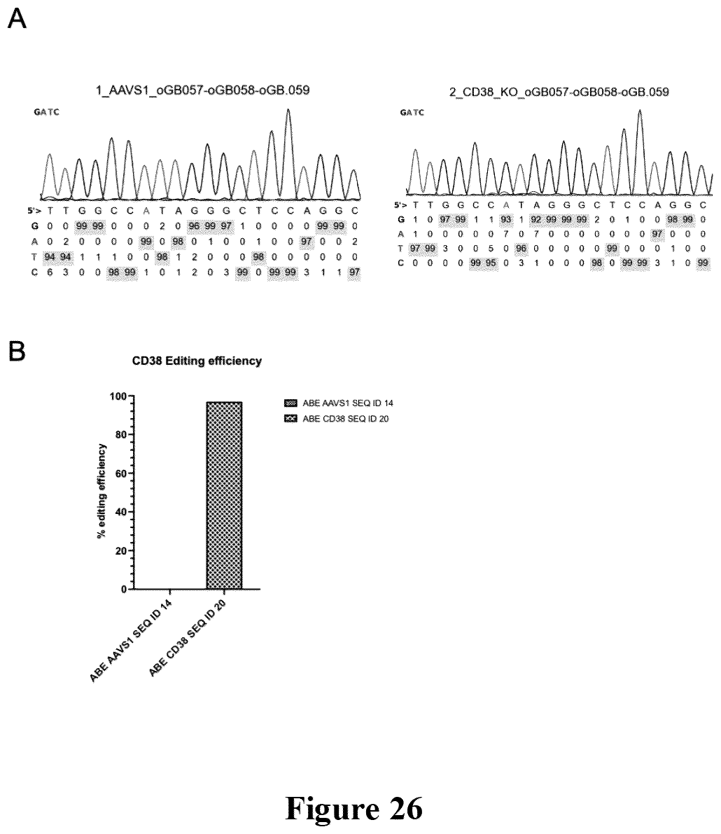

FIG. 26 is experimental data showing efficient knock out of CD38 with adenine base editing with an sgRNA, SEQ ID NO. 20. Interestingly, this sgRNA results in disruption of the CD38 start codon, rather than introducing a stop codon or disrupting canonical mRNA splice sites. FIG. 26, panel A shows analysis of Sanger sequencing to determine base editing efficiency. FIG. 26, panel B shows quantification of A to G editing by sequencing, as shown in FIG. 26, panel A.

### RECITATION OF EMBODIMENTS

Embodiment 1: A cell or a population of cells, comprising an engineered T cell comprising a CD38 expression modification.

Embodiment 2: The cell or population of cells according to embodiment 1, wherein the CD38 expression modification comprises a CD38 gene knock-out.

Embodiment 3: The cell or population of cells according to embodiment 2, wherein the gene knock-out is Cas9 mediated.

Embodiment 4: The cell or population of cells according to embodiment 3, wherein the Cas9 mediated gene knock-out uses a complex comprising a Cas9 molecule and a guide nucleotide sequence.

Embodiment 5: The cell or population of cells according to embodiment 4, wherein the nucleotide guide sequence comprises SEQ ID NO: 1.

Embodiment 6: The cell or population of cells according to embodiment 4, wherein the nucleotide guide sequence comprises of SEQ ID NO: 2.

Embodiment 7: The cell or population of cells according to embodiment 4, wherein the nucleotide guide sequence comprises of SEQ ID NO: 3.

Embodiment 8: The cell or population of cells according to embodiment 4, wherein the nucleotide guide sequence comprises of SEQ ID NO: 4.

Embodiment 9: The cell or population of cells according to embodiment 2, wherein the gene knock-out is Cas 12 mediated.

Embodiment 10: The cell or population of cells according to embodiment 2, wherein the gene knock-out is base editor mediated.

Embodiment 11: The cell or population of cells according to embodiment 10, wherein the base editor comprises a cytosine base editor (CBE).

Embodiment 12: The cell or population of cells according to embodiment 11, wherein the cytosine base editor comprises a cytidine base editor comprised of a Cas9 nickase fused to a cytidine deaminase derived from APOBEC1.

Embodiment 13: The cell or population of cells according to embodiment 10, wherein the base editor comprises an adenine base editor (ABE).

Embodiment 14: The cell or population of cells according to any one of embodiments 10-12, wherein the base editor is complexed with a nucleotide guide sequence.

Embodiment 15: The cell or population of cells of embodiment 14, wherein the nucleotide guide sequence comprises SEQ ID NO: 5.

Embodiment 16: The cell or population of cells of embodiment 14, wherein the nucleotide guide sequence comprises of SEQ ID NO: 6.

Embodiment 17: The cell or population of cells of embodiment 14, wherein the nucleotide guide sequence comprises of SEQ ID NO: 7.

Embodiment 18: The cell or population of cells of embodiment 14, wherein the nucleotide guide sequence comprises of SEQ ID NO: 8.

Embodiment 19: The cell or population of cells of embodiment 14, wherein the nucleotide guide sequence comprises of SEQ ID NO: 9.

Embodiment 20: The cell or population of cells of embodiment 14, wherein the nucleotide guide sequence comprises of SEQ ID NO: 10.

Embodiment 21: The cell or population of cells according to embodiment 1, wherein the CD38 expression modification comprises a CD38 gene knock-in.

Embodiment 22: The cell or population of cells of embodiment 21, wherein the CD38 gene knock-in uses homology directed repair.

Embodiment 23: The cell or population of cells according to any one of the preceding embodiments, wherein the CD38 expression modification uses RNAi.

Embodiment 24: The cell or population of cells according to any one of the preceding embodiments, wherein the CD38 expression modification uses anti-sense oligonucleotide (ASO).

Embodiment 25: The cell or population of cells according to any one of the preceding embodiments, wherein the CD38 expression modification uses epigenetic editing.

Embodiment 26: The cell or population of cells of embodiment 25, wherein the epigenetic editing uses a TALEN.

Embodiment 27: The cell or population of cells of embodiment 25, wherein the epigenetic editing uses a zinc finger nuclease.

Embodiment 28: The cell or population of cells of embodiment 25, wherein the epigenetic editing uses CRISPRi.

Embodiment 29: The cell or population of cells according to any one of the preceding embodiments, wherein the CD38 expression modification comprises a single amino acid substitution.

Embodiment 30: The cell or population of cells of embodiment 29, wherein the single amino acid substitution occurs at an epitope specific for Daratumumab.

Embodiment 31: The cell or population of cells of embodiment 29, wherein the single amino acid substitution occurs at an epitope specific for Isatuximab.

Embodiment 32: The cell or population according to any one of embodiments 29-31, wherein the single amino acid substitution is mediated by a base editor.

Embodiment 33: The cell or population of cells according to any one of embodiments 29-32, wherein the substitution replaces serine 274.

Embodiment 34: The cell or population of cells of embodiment 33, wherein the substitution replaces the serine 274 with a phenylalanine.

Embodiment 35: The cell or population of cells according to any one of embodiments 33 and 34, wherein the base editor comprises a nucleotide guide sequence comprising SEQ ID NO: 11.

Embodiment 36: The cell or population of cells according to any one of embodiments 29-32, wherein the substitution replaces threonine 116.

Embodiment 37: The cell or population of cells of embodiment 36, wherein the substitution replaces the threonine 116 with an alanine.

Embodiment 38: The cell or population of cells according to any one of the preceding embodiments, wherein the base editor comprises a nucleotide guide sequence comprising SEQ ID NO: 16.

Embodiment 39: The cell or population of cells according to any one of the preceding embodiments, wherein the base editor comprises a nucleotide guide sequence comprising SEQ ID NO: 17.

Embodiment 40: The cell or population of cells according to any one of the preceding embodiments, wherein the base editor comprises a nucleotide guide sequence comprising SEQ ID NO: 18.

Embodiment 41: The cell or population of cells according to any one of the preceding embodiments, wherein the base editor comprises a nucleotide guide sequence comprising SEQ ID NO: 19.

Embodiment 42: The cell or population of cells according to any one of the preceding embodiments, wherein the engineered T cell expresses a CD38 antigen nucleotide encoding sequence comprising SEQ ID NO: 21.

Embodiment 43: The cell or population of cells according to any one of the preceding embodiments, wherein the engineered T cell expresses a CD38 antigen protein molecule comprising SEQ ID NO: 23.

Embodiment 44: The cell or population of cells according to any one of the preceding embodiments, wherein the engineered T cell expresses a CD38 antigen protein molecule comprising SEQ ID NO: 24.

Embodiment 45: The cell or population of cells according to any one of the preceding embodiments, wherein the engineered T cell is a CAR T cell.

Embodiment 46: The cell or population of cells of embodiment 45, wherein the CAR T cell is an allogeneic therapeutic.

Embodiment 47: The cell or population of cells of embodiment 45, wherein the CAR T cell is an autologous therapeutic.

Embodiment 48: The cell or population of cells according to any one of the preceding embodiments, wherein the engineered T cell targets CD20.

Embodiment 49: The cell or population of cells according to any one of the preceding embodiments, wherein the engineered T cell targets CD19.

Embodiment 50: The cell or population of cells according to any one of the preceding embodiments, wherein the engineered T cell targets CLL-1.

Embodiment 51: The cell or population of cells according to any one of the preceding embodiments, wherein the engineered T cell targets BCMA.

Embodiment 52: The cell or population of cells according to any one of the preceding embodiments, wherein the engineered T cell targets EGFR.

Embodiment 53: The cell or population of cells according to any one of the preceding embodiments, wherein the engineered T cell targets HER2.

Embodiment 54: The cell or population of cells according to any one of the preceding embodiments, wherein the engineered T cell targets GPC3.

Embodiment 55: The cell or population of cells according to any one of the preceding embodiments, wherein the engineered T cell targets GD2.

Embodiment 56: A system for a combination therapy comprising a T cell and a CD38 targeting compound comprising an engineered T cell comprising a CD38 expression modification and a CD38 targeting compound.

Embodiment 57: The system for a combination therapy comprising a T cell and a CD38 targeting compound of embodiment 56, wherein the CD38 targeting compound comprises an antigen binding molecule having specificity for a CD38 antigen.

Embodiment 58: The system for a combination therapy comprising a T cell and a CD38 targeting compound of embodiment 57, wherein the antigen binding molecule comprises an anti-CD38 antibody.

Embodiment 59: The system for a combination therapy comprising a T cell and a CD38 targeting compound of embodiment 57, wherein the antigen binding molecule comprises a DARPin.

Embodiment 60: The system for a combination therapy comprising a T cell and a CD38 targeting compound of embodiment 57, wherein the antigen binding molecule comprises a nano-body.

Embodiment 61: The system for a combination therapy comprising a T cell and a CD38 targeting compound of embodiment 57, wherein the antigen binding molecule comprises a ligand trap.

Embodiment 62: The system for a combination therapy comprising a T cell and a CD38 targeting compound of embodiment 57, wherein the antigen binding molecule comprises a synthetic component.

Embodiment 63: The system for a combination therapy comprising a T cell and a CD38 targeting compound according to any one of embodiments 56-62, wherein the CD38 targeting compound embodiment isatuximab.

Embodiment 64: The system for a combination therapy comprising a T cell and a CD38 targeting compound according to any one of embodiments 56-62, wherein the CD38 targeting compound comprises daratumumab.

Embodiment 65: The system for a combination therapy comprising a T cell and a CD38 targeting compound according to any one of embodiments 56-62, wherein the CD38 targeting compound comprises MOR202.

Embodiment 66: The system for a combination therapy comprising a T cell and a CD38 compound therapy according to any one of embodiments 56-62, wherein the CD38 targeting compound comprises TAK079.

Embodiment 67: The system for a combination therapy comprising a T cell and a CD38 compound therapy according to any one of embodiments 56-66, wherein the CD38 expression modification comprises a CD38 gene knock-out.

Embodiment 68: The system for a combination therapy comprising a T cell and a CD38 compound therapy according to embodiment 67, wherein the gene knock-out is Cas9 mediated.

Embodiment 69: The system for a combination therapy comprising a T cell and a CD38 compound therapy according to embodiment 68, wherein the Cas9 mediated gene knock-out uses a complex comprising a Cas9 molecule and a guide nucleotide sequence.

Embodiment 70: The system for a combination therapy comprising a T cell and a CD38 compound therapy according to embodiment 69, wherein the nucleotide guide sequence comprises SEQ ID NO: 1.

Embodiment 71: The system for a combination therapy comprising a T cell and a CD38 compound therapy according to embodiment 69, wherein the nucleotide guide sequence comprises of SEQ ID NO: 2.

Embodiment 72: The system for a combination therapy comprising a T cell and a CD38 compound therapy according to embodiment 69, wherein the nucleotide guide sequence comprises SEQ ID NO: 3.

Embodiment 73: The system for a combination therapy comprising a T cell and a CD38 compound therapy according to embodiment 69, wherein the nucleotide guide sequence comprises SEQ ID NO: 4.

Embodiment 74: The system for a combination therapy comprising a T cell and a CD38 compound therapy according to embodiment 67, wherein the gene knock-out is Cas12 mediated.

Embodiment 75: The system for a combination therapy comprising a T cell and a CD38 compound therapy according to embodiment 67, wherein the gene knock-out is base editor mediated.

Embodiment 76: The system for a combination therapy comprising a T cell and a CD38 compound therapy according to embodiment 75, wherein the base editor comprises a cytosine base editor (CBE).

Embodiment 77: The system for a combination therapy comprising a T cell and a CD38 compound therapy according to embodiment 76, wherein the cytosine base editor comprises a cytidine base editor comprised of a Cas9 nickase fused to a cytidine deaminase derived from APOBEC1.

Embodiment 78: The system for a combination therapy comprising a T cell and a CD38 compound therapy according to embodiment 75, wherein the base editor comprises an adenine base editor (ABE).

Embodiment 79: The system for a combination therapy comprising a T cell and a CD38 compound therapy according to any one of embodiments 75-78, wherein the base editor is complexed with a nucleotide guide sequence.

Embodiment 80: The system for a combination therapy comprising a T cell and a CD38 compound therapy according to embodiment 79, wherein the nucleotide guide sequence comprises SEQ ID NO: 5.

Embodiment 81: The system for a combination therapy comprising a T cell and a CD38 compound therapy according to embodiment 79, wherein the nucleotide guide sequence comprises SEQ ID NO: 6.

Embodiment 82: The system for a combination therapy comprising a T cell and a CD38 compound therapy according to embodiment 79, wherein the nucleotide guide sequence comprises SEQ ID NO: 7.

Embodiment 83: The system for a combination therapy comprising a T cell and a CD38 compound therapy according to embodiment 79, wherein the nucleotide guide sequence comprises SEQ ID NO: 8.

Embodiment 84: The system for a combination therapy comprising a T cell and a CD38 compound therapy according to embodiment 79, wherein the nucleotide guide sequence comprises SEQ ID NO: 9.

Embodiment 85: The system for a combination therapy comprising a T cell and a CD38 compound therapy according to embodiment 79, wherein the nucleotide guide sequence comprises SEQ ID NO: 10.

Embodiment 86: The system for a combination therapy comprising a T cell and a CD38 compound therapy according to any one of embodiments 56-85, further comprising a CD38 gene knock-in.

Embodiment 87: The system for a combination therapy comprising a T cell and a CD38 compound therapy according to embodiment 86, wherein the CD38 gene knock-in uses homology directed repair.

Embodiment 88: The system for a combination therapy comprising a T cell and a CD38 compound therapy according to any one of embodiments 56-87, wherein the CD38 expression modification uses RNAi.

Embodiment 89: The system for a combination therapy comprising a T cell and a CD38 compound therapy according to any one of embodiments 56-88, wherein the CD38 expression modification uses anti-sense oligonucleotide (ASO).

Embodiment 90: The system for a combination therapy comprising a T cell and a CD38 compound therapy according to any one of embodiments 56-89, wherein the CD38 expression modification uses epigenetic editing.

Embodiment 91: The system for a combination therapy comprising a T cell and a CD38 compound therapy according to embodiment 90, wherein the epigenetic editing uses a TALEN.

Embodiment 92: The system for a combination therapy comprising a T cell and a CD38 compound therapy according to embodiment 90, wherein the epigenetic editing uses a zinc finger nuclease.

Embodiment 93: The system for a combination therapy comprising a T cell and a CD38 compound therapy according to embodiment 90, wherein the epigenetic editing uses CRISPRi.

Embodiment 94: The system for a combination therapy comprising a T cell and a CD38 compound therapy according to any one of embodiments 56-93, wherein the CD38 expression modification comprises a single amino acid substitution.

Embodiment 95: The system for a combination therapy comprising a T cell and a CD38 compound therapy according to embodiment 94, wherein the single amino acid substitution occurs at an epitope specific for Daratumumab.

Embodiment 96: The system for a combination therapy comprising a T cell and a CD38 compound therapy according to embodiment 94, wherein the single amino acid substitution occurs at an epitope specific for Isatuximab.

Embodiment 97: The system for a combination therapy comprising a T cell and a CD38 compound therapy according to any one of embodiments 94-96, wherein the single amino acid substitution is mediated by a base editor.

Embodiment 98: The system for a combination therapy comprising a T cell and a CD38 compound therapy according to any one of embodiments 94-96, wherein the substitution replaces serine 274.

Embodiment 99: The system for a combination therapy comprising a T cell and a CD38 compound therapy according to embodiment 98, wherein the substitution replaces the serine 274 with a phenylalanine.

Embodiment 100: The system for a combination therapy comprising a T cell and a CD38 compound therapy according to any one of embodiments 98 and 99, wherein the base editor comprises a nucleotide guide sequence comprises SEQ ID NO: 11.

Embodiment 101: The system for a combination therapy comprising a T cell and a CD38 compound therapy according to any one of embodiments 94-96, wherein the substitution replaces threonine 116.

Embodiment 102: The system for a combination therapy comprising a T cell and a CD38 compound therapy according to embodiment 101, wherein the substitution replaces the threonine 116 with an alanine.

Embodiment 103: The system for a combination therapy comprising a T cell and a CD38 compound therapy according to any one of embodiments 56-102, wherein the base editor comprises a nucleotide guide sequence comprises SEQ ID NO: 16.

Embodiment 104: The system for a combination therapy comprising a T cell and a CD38 compound therapy according to any one of embodiments 56-102, wherein the base editor comprises a nucleotide guide sequence comprises SEQ ID NO: 17.

Embodiment 105: The system for a combination therapy comprising a T cell and a CD38 compound therapy according to any one of embodiments 56-102, wherein the base editor comprises a nucleotide guide sequence comprises SEQ ID NO: 18.

Embodiment 106: The system for a combination therapy comprising a T cell and a CD38 compound therapy according to any one of embodiments 56-102, wherein the base editor comprises a nucleotide guide sequence comprises SEQ ID NO: 19.

Embodiment 107: The system for a combination therapy comprising a T cell and a CD38 compound therapy according to any one of embodiments 56-106, wherein the engineered T cell expresses a CD38 antigen nucleotide encoding sequence comprises SEQ ID NO: 21.

Embodiment 108: The system for a combination therapy comprising a T cell and a CD38 compound therapy according to any one of embodiments 56-107, wherein the engineered T cell expresses a CD38 antigen protein molecule comprises SEQ ID NO: 23.

Embodiment 109: The system for a combination therapy comprising a T cell and a CD38 compound therapy according to any one of embodiments 56-108, wherein the engineered T cell expresses a CD38 antigen protein molecule comprises SEQ ID NO: 24.

Embodiment 110: The system for a combination therapy comprising a T cell and a CD38 compound therapy according to any one of embodiments 56-109, wherein the engineered T cell is a CAR T cell.

Embodiment 111: The system for a combination therapy comprising a T cell and a CD38 compound therapy according to any one of embodiments 56-110, wherein the system is an allogeneic therapeutic.

Embodiment 112: The system for a combination therapy comprising a T cell and a CD38 compound therapy according to any one of embodiments 56-111, wherein the system is an autologous therapeutic.

Embodiment 113: The system for a combination therapy comprising a T cell and a CD38 compound therapy according to any one of embodiments 56-112, wherein the engineered T cell targets CD20.

Embodiment 114: The system for a combination therapy comprising a T cell and a CD38 compound therapy according to any one of embodiments 56-113, wherein the engineered T cell targets CD19.

Embodiment 115: The system for a combination therapy comprising a T cell and a CD38 compound therapy according to any one of embodiments 56-114, wherein the engineered T cell targets CLL-1.

Embodiment 116: The system for a combination therapy comprising a T cell and a CD38 compound therapy according to any one of embodiments 56-115, wherein the engineered T cell targets BCMA.

Embodiment 117: The system for a combination therapy comprising a T cell and a CD38 compound therapy according to any one of embodiments 56-116, wherein the engineered T cell targets EGFR.

Embodiment 118: The system for a combination therapy comprising a T cell and a CD38 compound therapy according to any one of embodiments 56-117, wherein the CAR T cells target HER2.

Embodiment 119: The system for a combination therapy comprising a T cell and a CD38 compound therapy according to any one of embodiments 56-118, wherein the engineered T cell targets GPC3.

Embodiment 120: The system for a combination therapy comprising a T cell and a CD38 compound therapy according to any one of embodiments 56-119, wherein the engineered T cell targets GD2.

Embodiment 121: A method of generating a cell therapy treatment, comprising introducing a CD38 expression modification into an engineered T cell.

Embodiment 122: The method of generating a cell therapy treatment of embodiment 121, further comprising activating the engineered T cell.

Embodiment 123: The method of generating a cell therapy treatment according to any one of embodiments 121 and 121, further comprising expanding the engineered T cell.

Embodiment 124: The method of generating a cell therapy treatment according to any one of embodiments 121-123, further comprising transducing the engineered T cell.

Embodiment 125: The method of generating a cell therapy treatment of embodiment 124, wherein the transducing step comprises incubating the T cell with a lentiviral vector.

Embodiment 126: The method of generating a cell therapy treatment according to any one of embodiments 121-125, wherein the CD38 expression modification comprises a CD38 gene knock-out.

Embodiment 127: The method of generating a cell therapy treatment of embodiment 126, wherein the gene knock-out is Cas9 mediated.

Embodiment 128: The method of generating a cell therapy treatment of embodiment 127, wherein the Cas9 mediated gene knock-out uses a complex comprising a Cas9 molecule and a guide nucleotide sequence.

Embodiment 129: The method of generating a cell therapy treatment of embodiment 128, wherein the nucleotide guide sequence comprises SEQ ID NO: 1.

Embodiment 130: The method of generating a cell therapy treatment of embodiment 128, wherein the nucleotide guide sequence comprises SEQ ID NO: 2.

Embodiment 131: The method of generating a cell therapy treatment of embodiment 128, wherein the nucleotide guide sequence comprises SEQ ID NO: 3.

Embodiment 132: The method of generating a cell therapy treatment of embodiment 128, wherein the nucleotide guide sequence comprises SEQ ID NO: 4.

Embodiment 133: The method of generating a cell therapy treatment of embodiment 126, wherein the gene knock-out is Cas12 mediated.

Embodiment 134: The method of generating a cell therapy treatment of embodiment 126, wherein the gene knock-out is base editor mediated.

Embodiment 135: The method of generating a cell therapy treatment of embodiment 134, wherein the base editor comprises a cytosine base editor (CBE).

Embodiment 136: The method of generating a cell therapy treatment of embodiment 135, wherein the cytosine base editor comprises a cytidine base editor comprised of a Cas9 nickase fused to a cytidine deaminase derived from APOBEC1.

Embodiment 137: The method of generating a cell therapy treatment of embodiment 134, wherein the base editor comprises an adenine base editor (ABE).

Embodiment 138: The method of generating a cell therapy treatment according to any one of embodiments 134-137, wherein the base editor is complexed with a nucleotide guide sequence.

Embodiment 139: The method of generating a cell therapy treatment of embodiment 138, wherein the nucleotide guide sequence comprises SEQ ID NO: 5.

Embodiment 140: The method of generating a cell therapy treatment of embodiment 138, wherein the nucleotide guide sequence comprises SEQ ID NO: 6.

Embodiment 141: The method of generating a cell therapy treatment of embodiment 138, wherein the nucleotide guide sequence comprises SEQ ID NO: 7.

Embodiment 142: The method of generating a cell therapy treatment of embodiment 138, wherein the nucleotide guide sequence comprises SEQ ID NO: 8.

Embodiment 143: The method of generating a cell therapy treatment of embodiment 138, wherein the nucleotide guide sequence comprises SEQ ID NO: 9.

Embodiment 144: The method of generating a cell therapy treatment of embodiment 138, wherein the nucleotide guide sequence comprises SEQ ID NO: 10.

Embodiment 145: The method of generating a cell therapy treatment according to any one of embodiments 122-144, further comprising a CD38 gene knock-in.

Embodiment 146: The method of generating a cell therapy treatment of embodiment 145, wherein the CD38 gene knock-in uses homology directed repair.

Embodiment 147: The method of generating a cell therapy treatment according to any one of embodiments 122-148, wherein the CD38 expression modification uses RNAi.

Embodiment 148: The method of generating a cell therapy treatment according to any one of embodiments 122-147, wherein the CD38 expression modification uses anti-sense oligonucleotide (ASO).

Embodiment 149: The method of generating a cell therapy treatment according to any one of embodiments 122-148, wherein the CD38 expression modification uses epigenetic editing.

Embodiment 150: The method of generating a cell therapy treatment of embodiment 145, wherein the epigenetic editing uses a TALEN.

Embodiment 151: The method of generating a cell therapy treatment of embodiment 145, wherein the epigenetic editing uses a zinc finger nuclease.

Embodiment 152: The method of generating a cell therapy treatment of embodiment 145, wherein the epigenetic editing uses CRISPRi.

Embodiment 153: The method of generating a cell therapy treatment according to any one of embodiments 122-153, wherein the CD38 expression modification comprises a single amino acid substitution.

Embodiment 154: The method of generating a cell therapy treatment of embodiment 153, wherein the single amino acid substitution occurs at an epitope specific for Daratumumab.

Embodiment 155: The method of generating a cell therapy treatment of embodiment 153, wherein the single amino acid substitution occurs at an epitope specific for Isatuximab.

Embodiment 156: The method of generating a cell therapy treatment according to any one of embodiments 153-155, wherein the single amino acid substitution is mediated by a base editor.

Embodiment 157: The method of generating a cell therapy treatment according to any one of embodiments 154-156, wherein the substitution replaces serine 274.

Embodiment 158: The method of generating a cell therapy treatment of embodiment 157, wherein the substitution replaces the serine 274 with a phenylalanine.

Embodiment 159: The method of generating a cell therapy treatment according to any one of embodiments 157 and 158, wherein the base editor comprises a nucleotide guide sequence comprising SEQ ID NO: 11.

Embodiment 160: The method of generating a cell therapy treatment according to any one of embodiments 154-156, wherein the substitution replaces threonine 116.

Embodiment 161: The method of generating a cell therapy treatment of embodiment 160, wherein the substitution replaces the threonine 116 with an alanine.

Embodiment 162: The method of generating a cell therapy treatment according to any one of embodiments 121-161, wherein the base editor comprises a nucleotide guide sequence comprises SEQ ID NO: 116.

Embodiment 163: The method of generating a cell therapy treatment according to any one of embodiments 121-161, wherein the base editor comprises a nucleotide guide sequence comprises SEQ ID NO: 17.

Embodiment 164: The method of generating a cell therapy treatment according to any one of embodiments 121-161, wherein the base editor comprises a nucleotide guide sequence comprises SEQ ID NO: 18.

Embodiment 165: The method of generating a cell therapy treatment according to any one of embodiments 121-161, wherein the base editor comprises a nucleotide guide sequence comprises SEQ ID NO: 19.

Embodiment 166: The method of generating a cell therapy treatment according to any one of embodiments 122-165, wherein the engineered T cell expresses a CD38 antigen nucleotide encoding sequence comprises SEQ ID NO: 21.

Embodiment 167: The method of generating a cell therapy treatment according to any one of embodiments 122-166, wherein the engineered T cell expresses a CD38 antigen protein molecule comprises SEQ ID NO: 23.

Embodiment 168: The method of generating a cell therapy treatment according to any one of embodiments 122-167, wherein the engineered T cell expresses a CD38 antigen protein molecule comprises SEQ ID NO: 24.

Embodiment 169: The method of generating a cell therapy treatment according to any one of embodiments 122-168, wherein the engineered T cell is a CAR T cell.

Embodiment 170: The method of generating a cell therapy treatment according to any one of embodiments 122-169, wherein the system is an allogeneic therapeutic.

Embodiment 171: The method of generating a cell therapy treatment according to any one of embodiments 122-170, wherein the system is an autologous therapeutic.

Embodiment 172: The method of generating a cell therapy treatment according to any one of embodiments 122-171, wherein the engineered T cell targets CD20.

Embodiment 173: The method of generating a cell therapy treatment according to any one of embodiments 122-172, wherein the engineered T cell targets CD19.

Embodiment 174: The method of generating a cell therapy treatment according to any one of embodiments 122-173, wherein the engineered T cell targets CLL-1.

Embodiment 175: The method of generating a cell therapy treatment according to any one of embodiments 122-174, wherein the engineered T cell targets BCMA.

Embodiment 176: The method of generating a cell therapy treatment according to any one of embodiments 122-175, wherein the engineered T cell targets EGFR.

Embodiment 177: The method of generating a cell therapy treatment according to any one of embodiments 122-176, wherein the CAR T cells target HER2.

Embodiment 178: The method of generating a cell therapy treatment according to any one of embodiments 122-177, wherein the engineered T cell targets GPC3.

Embodiment 179: The method of generating a cell therapy treatment according to any one of embodiments 122-178, wherein the engineered T cell targets GD2.

Embodiment 180: The method of generating a cell therapy treatment according to any one of embodiments 122-179, wherein the method further comprises combining the CAR T cells with a CD38 targeting compound to generate a cell therapy product.

Embodiment 181: The method of generating a cell therapy treatment of embodiment 180, wherein the CD38 targeting compound comprises an antigen binding molecule having specificity for a CD38 antigen.

Embodiment 182: The method of generating a cell therapy treatment of embodiment 181, wherein the antigen binding molecule comprises an anti-CD38 antibody.

Embodiment 183: The method of generating a cell therapy treatment of embodiment 181, wherein the antigen binding molecule comprises a DARPin.

Embodiment 184: The method of generating a cell therapy treatment of embodiment 181, wherein the antigen binding molecule comprises a nano-body.

Embodiment 185: The method of generating a cell therapy treatment of embodiment 181, wherein the antigen binding molecule comprises a ligand trap.

Embodiment 186: The method of generating a cell therapy treatment of embodiment 181, wherein the antigen binding molecule comprises a synthetic component.

Embodiment 187: The method of generating a cell therapy treatment of embodiment 180, wherein the CD38 targeting compound comprises isatuximab.

Embodiment 188: The method of generating a cell therapy treatment of embodiment 180, wherein the CD38 targeting compound comprises daratumumab.

Embodiment 189: The method of generating a cell therapy treatment of embodiment 180, wherein the CD38 targeting compound comprises MOR202.

Embodiment 190: The method of generating a cell therapy treatment of embodiment 180, wherein the CD38 targeting compound comprises TAK079.

Embodiment 191: A method of treating a patient comprising a T cell and a CD38 targeting compound, comprising administering an engineered T cell comprising a CD38 expression modification to a patient and administering a CD38 targeting compound to the patient.

Embodiment 192: The method of treating a patient using a T cell and a CD38 targeting compound of embodiment 191, wherein the CD38 targeting compound comprises an antigen binding molecule having specificity for a CD38 antigen.

Embodiment 193: The method of treating a patient using a T cell and a CD38 targeting compound of embodiment 192, wherein the antigen binding molecule comprises an anti-CD38 antibody.

Embodiment 194: The method of treating a patient using a T cell and a CD38 targeting compound of embodiment 192, wherein the antigen binding molecule comprises a DARPin.

Embodiment 195: The method of treating a patient using a T cell and a CD38 targeting compound of embodiment 192, wherein the antigen binding molecule comprises a nano-body.

Embodiment 196: The method of treating a patient using a T cell and a CD38 targeting compound of embodiment 192, wherein the antigen binding molecule comprises a ligand trap.

Embodiment 197: The method of treating a patient using a T cell and a CD38 targeting compound of embodiment 192, wherein the antigen binding molecule comprises a synthetic component.

Embodiment 198: The method of treating a patient using a T cell and a CD38 targeting compound according to any one of embodiments 191-197, wherein the CD38 targeting compound comprises Isatuximab.

Embodiment 199: The method of treating a patient using a T cell and a CD38 targeting compound according to any one of embodiments 191-198, wherein the CD38 targeting compound comprises daratumubmab.

Embodiment 200: The method of treating a patient using a T cell and a CD38 targeting compound according to any one of embodiments 191-199, wherein the CD38 targeting compound comprises MOR202.

Embodiment 201: The method of treating a patient using a T cell and a CD38 targeting compound according to any one of embodiments 191-200, wherein the CD38 targeting compound comprises TAK079.

Embodiment 202: The method of treating a patient using a T cell and a CD38 targeting compound according to any one of embodiments 191-201, wherein the CD38 expression modification comprises a CD38 gene knock-out.

Embodiment 203: The method of treating a patient using a T cell and a CD38 targeting compound according to embodiment 202, wherein the gene knock-out is Cas9 mediated.

Embodiment 204: The method of treating a patient using a T cell and a CD38 targeting compound according to embodiment 203, wherein the Cas9 mediated gene knock-out uses a complex comprising a Cas9 molecule and a guide nucleotide sequence.

Embodiment 205: The method of treating a patient using a T cell and a CD38 targeting compound according to embodiment 204, wherein the nucleotide guide sequence comprises SEQ ID NO: 1.

Embodiment 206: The method of treating a patient using a T cell and a CD38 targeting compound according to embodiment 204, wherein the nucleotide guide sequence comprises SEQ ID NO: 2.

Embodiment 207: The method of treating a patient using a T cell and a CD38 targeting compound according to embodiment 204, wherein the nucleotide guide sequence comprises SEQ ID NO: 3.

Embodiment 208: The method of treating a patient using a T cell and a CD38 targeting compound according to embodiment 204, wherein the nucleotide guide sequence comprises SEQ ID NO: 4.

Embodiment 209: The method of treating a patient using a T cell and a CD38 targeting compound according to embodiment 202, wherein the gene knock-out is Cas12 mediated.

Embodiment 210: The method of treating a patient using a T cell and a CD38 targeting compound according to embodiment 202, wherein the gene knock-out is base editor mediated.

Embodiment 211: The method of treating a patient using a T cell and a CD38 targeting compound according to embodiment 210, wherein the base editor comprises a cytosine base editor (CBE).

Embodiment 212: The method of treating a patient using a T cell and a CD38 targeting compound according to embodiment 210, wherein the cytosine base editor comprises a cytidine base editor comprised of a Cas9 nickase fused to a cytidine deaminase derived from APOBEC1.

Embodiment 213: The method of treating a patient using a T cell and a CD38 targeting compound according to embodiment 210, wherein the base editor comprises an adenine base editor (ABE).

Embodiment 214: The method of treating a patient using a T cell and a CD38 targeting compound according to any one of embodiments 210⁻²¹³, wherein the base editor is complexed with a nucleotide guide sequence.

Embodiment 215: The method of treating a patient using a T cell and a CD38 targeting compound according to embodiment 214, wherein the nucleotide guide sequence comprises SEQ ID NO: 5.

Embodiment 216: The method of treating a patient using a T cell and a CD38 targeting compound according to embodiment 214, wherein the nucleotide guide sequence comprises SEQ ID NO: 6.

Embodiment 217: The method of treating a patient using a T cell and a CD38 targeting compound according to embodiment 214, wherein the nucleotide guide sequence comprises SEQ ID NO: 7.

Embodiment 218: The method of treating a patient using a T cell and a CD38 targeting compound according to embodiment 214, wherein the nucleotide guide sequence comprises SEQ ID NO: 8.

Embodiment 219: The method of treating a patient using a T cell and a CD38 targeting compound according to embodiment 214, wherein the nucleotide guide sequence comprises SEQ ID NO: 9.

Embodiment 220: The method of treating a patient using a T cell and a CD38 targeting compound according to embodiment 214, wherein the nucleotide guide sequence comprises SEQ ID NO: 10.

Embodiment 221: The method of treating a patient using a T cell and a CD38 targeting compound according to any one of embodiments 191-220, further comprising a CD38 gene knock-in.

Embodiment 222: The method of treating a patient using a T cell and a CD38 targeting compound according to embodiment 221, wherein the CD38 gene knock-in uses homology directed repair.

Embodiment 223: The method of treating a patient using a T cell and a CD38 targeting compound according to any one of embodiments 191-222, wherein the CD38 expression modification uses RNAi.

Embodiment 224: The method of treating a patient using a T cell and a CD38 targeting compound according to any one of embodiments 191-223, wherein the CD38 expression modification uses anti-sense oligonucleotide (ASO).

Embodiment 225: The method of treating a patient using a T cell and a CD38 targeting compound according to any one of embodiments 191-224, wherein the CD38 expression modification uses epigenetic editing.

Embodiment 226: The method of treating a patient using a T cell and a CD38 targeting compound according to embodiment 225, wherein the epigenetic editing uses a TALEN.

Embodiment 227: The method of treating a patient using a T cell and a CD38 targeting compound according to embodiment 225, wherein the epigenetic editing uses a zinc finger nuclease.

Embodiment 228: The method of treating a patient using a T cell and a CD38 targeting compound according to embodiment 225, wherein the epigenetic editing uses CRISPRi.

Embodiment 229: The method of treating a patient using a T cell and a CD38 targeting compound according to any one of embodiments 191-228, wherein the CD38 expression modification comprises a single amino acid substitution.

Embodiment 230: The method of treating a patient using a T cell and a CD38 targeting compound according to embodiment 229, wherein the single amino acid substitution occurs at an epitope specific for Daratumumab.

Embodiment 231: The method of treating a patient using a T cell and a CD38 targeting compound according to embodiment 229, wherein the single amino acid substitution occurs at an epitope specific for Isatuximab.

Embodiment 232: The method of treating a patient using a T cell and a CD38 targeting compound according to any one of embodiments 229-231, wherein the single amino acid substitution is mediated by a base editor.

Embodiment 233: The method of treating a patient using a T cell and a CD38 targeting compound according to any one of embodiments 229-232, wherein the substitution replaces serine 274.

Embodiment 234: The method of treating a patient using a T cell and a CD38 targeting compound according to embodiment 233, wherein the substitution replaces the serine 274 with a phenylalanine.

Embodiment 235: The method of treating a patient using a T cell and a CD38 targeting compound according to any one of embodiments 232-234, wherein the base editor comprises a nucleotide guide sequence comprising SEQ ID NO: 11.

Embodiment 236: The method of treating a patient using a T cell and a CD38 targeting compound according to any one of embodiments 229-232, wherein the substitution replaces threonine 116.

Embodiment 237: The method of treating a patient using a T cell and a CD38 targeting compound according to embodiment 236, wherein the substitution replaces the threonine 116 with an alanine.

Embodiment 238: The method of treating a patient using a T cell and a CD38 targeting compound according to any one of embodiments 236 and 337, wherein the base editor comprises a nucleotide guide sequence comprising SEQ ID NO: 16.

Embodiment 239: The method of treating a patient using a T cell and a CD38 targeting compound according to any one of embodiments 236 and 337, wherein the base editor comprises a nucleotide guide sequence comprising SEQ ID NO: 17.

Embodiment 240: The method of treating a patient using a T cell and a CD38 targeting compound according to any one of embodiments 236 and 337, wherein the base editor comprises a nucleotide guide sequence comprising SEQ ID NO: 18.

Embodiment 241: The method of treating a patient using a T cell and a CD38 targeting compound according to any one of embodiments 236 and 337, wherein the base editor comprises a nucleotide guide sequence comprising SEQ ID NO: 19.

Embodiment 242: The method of treating a patient using a T cell and a CD38 targeting compound according to any one of embodiments 191-241, wherein the engineered T cell expresses a CD38 antigen nucleotide encoding a sequence comprising SEQ ID NO: 21.

Embodiment 243: The method of treating a patient using a T cell and a CD38 targeting compound according to any one of embodiments 191-241, wherein the engineered T cell expresses a CD38 antigen protein molecule comprising SEQ ID NO: 23.

Embodiment 244: The method of treating a patient using a T cell and a CD38 targeting compound according to any one of embodiments 191-241, wherein the engineered T cell expresses a CD38 antigen protein molecule comprising SEQ ID NO: 24.

Embodiment 245: The method of treating a patient using a T cell and a CD38 targeting compound according to any one of embodiments 191-244, wherein the engineered T cell is a CAR T cell.

Embodiment 246: The method of treating a patient using a T cell and a CD38 targeting compound according to any one of embodiments 191-245, wherein the method is an allogeneic therapeutic.

Embodiment 247: The method of treating a patient using a T cell and a CD38 targeting compound according to any one of embodiments 191-246, wherein the method is an autologous therapeutic.

Embodiment 248: The method of treating a patient using a T cell and a CD38 targeting compound according to any one of embodiments 191-247, wherein the engineered T cell targets CD20.

Embodiment 249: The method of treating a patient using a T cell and a CD38 targeting compound according to any one of embodiments 191-248, wherein the engineered T cell targets CD19.

Embodiment 250: The method of treating a patient using a T cell and a CD38 targeting compound according to any one of embodiments 191-249, wherein the engineered T cell targets CLL-1.

Embodiment 251: The method of treating a patient using a T cell and a CD38 targeting compound according to any one of embodiments 191-250, wherein the engineered T cell targets BCMA.

Embodiment 252: The method of treating a patient using a T cell and a CD38 targeting compound according to any one of embodiments 191-251, wherein the engineered T cell targets EGFR.

Embodiment 253: The method of treating a patient using a T cell and a CD38 targeting compound according to any one of embodiments 191-252, wherein the CAR T cells target HER2.

Embodiment 254: The method of treating a patient using a T cell and a CD38 targeting compound according to any one of embodiments 191-253, wherein the engineered T cell targets GPC3.

Embodiment 255: The method of treating a patient using a T cell and a CD38 targeting compound according to any one of embodiments 191-254, wherein the engineered T cell targets GD2.

Embodiment 256: A kit for a combination therapy using a T cell and a CD38 targeting compound, comprising an engineered T cell comprising a CD38 expression modification.

Embodiment 257: The kit for a combination therapy using a T cell and a CD38 targeting compound of embodiment 256, further comprising a CD38 targeting compound.

Embodiment 258: The kit for a combination therapy using a T cell and a CD38 targeting compound of embodiment 257, wherein the CD38 targeting compound comprises an antigen binding molecule having specificity for a CD38 antigen.

Embodiment 259: The kit for a combination therapy using a T cell and a CD38 targeting compound of embodiment 258, wherein the antigen binding molecule comprises an anti-CD38 antibody.

Embodiment 260: The kit for a combination therapy using a T cell and a CD38 targeting compound of embodiment 259, wherein the antigen binding molecule comprises a DARPin.

Embodiment 261: The kit for a combination therapy using a T cell and a CD38 targeting compound of embodiment 260, wherein the antigen binding molecule comprises a nano-body.

Embodiment 262: The kit for a combination therapy using a T cell and a CD38 targeting compound of embodiment 261, wherein the antigen binding molecule comprises a ligand trap.

Embodiment 263: The kit for a combination therapy using a T cell and a CD38 targeting compound of embodiment 264, wherein the antigen binding molecule comprises a synthetic component.

Embodiment 264: The kit for a combination compound using a T cell and a CD38 targeting compound according to any one of embodiments 256-263, wherein the CD38 targeting compound comprises Isatuximab.

Embodiment 265: The kit for a combination compound using a T cell and a CD38 targeting compound according to any one of embodiments 256-263, wherein the CD38 targeting compound comprises daratumubmab.

Embodiment 266: The kit for a combination compound using a T cell and a CD38 targeting compound according to any one of embodiments 256-263, wherein the CD38 targeting compound comprises MOR202.

Embodiment 267: The kit for a combination compound using a T cell and a CD38 targeting compound according to any one of embodiments 256-263, wherein the CD38 targeting compound comprises TAK079.

Embodiment 268: The kit for a combination compound using a T cell and a CD38 targeting compound according to any one of embodiments 256-267, wherein the CD38 expression modification comprises a CD38 gene knock-out.

Embodiment 269: The kit for a combination compound using a T cell and a CD38 targeting compound according to embodiment 268, wherein the gene knock-out is Cas9 mediated.

Embodiment 270: The kit for a combination compound using a T cell and a CD38 targeting compound according to embodiment 269, wherein the Cas9 mediated gene knock-out uses a complex comprising a Cas9 molecule and a guide nucleotide sequence.

Embodiment 271: The kit for a combination compound using a T cell and a CD38 targeting compound according to embodiment 270, wherein the nucleotide guide sequence comprises SEQ ID NO: 1.

Embodiment 272: The kit for a combination compound using a T cell and a CD38 targeting compound according to embodiment 270, wherein the nucleotide guide sequence comprises SEQ ID NO: 2.

Embodiment 273: The kit for a combination compound using a T cell and a CD38 targeting compound according to embodiment 270, wherein the nucleotide guide sequence comprises SEQ ID NO: 3.

Embodiment 274: The kit for a combination compound using a T cell and a CD38 targeting compound according to embodiment 270, wherein the nucleotide guide sequence comprises SEQ ID NO: 4.

Embodiment 275: The kit for a combination compound using a T cell and a CD38 targeting compound according to embodiment 268, wherein the gene knock-out is Cas12 mediated.

Embodiment 276: The kit for a combination compound using a T cell and a CD38 targeting compound according to embodiment 268, wherein the gene knock-out is base editor mediated.

Embodiment 277: The kit for a combination compound using a T cell and a CD38 targeting compound according to embodiment 276, wherein the base editor comprises a cytosine base editor (CBE).

Embodiment 278: The kit for a combination compound using a T cell and a CD38 targeting compound according to embodiment 277, wherein the cytosine base editor comprises a cytidine base editor comprised of a Cas9 nickase fused to a cytidine deaminase derived from APOBEC1.

Embodiment 279: The kit for a combination compound using a T cell and a CD38 targeting compound according to embodiment 276, wherein the base editor comprises an adenine base editor (ABE).

Embodiment 280: The kit for a combination compound using a T cell and a CD38 targeting compound according to any one of embodiments 276-279, wherein the base editor is complexed with a nucleotide guide sequence.

Embodiment 281: The kit for a combination compound using a T cell and a CD38 targeting compound according to embodiment 280, wherein the nucleotide guide sequence comprises SEQ ID NO: 5.

Embodiment 282: The system for a combination therapy using a T cell and a CD38 compound therapy according to embodiment 280, wherein the nucleotide guide sequence comprises SEQ ID NO: 6.

Embodiment 283: The kit for a combination compound using a T cell and a CD38 targeting compound according to embodiment 280, wherein the nucleotide guide sequence comprises SEQ ID NO: 7.

Embodiment 284: The kit for a combination compound using a T cell and a CD38 targeting compound according to embodiment 280, wherein the nucleotide guide sequence comprises SEQ ID NO: 8.

Embodiment 285: The kit for a combination compound using a T cell and a CD38 targeting compound according to embodiment 280, wherein the nucleotide guide sequence comprises SEQ ID NO: 9.

Embodiment 286: The kit for a combination compound using a T cell and a CD38 targeting compound according to embodiment 280, wherein the nucleotide guide sequence comprises SEQ ID NO: 10.

Embodiment 287: The kit for a combination compound using a T cell and a CD38 targeting compound according to any one of embodiments 256-286 a CD38 gene knock-in.

Embodiment 288: The kit for a combination compound using a T cell and a CD38 targeting compound according to embodiment 287, wherein the CD38 gene knock-in uses homology directed repair.

Embodiment 289: The kit for a combination compound using a T cell and a CD38 targeting compound according to any one of embodiments 256-288, wherein the CD38 expression modification uses RNAi.

Embodiment 290: The kit for a combination compound using a T cell and a CD38 targeting compound according to any one of embodiments 256-289, wherein the CD38 expression modification uses anti-sense oligonucleotide (ASO).

Embodiment 291: The kit for a combination compound using a T cell and a CD38 targeting compound according to any one of embodiments 256-290, wherein the CD38 expression modification uses epigenetic editing.

Embodiment 292: The kit for a combination compound using a T cell and a CD38 targeting compound according to embodiment 291, wherein the epigenetic editing uses a TALEN.

Embodiment 293: The kit for a combination compound using a T cell and a CD38 targeting compound according to embodiment 291, wherein the epigenetic editing uses a zinc finger nuclease.

Embodiment 294: The kit for a combination compound using a T cell and a CD38 targeting compound according to embodiment 291, wherein the epigenetic editing uses CRISPRi.

Embodiment 295: The kit for a combination compound using a T cell and a CD38 targeting compound according to any one of embodiments 256-294, wherein the CD38 expression modification comprises a single amino acid substitution.

Embodiment 296: The kit for a combination compound using a T cell and a CD38 targeting compound according to embodiment 295, wherein the single amino acid substitution occurs at an epitope specific for Daratumumab.

Embodiment 297: The kit for a combination compound using a T cell and a CD38 targeting compound according to embodiment 295, wherein the single amino acid substitution occurs at an epitope specific for Isatuximab.

Embodiment 298: The kit for a combination compound using a T cell and a CD38 targeting compound according to any one of embodiments 295-297, wherein the single amino acid substitution is mediated by a base editor.

Embodiment 299: The kit for a combination compound using a T cell and a CD38 targeting compound according to any one of embodiments 295-297, wherein the substitution replaces serine 274.

Embodiment 300: The kit for a combination compound using a T cell and a CD38 targeting compound according to embodiment 299, wherein the substitution replaces the serine 274 with a phenylalanine.

Embodiment 301: The kit for a combination compound using a T cell and a CD38 targeting compound according to any one of embodiments 299 and 300, wherein the base editor comprises a nucleotide guide sequence comprising SEQ ID NO: 11.

Embodiment 302: The kit for a combination compound using a T cell and a CD38 targeting compound according to any one of embodiments 295-297, wherein the substitution replaces threonine 116.

Embodiment 303: The kit for a combination compound using a T cell and a CD38 targeting compound according to embodiment 302, wherein the substitution replaces the threonine 116 with an alanine.

Embodiment 304: The kit for a combination compound using a T cell and a CD38 targeting compound according to any one of embodiments 256-303, wherein the base editor comprises a nucleotide guide sequence comprising SEQ ID NO: 16.

Embodiment 305: The kit for a combination compound using a T cell and a CD38 targeting compound according to any one of embodiments 256-303, wherein the base editor comprises a nucleotide guide sequence comprising of SEQ ID NO: 17.

Embodiment 306: The kit for a combination compound using a T cell and a CD38 targeting compound according to any one of embodiments 256-303, wherein the base editor comprises a nucleotide guide sequence comprising SEQ ID NO: 18.

Embodiment 307: The kit for a combination compound using a T cell and a CD38 targeting compound according to any one of embodiments 256-303, wherein the base editor comprises a nucleotide guide sequence comprising of SEQ ID NO: 19.

Embodiment 308: The kit for a combination compound using a T cell and a CD38 targeting compound according to any one of embodiments 256-307, wherein the engineered T cell expresses a CD38 antigen nucleotide encoding sequence comprising SEQ ID NO: 21.

Embodiment 309: The kit for a combination compound using a T cell and a CD38 targeting compound according to any one of embodiments 256-307, wherein the engineered T cell expresses a CD38 antigen protein molecule comprising SEQ ID NO: 23.

Embodiment 310: The kit for a combination compound using a T cell and a CD38 targeting compound according to any one of embodiments 256-307, wherein the engineered T cell expresses a CD38 antigen protein molecule comprising SEQ ID NO: 24.

Embodiment 311: The kit for a combination compound using a T cell and a CD38 targeting compound according to any one of embodiments 256-310, wherein the engineered T cell is a CAR T cell.

Embodiment 312: The kit for a combination compound using a T cell and a CD38 targeting compound according to any one of embodiments 256-311, wherein the kit comprises an allogeneic therapeutic.

Embodiment 313: The kit for a combination compound using a T cell and a CD38 targeting compound according to any one of embodiments 256-312, wherein the kit comprises an autologous therapeutic.

Embodiment 314: The kit for a combination compound using a T cell and a CD38 targeting compound according to any one of embodiments 256-313, wherein the engineered T cell targets CD20.

Embodiment 315: The kit for a combination compound using a T cell and a CD38 targeting compound according to any one of embodiments 256-314, wherein the engineered T cell targets CD19.

Embodiment 316: The kit for a combination compound using a T cell and a CD38 targeting compound according to any one of embodiments 256-315, wherein the engineered T cell targets CLL-1.

Embodiment 317: The kit for a combination therapy using a T cell and a CD38 compound therapy according to any one of embodiments 256-316, wherein the engineered T cell targets BCMA.

Embodiment 318: The kit for a combination therapy using a T cell and a CD38 compound therapy according to any one of embodiments 256-317, wherein the engineered T cell targets EGFR.

Embodiment 319: The kit for a combination therapy using a T cell and a CD38 compound therapy according to any one of embodiments 256-318, wherein the engineered T cell targets HER2.

Embodiment 320: The kit for a combination therapy using a T cell and a CD38 compound therapy according to any one of embodiments 256-319, wherein the engineered T cell targets GPC3.

Embodiment 321: The kit for a combination therapy using a T cell and a CD38 compound therapy according to any one of embodiments 256-320, wherein the engineered T cell targets GD2.

### INCORPORATION BY REFERENCE

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention. To the extent that any of the definitions or terms provided in the references incorporated by reference differ from the terms and discussion provided herein, the present terms and definitions control.

The foregoing written specification is considered to be sufficient to enable one skilled in the art to practice the invention. The foregoing description and Examples that follow detail certain preferred embodiments of the invention and describe the best mode contemplated by the inventors. It will be appreciated, however, that no matter how detailed the foregoing may appear in text, the invention may be practiced in many ways and the invention should be construed in accordance with the appended claims and any equivalents thereof.

1. A cell or a population of cells, comprising:
an engineered T cell comprising a CD38 expression modification.

2. The cell or population of cells of claim 1, wherein the CD38 expression modification comprises:
a CD38 gene knock-out.

3. The cell or population of cells of claim 2, wherein the CD38 expression modification comprises a single amino acid substitution.

4. The cell or population of cells of claim 3, wherein the single amino acid substitution occurs at an epitope specific for Daratumumab.

5. The cell or population of cells of claim 3, wherein the single amino acid substitution occurs at an epitope specific for Isatuximab.

6. The cell or population of cells of claim 3, wherein the single amino acid substitution is mediated by a base editor.

7. The cell or population of cells of claim 3, wherein the substitution replaces serine 274 with a phenylalanine.

8. The cell or population of cells of claim 3, wherein the substitution replaces a threonine 116 with an alanine.

9. The cell or population of cells of claim 1, wherein the engineered T cell comprises a CAR T cell.

10. The cell or population of cells of claim 1, wherein the engineered T cell is an allogeneic therapeutic.

11. The cell or population of cells of claim 1, wherein the engineered T cell is modified to target at least one of CD19, CD20, CLL-1, BCMA, EGFR, HER2, GPC3, or GD2.

12. A system for a combination therapy comprising a T cell and a CD38 targeting compound, comprising:
an engineered T cell comprising a CD38 expression modification; and
a CD38 targeting compound.

13. The system for a combination therapy comprising a T cell and a CD38 targeting compound of claim 12, wherein the CD38 targeting compound comprises an anti-CD38 antibody.

14. The system for a combination therapy comprising a T cell and a CD38 targeting compound of claim 13, wherein the CD38 targeting compound comprises at least one of a DARPin, a nano-body, a ligand trap, or a synthetic component.

15. The system for a combination therapy comprising a T cell and a CD38 targeting compound of claim 12, wherein the CD38 targeting compound comprises isatuximab.

16. The system for a combination therapy comprising a T cell and a CD38 targeting compound of claim 12, wherein the CD38 targeting compound comprises daratumumab.

17. The system for a combination therapy comprising a T cell and a CD38 compound of claim 12, wherein the CD38 expression modification comprises:
a CD38 gene knock-out.

18. The system for a combination therapy comprising a T cell and a CD38 compound of claim 12, wherein the engineered T cell is modified to target at least one of CD19, CD20, CLL-1, BCMA, EGFR, HER2, GPC3, or GD2.

19. A method of generating a cell therapy treatment, comprising:
introducing a CD38 expression modification into an engineered T cell.

20. The method of generating a cell therapy treatment of claim 1918, wherein the step of introducing further comprises knocking out a CD38 gene of the engineered T cell.

21. The method of generating a cell therapy treatment of claim 20, wherein the step of knocking out the CD38 gene further comprises applying a base editor to the engineered T cell.

22. (canceled)

23. The method of generating a cell therapy treatment of claim 12, wherein the engineered T cell is a CAR T cell.

24. The method of generating a cell therapy treatment of claim 12, further comprising engineering the engineered T cell to target at least one of CD19, CD20, CLL-1, BCMA, EGFR, HER2, GPC3, or GD2.

25. The method of generating a cell therapy treatment of claim 12, comprising:
administering the engineered T cell to a patient; and
administering a CD38 targeting compound to the patient.

26. The method of generating a cell therapy treatment of claim 25, wherein the CD38 targeting compound comprises an anti-CD38 antibody.

27. (canceled)
